SEQUENCE LISTING (65 sequences)

SEQ ID NO: 1 - protein, 140 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAACHFCDSLPKTQDEYILWGQGTQVTVSSAAAYPYDVPDYGS

SEQ ID NO: 2 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 3 - protein, 137 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRDYSLSSSSDDFDYWGQGTQVTVSSAAAYPYDVPDYGS

SEQ ID NO: 4 - protein, 140 aa, synthetic construct
  VARIANT 102: note = Fluorosulfonyltyrosine (FSY), fluorosulfonyloxybenzoyl-L-lysine (FSK), fluoro-fluorosulfonyltyrosine (FFY), or any derivative thereof
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAXFADSLPKTQDEYILWGQGTQVTVSSAAAYPYDVPDYGS

SEQ ID NO: 5 - protein, 10 aa, synthetic construct
GYTDSNYYMS

SEQ ID NO: 6 - protein, 16 aa, synthetic construct
VNTGRGSTSYADSVKG

SEQ ID NO: 7 - protein, 18 aa, synthetic construct
AACHFCDSLPKTQDEYIL

SEQ ID NO: 8 - protein, 10 aa, synthetic construct
RFMISEYSMH

SEQ ID NO: 9 - protein, 16 aa, synthetic construct
TINPAGTTDYAESVKG

SEQ ID NO: 10 - protein, 5 aa, synthetic construct
DGYGY

SEQ ID NO: 11 - protein, 10 aa, synthetic construct
GWPYSTYSMN

SEQ ID NO: 12 - protein, 17 aa, synthetic construct
GISSTMSGIIFAESKAG

SEQ ID NO: 13 - protein, 15 aa, synthetic construct
RRDYSLSSSSDDFDY

SEQ ID NO: 14 - protein, 10 aa, synthetic construct
GGGGSGGGGS

SEQ ID NO: 15 - protein, 24 aa, synthetic construct
MKYLLPTAAAGLLLLAAQPAMAMG

SEQ ID NO: 16 - protein, 126 aa, synthetic construct
MGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 17 - protein, 124 aa, synthetic construct
  VARIANT 109: note = Fluorosulfonyltyrosine (FSY), fluorosulfonyloxybenzoyl-L-lysine (FSK), fluoro-fluorosulfonyltyrosine (FFY), or any derivative thereof
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSS

SEQ ID NO: 18 - protein, 130 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 19 - protein, 264 aa, synthetic construct
  VARIANT 109: note = Fluorosulfonyltyrosine (FSY), fluorosulfonyloxybenzoyl-L-lysine (FSK), fluoro-fluorosulfonyltyrosine (FFY), or any derivative thereof
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 20 - protein, 140 aa, synthetic construct
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGS

SEQ ID NO: 21 - protein, 290 aa, synthetic construct
  VARIANT 102: note = Fluorosulfonyltyrosine (FSY), fluorosulfonyloxybenzoyl-L-lysine (FSK), fluoro-fluorosulfonyltyrosine (FFY), or any derivative thereof
QVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAXFADSLPKTQDEYILWGQGTQVTVSSAAAYPYDVPDYGSGGGGSGGGGSQVQLQESGGGSVQAGGSLRLSCTAPGYTDSNYYMSWFRQAPGKEREWVAGVNTGRGSTSYADSVKGRFTISQDNAKNTMFLQMNSLKPEDTAIYYCAVAAAHFADSLPKTQDEYILWGQGTQVTVSSAAAYPYDVPDYGS

SEQ ID NO: 22 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 23 - protein, 260 aa, synthetic construct
MGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 24 - protein, 260 aa, synthetic construct
  VARIANT 111: note = Fluorosulfonyltyrosine (FSY), fluorosulfonyloxybenzoyl-L-lysine (FSK), fluoro-fluorosulfonyltyrosine (FFY), or any derivative thereof
MGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 25 - protein, 119 aa, synthetic construct
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSS

SEQ ID NO: 26 - protein, 260 aa, synthetic construct
MGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSEVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSS

SEQ ID NO: 27 - protein, 260 aa, synthetic construct
  VARIANT 111: note = Fluorosulfonyltyrosine (FSY), fluorosulfonyloxybenzoyl-L-lysine (FSK), fluoro-fluorosulfonyltyrosine (FFY), or any derivative thereof
MGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGGGSGGGGSGGGGSEVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSS

SEQ ID NO: 28 - protein, 245 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 29 - protein, 111 aa, synthetic construct
  VARIANT 54: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGGSLTLSCAASRFMISEYSMHWVRQAPGKGLEWVSTINPXGTTDYAESVKGRFTISRDNAKNTLYLQMNSLKPEDTAVYYCDGYGYRGQGTQVTVSS

SEQ ID NO: 30 - protein, 245 aa, synthetic construct
  VARIANT 54: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGGSLTLSCAASRFMISEYSMHWVRQAPGKGLEWVSTINPXGTTDYAESVKGRFTISRDNAKNTLYLQMNSLKPEDTAVYYCDGYGYRGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 31 - protein, 15 aa, synthetic construct
GGGGSGGGGSGGGGS

SEQ ID NO: 32 - protein, 20 aa, synthetic construct
GGGGSGGGGSGGGGSGGGGS

SEQ ID NO: 33 - protein, 25 aa, synthetic construct
GGGGSGGGGSGGGGSGGGGSGGGGS

SEQ ID NO: 34 - protein, 14 aa, synthetic construct
SPSTPPTPSPSTPP

SEQ ID NO: 35 - protein, 291 aa, synthetic construct
  VARIANT 235: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 36 - protein, 286 aa, synthetic construct
  VARIANT 230: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 37 - protein, 296 aa, synthetic construct
  VARIANT 240: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 38 - protein, 301 aa, synthetic construct
  VARIANT 245: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSGGGGSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 39 - protein, 290 aa, synthetic construct
  VARIANT 234: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSSPSTPPTPSPSTPPQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 40 - protein, 296 aa, synthetic construct
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPHHHHHH

SEQ ID NO: 41 - protein, 301 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPHHHHHH

SEQ ID NO: 42 - protein, 301 aa, synthetic construct
  VARIANT 245: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 43 - protein, 297 aa, synthetic construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPHHHHHH

SEQ ID NO: 44 - protein, 294 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPHHHHHH

SEQ ID NO: 45 - protein, 294 aa, synthetic construct
  VARIANT 238: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 46 - protein, 301 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSGGGGSGGGGSGGGGSGGGGSEVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTLVTVSSAAAYPYDVPDYGSSPSTPPTPSPSTPPHHHHHH

SEQ ID NO: 47 - protein, 297 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSGGGGSGGGGSGGGGSGGGGSEVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSAAAYPYDVPDYGSSPSTPPTPSPSTPPHHHHHH

SEQ ID NO: 48 - protein, 294 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSGGGGSGGGGSGGGGSGGGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSAAAYPYDVPDYGSSPSTPPTPSPSTPPHHHHHH

SEQ ID NO: 49 - protein, 294 aa, synthetic construct
  VARIANT 101: note = Fluorosulfonyltyrosine (FSY), fluorosulfonyloxybenzoyl-L-lysine (FSK), fluoro-fluorosulfonyltyrosine (FFY), or any derivative thereof
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSGGGGSGGGGSGGGGSGGGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSAAAYPYDVPDYGSSPSTPPTPSPSTPPHHHHHH

SEQ ID NO: 50 - protein, 136 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRFSLSSSSDDFDYWGQGTQVTVSSAAAYPYDVPDYGS

SEQ ID NO: 51 - protein, 157 aa, synthetic construct
QVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRYFSLSSSSDDFDYWGQGTQVTVSSAAAYPYDVPDYGSSPSTPPTPSPSTPPHHHHHH

SEQ ID NO: 52 - protein, 119 aa, synthetic construct; identical to SEQ ID NO: 25

SEQ ID NO: 53 - protein, 124 aa, synthetic construct
EVQLVESGGGPVQAGGSLRLSCAASGRTYRGYSMGWFRQAPGKEREFVAAIVWSGGNTYYEDSVKGRFTISRDNAKNTMYLQMTSLKPEDSATYYCAAKIRPYIFKIAGQYDYWGQGTLVTVSS

SEQ ID NO: 54 - protein, 120 aa, synthetic construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSS

SEQ ID NO: 55 - protein, 117 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSS

SEQ ID NO: 56 - protein, 9 aa, synthetic construct
GGGGSGGGS

SEQ ID NO: 57 - protein, 251 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSHHHHHH

SEQ ID NO: 58 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLIPNLYNYLRKLDRALPDPIKIFEIGPCYRKESDGKEHLEEFTMLTFIQMGSGCTRENLESIITDFLNHLGIDFKIVGDSCMVLGDTLDVMHGDLELSSAVVGPIPLDREWGIDKPKIGAGFGLERLLKVKHDFKNIKRAARSESYYNGISTNL

SEQ ID NO: 59 - DNA, 1365 bp, synthetic construct
  source 1..1365: mol_type = other DNA
atggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaa

SEQ ID NO: 60 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 61 - protein, 275 aa, synthetic construct
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLGSVARDLRDHTDGPVKIFEMGSCFRKESHSGMHLEEFTMLNLFDMGPRGDATEVLKNYISVVMKAAGLPDYDLVQEESDVYKETIDVEINGQEVCSAAVGPTPIDAAHDVHEPWSGAGFGLERLLTIREKYSTVKKGGASISYLNGAKIN

SEQ ID NO: 62 - DNA, 45 bp, synthetic construct
  source 1..45: mol_type = other DNA
cttggctgttttggcggatgagagaagattttcagcctgatacag

SEQ ID NO: 63 - DNA, 60 bp, synthetic construct
  source 1..60: mol_type = other DNA
gtatatctccttcttaaagttaaacaaaattatttctagcccaaaaaaacgggtatggag

SEQ ID NO: 64 - protein, 297 aa, synthetic construct
  VARIANT 241: note = Fluorosulfonyltyrosine (FSY), fluorosulfonyloxybenzoyl-L-lysine (FSK), fluoro-fluorosulfonyltyrosine (FFY), or any derivative thereof
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGGSGGGGSGGGGSGGGGSQVQLQESGGGSVEAGGSLRLSCARSGWPYSTYSMNWFRQAPGKEREAVAGISSTMSGIIFAESKAGQFTISQDNAKNTVYLQMNNLKPEDTAIYYCAARRXFSLSSSSDDFDYWGQGTQVTVSSAAAYPYDVPDYGSSPSTPPTPSPSTPPHHHHHH

SEQ ID NO: 65 - protein, 5 aa, synthetic construct
GGGGS

(19) United States
(12) Patent Application Publication
(10) Pub. No.: US 2025/0353926 A1
(43) Pub. Date: Nov. 20, 2025

(54) CROSSLINKING MULTISPECIFIC ANTIBODIES

(71) Applicant: Enlaza Therapeutics, Inc., La Jolla, CA (US)

(72) Inventors: Sergio DURON, La Jolla, CA (US); Jason ROLAND, La Jolla, CA (US); Jerod PTACIN, La Jolla, CA (US); Analeah HEIDT, La Jolla, CA (US); Jun LIU, La Jolla, CA (US); Yorke ZHANG, La Jolla, CA (US)

(21) Appl. No.: 18/868,692

(22) PCT Filed: May 25, 2023
(86) PCT No.: PCT/US2023/067512

§ 371 Date: Nov. 22, 2024

Related U.S. Application Data
(60) Provisional application No. 63/346,798, filed on May 27, 2022.

(51) Int. Cl. C07K 16/30; A61P 35/00; C07K 16/28; C12N 9/00

(52) U.S. Cl. CPC C07K 16/30; A61P 35/00; C07K 16/2803; C07K 16/2809; C07K 16/283; C07K 16/2863; C07K 16/3069; C12N 9/93; C07K 2317/31; C07K 2317/569; C12Y 601/01026

(57) ABSTRACT

Provided herein are compositions and methods for cross-linking multi-specific antibodies to targets with conjugates. Further provided herein are conjugates comprising targeting domains, wherein the targeting domain comprises an unnatural amino acid. Further provided herein are methods of treating disease with cross-linking multi-specific antibodies.

### CROSS-REFERENCE

This application claims the benefit of U.S. Provisional Application No. 63/346,798, filed May 27, 2022, which application is incorporated herein in their entirety by reference.

### SEQUENCE LISTING

The instant application contains a Sequence Listing which has been submitted electronically in XML format and is hereby incorporated by reference in its entirety. Said XML copy, created on May 24, 2023, is named 60801-712_601_SL.xml and is 75 kilobytes in size.

### BACKGROUND

Proteins primarily use non-covalent interactions within or between proteins since amino acid side chains of proteins usually cannot form covalent bonds with each other, except for cysteine which generates relatively weak disulfide bonds that are reversible.

### BRIEF SUMMARY

Provided herein, in some aspects is a conjugate comprising: a first targeting domain configured to bind a first target on a first cell, and a second targeting domain configured to bind a second target on a second cell, wherein the first targeting domain comprises at least one first unnatural amino acid (UAA) whereby the first targeting domain is capable of covalently binding to a first target at the site of the UAA to the first target. In some embodiments, the second cell is an immune cell. In some embodiments, the first cell is a tumor cell. In some embodiments, the first targeting domain comprises an antibody or an antigen binding fragment thereof. In some embodiments, the first targeting domain comprises a single domain antibody (sdAb). In some embodiments, the first UAA is comprised within or within proximity of a region of the first targeting domain that interfaces with the first target. In some embodiments, the second targeting domain comprises an antibody or an antigen binding fragment thereof. In some embodiments, the second targeting domain comprises a single domain antibody (sdAb). In some embodiments, the first targeting domain and the second targeting domain are joined by chemical conjugation. In some embodiments, the first targeting domain and the second domain are joined by a linker. In some embodiments, the linker is a polypeptide linker. In some embodiments, the first target is a first cell surface molecule. In some embodiments, the second target is a second cell surface molecule. In some embodiments, the at least one UAA comprises an aryl-fluoro sulfate moiety. In some embodiments, the at least one first UAA comprises Formula I:

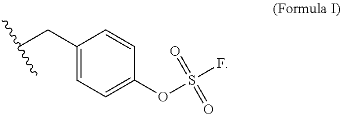 [SMILES: *Cc1ccc(OS(=O)(=O)F)cc1]

In some embodiments, the at least one first UAA has the structure:

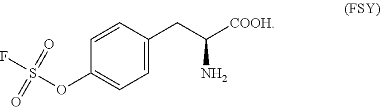 [SMILES: N[C@@H](Cc1ccc(OS(=O)(=O)F)cc1)C(=O)O]

In some embodiments, the at least one first UAA comprises Formula II:

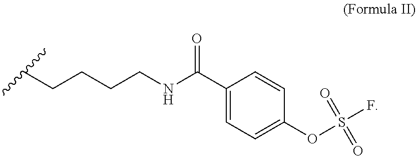 [SMILES: *CCCCNC(=O)c1ccc(OS(=O)(=O)F)cc1]

In some embodiments, the at least one first UAA has the structure:

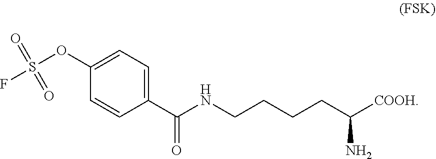 [SMILES: N[C@@H](CCCCNC(=O)c1ccc(OS(=O)(=O)F)cc1)C(=O)O]

In some embodiments, the at least one first UAA comprises Formula III:

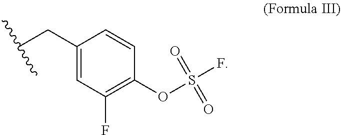 [SMILES: *Cc1ccc(OS(=O)(=O)F)c(F)c1]

In some embodiments, the at least one first UAA has the structure:

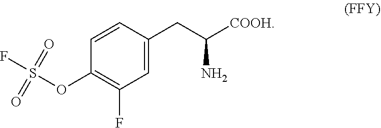 [SMILES: N[C@@H](Cc1ccc(OS(=O)(=O)F)c(F)c1)C(=O)O]

In some embodiments, the at least one first UAA has a structure of Formula (IA):

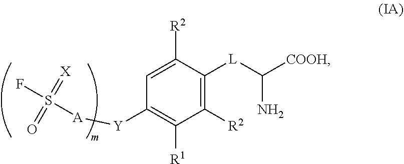

wherein, each X is independently O or NR′; Y is a bond, —O—, —NR—, or —N═; A is a bond or —(CH₂)ₙ—; m is 1 or 2; n is an integer from 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; L is —(CH₂)ₚ— or —C(O)NH—(CH₂)ₚ—; p is an integer from 1 to 6; and wherein when Y is —O— or —NR—, mis 1; when Y is —N═, m is 2. In some embodiments, the at least one first UAA has a structure of Formula (IA-a):

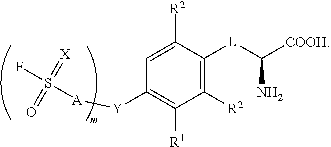

In some embodiments, the at least one first UAA has a structure of Formula (IB):

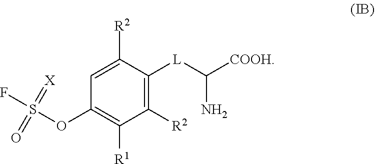

In some embodiments, the at least one first UAA has a structure of Formula (ID):

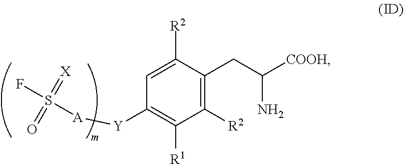

wherein: each X is independently O or NR′; Y is a bond, —O—, —NR—, or —N═; A is a bond or —(CH₂)ₙ—; m is 1 or 2; n is an integer from 1 to 4; each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl; R¹ is hydrogen, fluoro, or iodo; R² is hydrogen or methyl; and wherein when Y is a bond, —O— or —NR—, m is 1; when Y is —N═, m is 2. In some embodiments, the at least one first UAA has a structure of Formula (IIA):

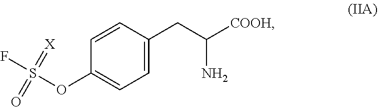

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or un substituted heterocycloalkyl. In some embodiments, the at least one first UAA has a structure of Formula (IIB):

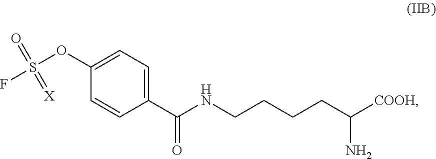

wherein: X is independently O or NR′; and R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, the first cell surface molecule comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some embodiments, the second cell surface molecule is a cell surface molecule present on an immune cell. In some embodiments, the immune cell comprises a T-cell or a NK-cell. In some embodiments, the second cell surface molecule is CD3, CD16, TCRαβ, NKD44, NKD46, NKD30, NKG2D, γδTCR, Vδ1, or Vγ9Vδ2. In some embodiments, the first targeting domain comprises any one of SEQ ID NOs: 1-4, 16-18, 20, 29, 50, or 51. In some embodiments, the first targeting domain comprises a sequence having at least 70% sequence identity to any one of SEQ ID NOs: 1-4, 16-18, 20, 29, 50, or 51. In some embodiments, the second targeting domain comprises a sequence having at least 70% sequence identity to SEQ ID NO: 22, 25, or 52-55. In some embodiments, the conjugate comprises a sequence having at least 70% sequence identity to any one of SEQ ID NOs: 19, 21, 23, 24, 26-28, 30, 35-49, 57, or 64. In some embodiments, the first targeting domain comprises SEQ ID NO: 16 or 51. In some embodiments, the first targeting domain comprising SEQ ID NO: 16 further comprises an unnatural amino acid at position 109 relative to SEQ ID NO: 16. In some embodiments, the first targeting domain comprising SEQ ID NO: 51 further comprises an unnatural amino acid at position 101 relative to SEQ ID NO: 51.

In some aspects, provided herein is a method comprising administering the conjugate according to embodiments of the disclosure, wherein the conjugate covalently binds the first target on the surface of a first cell. In some embodiments, the first cell is a tumor cell. In some embodiments, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some embodiments, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7.

In some aspects, provided herein is a method comprising administering the conjugate of according to embodiments of the disclosure, wherein the conjugate binds the second target on the surface of a second cell. In some embodiments, the first cell is a tumor cell. In some embodiments, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc 1, Muc16, NaPi2b, NECTIN4, UCHT1, VCAM-1, VEGF, VEGFR2, VpreB, VPREB1, or xCT. In some embodiments, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7.

In some aspects, provided herein is a method comprising administering the conjugate according to embodiments of the disclosure, wherein the conjugate covalently binds the first target on the surface of a first cell and the conjugate binds the second target on a second cell. In some embodiments, the first cell is a tumor cell. In some embodiments, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some embodiments, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some embodiments, the second cell is an immune cell, wherein the immune cell is a T-cell or a NK cell. In some embodiments the second cell comprises a second cell surface molecule. In some embodiments, the second cell surface molecule is CD3, CD16, TCRαβ, NKD44, NKD46, NKD30, NKG2D, γδTCR, Vδ1, or Vγ9Vδ2. In some embodiments, the first targeting domain comprises any one of SEQ ID NOs: 1-4, 16-18, 20, 29, 50, or 51. In some embodiments, the first targeting domain comprises a sequence having at least 70% sequence identity to any one of SEQ ID NOs: 1-4, 16-18, 20, 29, 50, or 51. In some embodiments, the second targeting domain comprises a sequence having at least 70% sequence identity to SEQ ID NO: 22, 25, or 52-55. In some embodiments, the conjugate comprises a sequence having at least 70% sequence identity to any one of SEQ ID NOs: 19, 21, 23, 24, 26-28, 30, 35-49, 57, or 64. In some embodiments, the first targeting domain comprises SEQ ID NO: 16 or 51. In some embodiments, the first targeting domain comprising SEQ ID NO: 16 further comprises an unnatural amino acid at position 109 relative to SEQ ID NO: 16. In some embodiments, the first targeting domain comprising SEQ ID NO: 51 further comprises an unnatural amino acid at position 101 relative to SEQ ID NO: 51.

In some aspects, provided herein is a method of treating a proliferative disease or disorder in a subject in need thereof, comprising administering to the subject a therapeutically effective amount of the conjugate according to embodiments of the disclosure. In some embodiments, the proliferative disease or condition is a cancer.

In some aspects, provided herein is a method of manufacturing the conjugate according to embodiments of the disclosure comprising synthesizing the first targeting domain comprising at least one unnatural amino acid in vivo. In some embodiments, the method further comprises synthesizing the second domain in vivo. In some embodiments, synthesizing comprises use of an orthogonal tRNA synthetase/suppressor tRNA pair. In some embodiments, synthesizing comprises the orthogonal tRNA synthetase/suppressor tRNA pair is derived from pyrrolysine tRNA synthetase/tRNA^{Pyl}.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts an SDS-PAGE analysis of crosslinking kinetics between EGFR and monoparatopic construct C4-109FSY. The t half-max crosslinking was approximately 120 minutes. Bands corresponding to the cross-linked product, EGFR, and sdAb monomer are labeled.

FIG. 1B depicts an SDS-PAGE analysis of crosslinking kinetics between EGFR and biparatopic construct C9 (C4-109FSY-L1-C₅). Bands corresponding to the cross-linked product, EGFR, and uncrosslinked C9 compound are labeled.

FIG. 2 depicts a plot of EGFR crosslinking kinetics for monoparatopic construct C4-109FSY (circles) or biparatopic construct C9 (C4-109FSY-L1-C5) (squares). The y-axis is labeled EGFR crosslinking %, from 0 to 100%. The x-axis is labeled Time (min) from 0 to 400 at 20 minute intervals.

FIG. 3 depicts exemplary arrangements of constructs provided herein. From top to bottom: C1 (wild type sequence comprising two cysteine disulfide linkages); C10 (C1-101A/104A) (one cysteine disulfide linkage is replaced with two alanines, removing the disulfide linkage); C1-102FSY (construct C1 with histidine 102 replaced with unnatural amino acid FSY); and C10-102FSY (C1-101A/104A/102FSY) (replacement of both C101 and C104 cysteines with alanine, and histidine 102 with FSY).

FIG. 4A depicts an SDS-PAGE analysis of construct C10-102FSY (C1-101A/104A/102FSY) monomer and C11-102FSY (C1-101A/104A/102FSY-L1-C10) dimer crosslinking with PSMA. Bands corresponding to the uncrosslinked C10-102FSY and C11-102FSY, PSMA, and crosslinked species are labeled. Constructs are depicted below, showing C10 monomer sequences (ovals), and FSY (triangles).

FIG. 4B depicts a plot of construct C10-102FSY (circles) and C11-102FSY (squares) crosslinking with PSMA.

FIG. 5A depicts exemplary constructs described herein comprising a first targeting domain (e.g., constructions C1-C4) comprising an unnatural amino acid (e.g., FSY), and a second targeting domain (e.g., Anti-CD3 [construct C6]; Anti-CD16 [construct C7]; Anti-NKD46; Anti-NKD30; Anti-TCRα; Anti-TCRβ, Anti-TCRγ; Anti-TCRδ).

FIG. 5B depicts an SDS-PAGE analysis of various constructs provided herein. Lanes 1-15 are labeled as follows: 1. C12 (C4-L6-C6); 2. C12+EGFR; 3. C13 (C4-109FSY-L6-C6); 4. C13+EGFR 5. C14 (C4-L2-C7); 6. C14+EGFR; 7. C15 (C4-109FSY-L2-C7); 8. C15+EGFR 9. C16 (C2-L6-C6); 10. C16+PSMA; 11. C18 (C2-54FSY-L6-C6); 12. C18+PSMA; 13. PSMA; 14. EGFR; 15. Ladder. Bands corresponding to EGFR, PSMA, and free sdAb dimer are labeled. Crosslinked species are marked with an asterisk.

FIG. 6 depicts an SDS-PAGE analysis of various constructs according to embodiments of the disclosure. Lanes 0 to 11 are labeled as follows: 0. Marker/Ladder; 1. C20 (C37-L2-C35) no PSMA; 2. C20 (C37-L2-C35) with PSMA; 3. C21 (C37-L1-C35) no PSMA; 4. C21 (C37-L1-C35) with PSMA; 5. C22 (C37-L3-C35) no PSMA; 6. C22 (C37-L3-C35) with PSMA; 7. C23 (C37-L4-C35) no PSMA: 8. C23 (C37-L4-C35) with PSMA; 9. C24 (C37-L5-C35) no PSMA; 10. C24 (C37-L5-C35) with PSMA; 11. PSMA. Crosslinked species, PSMA, uncrosslinked conjugates are labeled.

FIGS. 7A-7F illustrate binding and dissociation curves of constructs, according to embodiments of the disclosure, to PSMA.

FIG. 7A illustrates binding and dissociation curves of a negative control to PSMA.

FIG. 7B illustrates binding and dissociation curves of C21 to PSMA.

FIG. 7C illustrates binding and dissociation curves of C20 to PSMA.

FIG. 7D illustrates binding and dissociation curves of C22 to PSMA.

FIG. 7E illustrates binding and dissociation curves of C23 to PSMA at varying concentrations.

FIG. 7F illustrates binding and dissociation curves of C24 to PSMA at varying concentrations.

FIGS. 8A-8F illustrate binding and dissociation curves of constructs, according to embodiments of the disclosure, to CD16a.

FIG. 8A illustrates binding and dissociation curves of a control to CD16a.

FIG. 8B illustrates binding and dissociation curves of C21 to CD16a.

FIG. 8C illustrates binding and dissociation curves of C20 to CD16a.

FIG. 8D illustrates binding and dissociation curves of C22 to CD16a.

FIG. 8E illustrates binding and dissociation curves of C23 to CD16a at varying concentrations.

FIG. 8F illustrates binding and dissociation curves of C24 to CD16a at varying concentrations.

FIGS. 9A-9D illustrate binding and dissociation curves of constructs C22 and C25 to PSMA at pH 5.7 or pH 7.4.

FIG. 9A illustrates binding and dissociation curves of C25 to PSMA at varying concentrations in pH 5.7 buffer.

FIG. 9B illustrates binding and dissociation curves of C22 to PSMA at varying concentrations in pH 5.7 buffer.

FIG. 9C illustrates binding and dissociation curves of C25 to PSMA at varying concentrations in pH 7.4 buffer.

FIG. 9D illustrates binding and dissociation curves of C22 to PSMA at varying concentrations in pH 7.4 buffer, and the dissociation curve illustrates that at physiological pH (pH 7.4), the C22 construct containing FSY dissociated less than the C25 equivalent without FSY.

FIG. 10A illustrates a graph of binding and dissociation curves of C37 (antiCD16a-sd Ab) to CD16a at varying concentrations.

FIG. 10B illustrates a graph of binding and dissociation curves of C25 (antiCD16a-sdAb) to CD16a at varying concentrations.

FIG. 10C illustrates a graph of binding and dissociation curves of C22 (antiCD16a-sdAb) to CD16a at varying concentrations.

FIG. 11A depicts a graph with a y-axis corresponding to relative light units (RLU) versus an x-axis corresponding to test article concentration (nM). The test articles included J591m Ab, C25 (TYR), C22 (FSY), hIgG isotype (control), and C37, C36, C35 and were incubated with high PSMA density target cells (PC3pip cells) for 24 hours. The results indicate that C22 (FSY) (open square) was able to bind to more PSMA than C25 (TYR)

FIG. 11B, depicts a graph with a y-axis corresponding to relative light units (RLU) versus an x-axis correspond to test article concentration (nM). The test articles included J591mAb, C25 (TYR), C22 (FSY), hIgG isotype (control), and C37 and were incubated with high PSMA density target cells (PC3pip cells) for 24 hours with a wash step after 4 hours. The results indicate that C22 (FSY) (open square) was able to bind to PSMA.

FIG. 12A depicts a graph with a y-axis corresponding to relative light units (RLU) versus an x-axis correspond to test article concentration (nM). The test articles included J591mAb, C25 (TYR), C22 (FSY), hIgG isotype (control), C37, C36, C35, and endotoxin control and were incubated with low PSMA density target cells (22Rv1 cells) for 24 hours. The results indicate that C22 (FSY) (open square) and C25 (TYR) (open dark circle) were able to bind to PSMA.

FIG. 12B depicts a graph with a y-axis corresponding to cell lysis in percent (%) versus an x-axis correspond to test article concentration (nM). The conjugates tested included C25 (TYR), C22 (FSY), J591mAb and were incubated with low PSMA density target cells (22Rv1 cells) for 24 hours with a wash step after 4 hours. The results illustrate that C22 (FSY) was able to covalently bind to the low PSMA density target cells, as evidenced by an increase in RLU.

FIG. 13A depicts a graph with a y-axis corresponding to cell lysis in percent (%) versus an x-axis corresponding to test article concentration (nM). The conjugates tested included C22, C25, C37 and were incubated with primary NK cells for 20 hours.

FIG. 13B depicts a graph with a y-axis corresponding to cell lysis in percent (%) versus an x-axis corresponding to test article concentration (nM). The conjugates tested included C22, C25, C37 and were incubated with primary NK cells for 20 hours with a wash step after 5 hours.

FIG. 14A depicts an SDS-PAGE of constructs (or conjugates) in the presence of PSMA. The constructs, C27 (FSY) and C26 (TYR), were mixed with PSMA, and aliquots at different time intervals (0, 5, 10, and 15 minutes) were sampled and analyzed by SDS-PAGE in the presence (R) or absence (NR) of receptor. The stars indicated shifted species (*), un-shifted receptor (**), and non-crosslinked C26 or C27 (***). The results indicate that C27 (FSY) was able to covalently bind to the PSMA target.

FIG. 14B depicts an SDS-PAGE of construct C28 (TYR) in the presence of PSMA. The construct C28 (TYR) was mixed with PSMA, and aliquots at different time intervals (0, 5, 10, and 15 minutes) were sampled and analyzed by SDS-PAGE in the presence (R) or absence (NR) of receptor. The stars indicated un-shifted receptor (*), and non-crosslinked C28 (**). The results indicate that C28 (TYR) was unable to covalently bind to the PSMA target.

FIG. 14C depicts an SDS-PAGE of constructs in the presence of PSMA. The constructs, C29 (TYR) and C30 (FSY), were mixed with PSMA, and aliquots at different time intervals (0, 5, 10, and 15 minutes) were sampled and analyzed by SDS-PAGE in the presence (R) or absence (NR) of receptor. The stars indicated shifted species (*), un-shifted receptor (**), and non-crosslinked C29 or C30 (***). The results indicate that C30 (FSY) was able to covalently bind to the PSMA target.

FIG. 14D depicts an SDS-PAGE of constructs in the presence of PSMA. The constructs, C31 (TYR) and C32 (TYR), were mixed with PSMA, and aliquots at different time intervals (0, 5, 10, and 15 minutes) were sampled and analyzed by SDS-PAGE in the presence (R) or absence (NR) of receptor. The stars indicated un-shifted receptor (*), and non-crosslinked C31 or C32 (**). The results indicate that C31 (TYR) and C32 (TYR) were unable to covalently bind to the PSMA target.

FIG. 14E depicts an SDS-PAGE of constructs in the presence of PSMA. The constructs, C34 (FSY) and C33 (TYR), were mixed with PSMA, and aliquots at different time intervals (0, 5, 10, and 15 minutes) were sampled and analyzed by SDS-PAGE in the presence (R) or absence (NR) of receptor. The stars indicated shifted species (*), un-shifted receptor (**), and non-crosslinked C33 or C34 (***). The results indicate that C34 (FSY) was able to covalently bind to the PSMA target.

FIG. 15 depicts a graph of a T-cell ADCC luciferase reporter assay, and the y-axis corresponds to relative light units (RLUs) versus the x-axis corresponding to concentration (nM) of the conjugates according to embodiments of the disclosure.

FIGS. 16A-16C depict three graphs of T-cell ADCC luciferase reporter assays, and the y-axes correspond to relative light units (RLUs) versus x-axes corresponding to concentration (nM) of the conjugates according to embodiments of the disclosure, a commercially available CD3-PSMA bispecific, and an isotype (control).

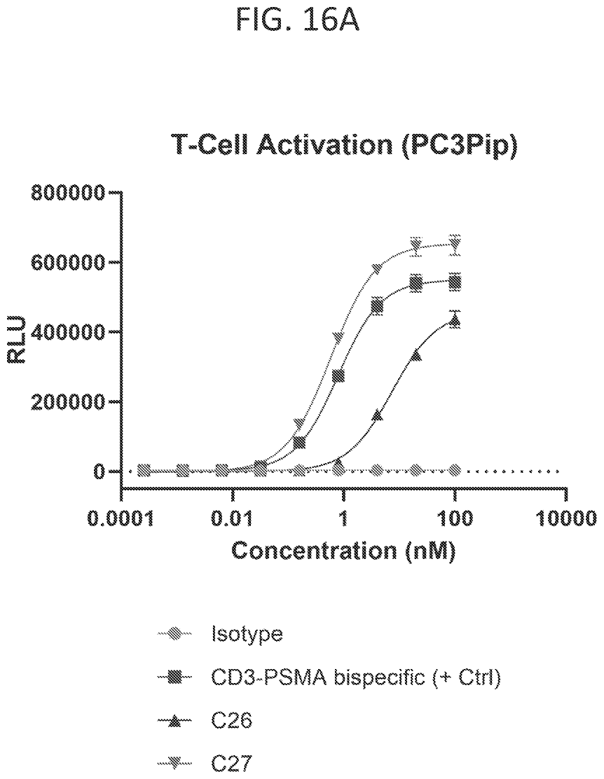

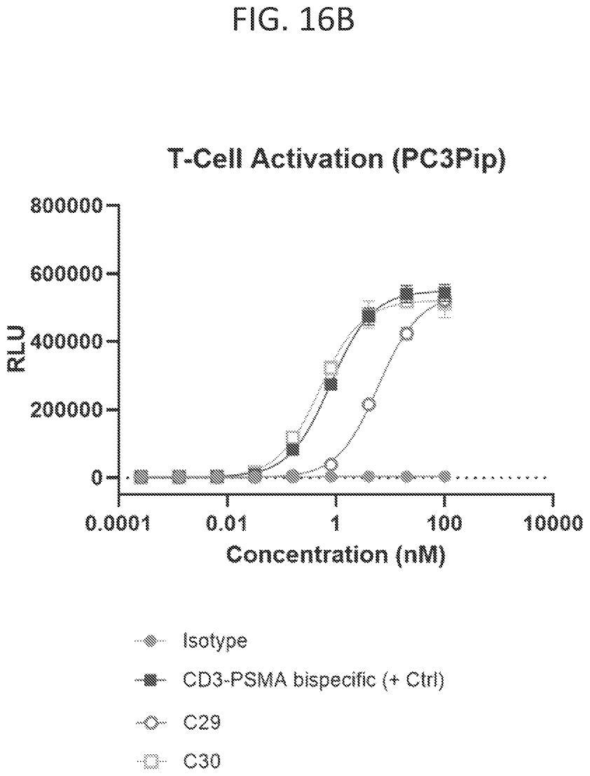

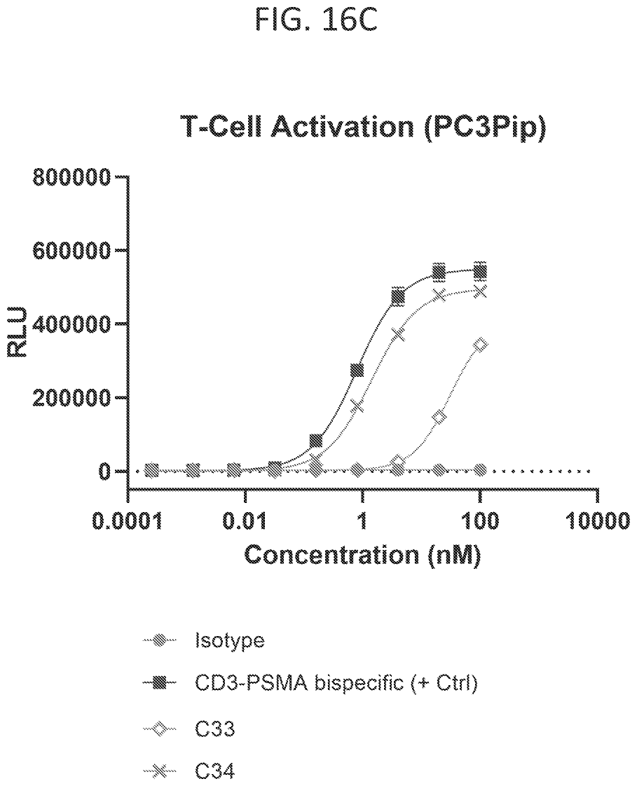

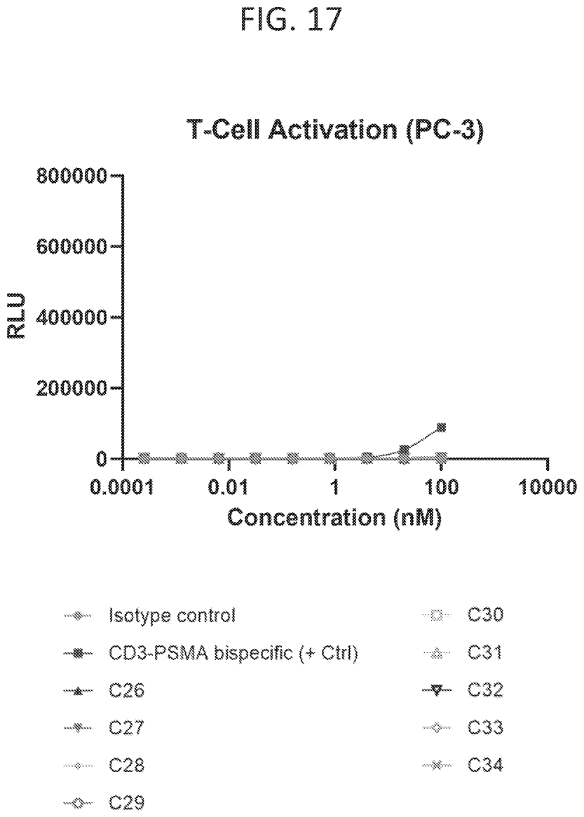

In FIG. 16A, a comparison of binding curves for C27 (FSY), C26 (TYR), and commercially available CD3-PSMA bispecific, and an isotype control illustrates that C27 bound to PSMA-expressing PC3 Pip target cells with a smaller EC50 than C26. In FIG. 16B, a comparison of binding curves for C30 (FSY), C29 (TYR), and commercially available CD3-PSMA bispecific, and an isotype control illustrates that C30 bound to PSMA-expressing PC3 Pip target cells with a smaller EC50 than C29. In FIG. 16C, a comparison of binding curves for C33 (FSY), C34 (TYR), and commercially available CD3-PSMA bispecific, and an isotype control illustrates that C34 bound to PSMA-expressing PC3Pip target cells with a smaller EC50 than C33.

FIG. 17 depicts a graph of T-cell activation in PC3Pip cells, where the graph illustrates a y-axis corresponding to relative light units (RLUs) versus an x-axis corresponding to concentration (nM) of the conjugates according to embodiments of the disclosure, a commercially available CD3-PSMA bispecific, and an isotype (control). The graph illustrates that the various constructs according to embodiments of the disclosure do not activate T cells in the absence of PSMA expression on the target cell.

### DETAILED DESCRIPTION

Antibodies, antibody fragments and antibody-related constructs can be useful tools for research and clinical applications. However, in some instances use of these molecules is limited by on/off rates for a target, as well as stability. Provided herein are compositions and methods for specific and covalent binding of conjugates to targets. In some instances, a conjugate comprises a first targeting domain, and a second targeting domain, and at least one unnatural amino acid (UAA), wherein the first targeting domain is configured to bind to a first target on a first cell and the second targeting domain is configured to bind to a second target on a second cell. In some instances, the first cell is a tumor cell, and the second cell is an immune cell. In some instances, the second targeting domain is configured to bind to protein on the surface of the immune cell and the immune cell is a T-cell, a NK cell, a myeloid cell or a macrophage. In some cases, the T-cell is a NKT-cell. In some instances, the first or second targeting domain comprises at least one UAA that is present in the proximity of the interface between the target and the targeting moiety, such that when the targeting domain and target are bound, a covalent bond is formed between target and the UAA. In some instances, covalent interactions eliminate or reduce off rates of conjugates binding to targets, or otherwise stabilize contact to the target. In some cases, the second targeting domain can also include a UAA. In some cases, only the first targeting domain, but not the second domain, includes a UAA. In some cases, only the first targeting domain includes a UAA and the first targeting domain is configured to bind to a first target on a tumor cell. In some cases, only the first targeting domain includes a UAA and the first targeting domain is configured to bind to a first target on a tumor cell and the second targeting domain is configured to bind to a second target on an immune cell.

### Conjugates

Conjugates herein include a first targeting domain, and a second targeting domain, wherein the first targeting domain is configured to bind to a first target, such as a first target on a tumor cell, and the second targeting domain is configured to bind to a second target that is present on a different cell. In some instances, the second target is on an immune cell such as a T-cell, NK cell, myeloid cell or macrophage. In some instances, the conjugate comprises at least one unnatural amino acid (UAA) comprised in the first targeting domain, in the second targeting domain or each of the first targeting domain and the second targeting domain comprises at least one UAA, such that the targeting domain with the UAA is configured to bind to its target and one of the UAAs within the targeting domain forms a covalent bond with the target. In some instances, the first targeting domain is configured to form a covalent bond with a target, such that at least one UAA is present in the targeting domain in the proximity of the interface between the target and the targeting domain, such that when the targeting domain is bound to or in proximity to the target, a covalent bond is formed between the target and the UAA in the targeting domain of the conjugate.

In some instances, when the UAA comprised in the first targeting domain forms a covalent bond with its target (e.g., a target on the surface of a tumor cell), and the second targeting domain in the conjugate binds to a second target (e.g., specifically binds the second target, or binds by the UAA forming a covalent bond with the second target) that is on an immune cell such as T-cell, NK cell, myeloid cell or macrophage, the conjugate brings the immune cell (e.g., T-cell, NK cell, myeloid cell or macrophage) in proximity to the tumor cell, and accordingly activates the immune system against the tumor cell. In one embodiment, the immune cell is a T-cell, and the second targeting domain binds to a second target on a T-cell. In another embodiment, the immune cell is a NK cell, and the second targeting domain binds to a second target on a NK cell. In some cases, the T-cell is a NKT-cell. In yet another embodiment, the immune cell is a myeloid cell, and the second targeting domain binds to a second target on a myeloid cell. In yet another embodiment, the immune cell is a macrophage, and the second targeting domain binds to a second target on a macrophage.

In some instances, a conjugate comprises more than two targeting domains, and has at least 1, 2, 3, 4, 5, 6, or more than 7 targeting domains. In some instances, targeting domains are attached to each other via linker (e.g., chemical linker, fusion protein, or other linker provided herein). In some instances, the first targeting domain and the second targeting domain of the conjugates herein are joined as a fusion protein.

In some instances, a targeting domain (e.g., the first targeting domain, the second targeting domain or both) comprises an antibody or antigen binding fragment, such as a monospecific Fab2, bispecific Fab2, trispecific Fab3, monovalent IgG, scFv, bispecific diabody, trispecific triabody, scFv-Fc, sdAb, minibody, IgNAR, V-NAR, hcIgG, VHH, or peptibody. In some embodiments, the targeting moiety comprises a single domain antibody (sdAb; also referred to as a nanobody). In some instances, a targeting domain comprises one or more complementarity determining regions (CDR) regions. In some embodiments, the targeting domain comprises an antibody or antigen binding fragment that binds to a cell surface molecule. In some instances, a conjugate comprises a second targeting domain and the second targeting domain comprises an antibody or antigen binding fragment such as a monospecific Fab2, bispecific Fab2, trispecific Fab3, monovalent IgG, scFv, bispecific diabody, trispecific triabody, scFv-Fc, sdAb, minibody, IgNAR, V-NAR, hcIgG, VHH, or peptibody. In some embodiments, the first targeting domain and the second targeting domain each comprise an antibody or antigen binding fragment, and the structure of such antibody or antigen binding fragment may be the same or different. For example, the first targeting domain can be a single chain antibody (e.g., Fv) and the second targeting domain can be a sdAb, or for example, the first targeting domain can be a sdAb and the second targeting domain can be a sdAb.

In some instances, the targeting domain (e.g., the first targeting domain and/or the second targeting domain) is an antibody mimic such as an affibody, Darpin or a mini binder.

Unnatural amino acids may be located at any position in the conjugate. In some instances, the first targeting domain comprises an unnatural amino acid. In some instances, the first targeting domain is an antibody or antigen binding fragment comprising one or more complementary determining regions (CDRs) and the one or more unnatural amino acids is comprised within or within proximity of a CDR in the first targeting domain. In some instances, the second targeting domain comprises an unnatural amino acid. In some instances, the second targeting domain is an antibody or antigen binding fragment comprising one or more complementary determining regions (CDRs) and the one or more unnatural amino acids is comprised within or within proximity of a CDR in the second targeting domain.

In some instances, a targeting domain comprises any one of SEQ ID NOs: 1-4, 16-18, 20, 22, 29, 50, or 51. In some instances, a targeting domain comprises a sequence having at least 99%, 98%, 97%, 95%, 90%, 85%, 80%, 70%, or at least 65% sequence identity to any one of SEQ ID NOs: 1-4, 16-18, 20, 22, 25, 29, 50, or 51. In some instances, a targeting domain comprises a sequence having at least 99%, 98%, 97%, 95%, 90%, 85%, 80%, 70%, or at least 65% sequence identity to any one of SEQ ID NOs: 1-4, 16-18, 20, 22, 25, 29, or 50-55, and at least one unnatural amino acid. In some instances, the targeting domain comprises construct C1, C2, C3, C4, C5, C6, C7, C10, C11, C17, C35, C36, C37, C38, C39, or C40. In exemplary arrangement, a conjugate comprises at least two targeting domains (e.g., a first targeting domain and a second targeting domain). In some instances, a conjugate comprises at least two targeting domains selected from the group consisting of construct C1, C2, C3, C4, C5, C6, C7, C10, C11, C17, C35, C36, C37, C38, C39, and C40. In some instances, a conjugate comprises a first targeting domain selected from the group consisting of construct C1, C2, C3, C4, C5, C10, C11, C17, C35, C36, C37, C38, C39, and C40 and a second targeting domain. In some cases, the second targeting domain comprises C6, C7, C37, C38, C39, and C40. In some instances, a conjugate comprises a second targeting domain of C6, C7, C37, C38, C39, and C40, and also comprises a first targeting domain that binds to a cell surface protein on a tumor cell. In some instances, the first targeting domain and the second targeting domain are the same. In some instances, the first targeting domain and the second targeting domain are different.

In some instances, the conjugate comprises a tag, such as for purification (e.g., a his6 tag). In some cases, the tag comprises a polyhistidine tag (e.g., his3, his4, his5, his6, his7, his8, or his9), hemagglutinin (HA) tag, an SP tag (SEQ ID NO: 34), or a combination thereof. In some instances, a conjugate does not comprise a his6 tag, or the tag is removed prior to administration. In some instances, the conjugate comprises a leader sequence such as for expression or secretion. In some instances, a conjugate does not comprise a leader sequence, or the leader sequence is removed prior to administration.

In some instances, the conjugate comprises a signal sequence. The signal sequence may allow for expression, folding, or oxidation of the conjugate in a bacterial cell. The signal sequence may allow for the expressed conjugate in a bacterial to be transported to another location or environment to promote the folding or function of the conjugate. The signal sequence may be a PelB sequence. The signal sequence may allow the transport of the conjugate to the periplasm of a bacterial cell. The environment may be oxidizing or reducing such to allow for the formation of disulfide or the reduction of disulfides.

### Targets

Conjugates provided herein may be configured to bind to one or more targets. In some instances, first and second targeting domains bind to the one or more targets. In some instances, a UAA comprised in a targeting domain forms a covalent bond between the targeting domain and the target. In some instances, a conjugate comprises a first targeting domain comprising a first UAA and second targeting domain comprising a second UAA. Such UAAs are in some instances the same or in other instances are different. In some instances, a target is a cell surface molecule (i.e., present in whole or in part on the outer surface of a cell). In some instances, a first targeting domain and a second targeting domain each bind to different cell surface molecules.

In some instances, the first targeting domain of the conjugate is configured to bind to a first target, and the first target is a cell surface molecule present on a tumor cell. In some cases, the first target comprises a first cell surface molecule. A target in some instances is a monomer. In some instances, the first target is comprised in a multimeric structure of homogenous or heterogenous units. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrina10B1, ITGaVb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor 1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, Eph A2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc 1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a cytokine. In some instances, the cytokine comprises CD2, CD13, CD19, CD20, CD22, CD24, CD30, CD33, CD34, CD36, CD38, CD44v6, CD45, CD46, CD47, CD48, CD56, CD66, CD70, CD71, CD73, CD74, CD79b, CD99, CD123, CD138, CD142, CD147, CD155, CD166, CD171, CD205, CD228, or CD248.

In some instances, the second targeting domain of the conjugate is configured to bind to a second target and the second target is a cell surface molecule present on an immune cell. In some instances, the immune cell is a T-cell, an NK-cell, a gamma-delta T cell, a macrophage, a myeloid cell or other immune cell. In some instances, the T-cell is a NKT-cell. In some instances, the second target comprises CD3, CD16, TCRαβ, NKD44, NKD46, NKD30, NKG2D, γδTCR, Vδ1, Vγ9Vδ2, or PD-L1.

In some instances, a first targeting domain binds with a first target of a first cell, and a second targeting domain binds to a second target of a second cell and the first cell is a tumor cell and the second cell is an immune cell. In some instances, a first target on the first cell comprises one or more of 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, Eph A2, Eph A3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, orxCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB 1. In some instances, the second target on the second cell comprises one or more of CD3, CD16, TCRαβ, NKD44, NKD46, NKD30, NKG2D, YOTCR, Vδ1, Vγ9Vδ2, or PD-L1.

Targets (e.g., first target, second target or both) that are engaged by targeting domains (e.g., first targeting domain, second targeting domain or both) may comprise a variety of structures. In some instances, a target comprises one or more epitopes that can be engaged by a targeting domain. In some instances, a target comprises multiple epitopes and can be engaged by multiple targeting domains. In some instances, a target comprises a monomer. In some instances, a target comprises a single chain peptide. In some instances, a target comprises a multimeric molecule. In some instances, a multimeric molecule comprises two or more sub-units. In some instances, sub-units have the same structure. In some instances, sub-units are different structures, or a combination of the same and different structures. In some instances, a multimeric molecule comprises two or more molecules in a complex. In some instances, a multimeric molecule comprises two proteins that complex or interact with each other.

In some embodiments, the conjugate may bind to a first target. In some instances, the conjugate comprises a first targeting domain configured to bind to a first target on a first cell. In some instances, the first targeting domain binds to the first target via ionic bonds, electrostatic interactions, hydrogen bonding, van der Waals forces, pi-pi stacking, or a combination thereof. In some embodiments, the first targeting component forms a covalent bond (i.e., covalently crosslinks or covalently binds to) with the first target. In some instances, the first targeting component forms a covalent bond with the first target when the first targeting component comprises at least one UAA.

In some embodiments, the conjugate may bind to a second target. In some instances, the conjugate comprises a second targeting domain configured to bind to a second target on a second cell. In some instances, the second targeting domain binds to the second target via ionic bonds, electrostatic interactions, hydrogen bonding, van der Waals forces, pi-pi stacking, or a combination thereof. In some embodiments, the second targeting component forms a covalent bond (i.e., covalently crosslinks or covalently binds to) with the second target. In some instances, the second targeting component forms a covalent bond with the second target when the second targeting component comprises at least one UAA.

In some embodiments, the binding of the conjugate component to a target may be characterized by an affinity constant (K_{A}), which may be expressed as a dissociation constant (K_{D}). In some instances, the conjugate comprises a first targeting domain configured to bind to a first target on a first cell. In some instances, the binding of the first targeting domain to the first target may be characterized by K_{D1}. In some instances, K_{D1} ranges from about 1 pM to about 100 mM, from 10 nM to about 1 mM, from about 100 nM to about 50 M, from about 50 nM to about 100 μM, from about 1 μM to about 100 μM. In some instances, K_{D1} ranges from about 1 nM to about 1 μM, from 10 nM to about 500 nM, from about 50 nM to about 250 nM, from about 100 nM to about 500 nM, or from about 1 nM to about 100 nM. In some instances, K_{D1} is at least about 1 nM, about 10 nM, about 25 nM, 50 nM, about 100 nM, about 250 nM, about 500 nM, about 1 μM, about 50 μM, about 100 μM, about 250 μM, about 500 μM, or about 1 mM. In some instances, when the first targeting domain comprises at least one UAA, the at least one UAA may covalently bind to the first target. In some cases, when the at least one UAA covalently binds to the first target, K_{D1} is about 0 pM, about 10 pM, about 20 pM, about 50 pM, or about 100 pM.

In some embodiments, the binding of the conjugate component to a target may be characterized by an affinity constant (K_{A}), which may be expressed as a dissociation constant (K_{D}). In some instances, the conjugate comprises a second targeting domain configured to bind to a second target on a first cell. In some instances, the binding of the second targeting domain configured to bind to a second target may be characterized by K_{D2}. In some instances, K_{D2} ranges from about 1 pM to about 100 mM, from 10 nM to about 1 mM, from about 100 nM to about 50 μM, from about 50 nM to about 100 μM, from about 1 μM to about 100 μM. In some instances, K_{D2} ranges from about 1 nM to about 1 μM, from 10 nM to about 500 nM, from about 50 nM to about 250 nM, from about 100 nM to about 500 nM, or from about 1 nM to about 100 nM. In some instances, K_{D2} is at least about 1 nM, about 10 nM, about 25 nM, 50 nM, about 100 nM, about 250 nM, about 500 nM, about 1 μM, about 50 μM, about 100 μM, about 250 μM, about 500 μM, or about 1 mM. In some instances, when the first targeting domain comprises at least one UAA, the at least one UAA may covalently binds to the first target. In some cases, when the at least one UAA covalently binds to the first target, K_{D2} is about 0 pM, about 10 pM, about 20 pM, about 50 pM, or about 100 pM.

In some embodiments, the binding of the conjugate component to a target may be characterized by a rate of binding to the target (kₒₙ). In some instances, the conjugate comprises a first targeting domain configured to bind to a first target on a first cell. In some instances, the binding of the first targeting domain to the first target may be characterized by a rate of binding k_{on,1}. In some instances, k_{on,1} is determined by an enzyme kinetics measurement. In some instances, the enzyme kinetics measurement comprises biolayer interferometry (BLI), surface plasmon resonance (SPR), or the like. In some instances, k_{on,1} is monophasic. In some instances, k_{on,1} is biphasic. In some instances, k_{on,1} ranges from about 1×10¹ M⁻¹ s⁻¹ to about 1×10⁶ M⁻¹ s⁻¹, from about 5×10¹ M⁻¹ s⁻¹ to about 1×10⁵ M⁻¹ s⁻¹, from about 1×10² M⁻¹ s⁻¹ to about 5×10⁴ M⁻¹ s⁻¹, or from about 5×10² M⁻¹ s⁻¹ to about 1×10³ M⁻¹ s⁻¹. In some instances, k_{on,1} is at least about 1×10¹ M⁻¹ s⁻¹, about 2×10¹ M⁻¹ s⁻¹, about 5×10¹ M⁻¹ s⁻¹, 1×10¹ M⁻¹ s⁻¹, about 2×10¹ M⁻¹ s⁻¹, about 5×10¹ M⁻¹ s⁻¹, about 1×10² M⁻¹ s⁻¹, about 2×10² M⁻¹ s⁻¹, about 5×10² M⁻¹ s⁻¹, about 1×10³ about M⁻¹ s⁻¹, about 2×10³ M⁻¹ s⁻¹, about 5×10³ M⁻¹ s⁻¹, about 1×10⁴ M⁻¹ s⁻¹, about 2×10⁴ M⁻¹ s⁻¹, about 5×10⁴ M⁻¹ s⁻¹, about 1×10⁵ M⁻¹ s⁻¹, about 2×10⁵ M⁻¹ s⁻¹, about 5×10⁵ M⁻¹ s⁻¹, or about 1×10⁶ M⁻¹ s⁻¹, about 2×10⁶ M⁻¹ s⁻¹, about 5×10⁶ M⁻¹ s⁻¹. In some instances, k_{on,1} is at most about 1×10⁴ M⁻¹ s⁻¹, about 2×10⁴ M⁻¹ s⁻¹, about 5×10⁴ M⁻¹ s⁻¹, about 1×10⁵ M⁻¹ s⁻¹, about 2×10⁵ M⁻¹ s⁻¹, about 5×10⁵ M⁻¹ s⁻¹, or about 1×10⁶ M⁻¹ s⁻¹, about 2×10⁶ M⁻¹ s⁻¹, about 5×10⁶ M⁻¹ s⁻¹.

In some embodiments, the binding of the conjugate component to a target may be characterized by a rate of binding to the target (kₒₙ). In some instances, the conjugate comprises a second targeting domain configured to bind to a second target on a second cell. In some instances, the binding of the second targeting domain to the second target may be characterized by a rate of binding k_{on,2}. In some instances, k_{on,2} is determined by an enzyme kinetics measurement. In some instances, the enzyme kinetics measurement comprises biolayer interferometry (BLI), surface plasmon resonance (SPR), or the like. In some instances, k_{on,2} is monophasic. In some instances, k_{on,2} is biphasic. In some instances, k_{on,2} ranges from about 1×10¹ M⁻¹ s⁻¹ to about 1×10⁶ M⁻¹ s⁻¹, from about 5×10¹ M⁻¹ s⁻¹ to about 1×10⁵ M⁻¹ s⁻¹, from about 1×10² M⁻¹ s⁻¹ to about 5×10⁴ M⁻¹ s⁻¹, or from about 5×10² M⁻¹ s⁻¹ to about 1×10³ M⁻¹ s⁻¹. In some instances, k_{on,2} is at least about 1×10¹ M⁻¹ s⁻¹, about 2×10¹ M⁻¹ s⁻¹, about 5×10¹ M⁻¹ s⁻¹, 1×10¹ M⁻¹ s⁻¹, about 2×10¹ M⁻¹ s⁻¹, about 5×10¹ M⁻¹ s⁻¹, about 1×10² M⁻¹ s⁻¹, about 2×10² M⁻¹ s⁻¹, about 5×10² M⁻¹ s⁻¹, about 1×10³ about M⁻¹ s⁻¹, about 2×10³ M⁻¹ s⁻¹, about 5×10³ M⁻¹ s⁻¹, about 1×10+M⁻¹ s⁻¹, about 2×10⁴ M⁻¹ s⁻¹, about 5×10⁴ M⁻¹ s⁻¹, about 1×10⁵ M⁻¹ s⁻¹, about 2× 10⁵ M⁻¹ s⁻¹, about 5×10⁵ M⁻¹ s⁻¹, or about 1×10⁶ M⁻¹ s⁻¹, about 2×10⁶ M⁻¹ s⁻¹, about 5×10⁶ M⁻¹ s⁻¹. In some instances, k_{on,2} is at most about 1×10⁴ M⁻¹ s⁻¹, about 2×10⁴ M⁻¹ s⁻¹, about 5×10⁴ M⁻¹ s⁻¹, about 1×10⁵ M⁻¹ s⁻¹, about 2×10⁵ M⁻¹ s⁻¹, about 5×10⁵ M⁻¹ s⁻¹, or about 1×10⁶ M⁻¹ s⁻¹, about 2×10⁶ M⁻¹ s⁻¹, about 5×10⁶ M⁻¹ s⁻¹.

In some embodiments, the binding of the conjugate component to a target may be characterized by a dissociation rate, or “off-rate”, which can be expressed as k_{off}. In some instances, the conjugate comprises a first targeting domain configured to bind to a first target on a first cell. In some instances, the binding of the first targeting domain to the first target may be characterized by a rate of binding k_{off,1}. In some instances, k_{off,1} is determined by an enzyme kinetics measurement. In some instances, the enzyme kinetics measurement comprises biolayer interferometry (BLI), surface plasmon resonance (SPR), or the like. In some instances, k_{off,1} is monophasic. In some instances, k_{off,1} is biphasic. In some instances, k_{off,1} ranges from about 1×10⁻⁹ s⁻¹ to about 1×10⁻² M⁻¹ s⁻¹, from about 1×10⁻⁸ s⁻¹ to about 1×10⁻³ M⁻¹ s⁻¹, or from about 1×10⁻⁷ s⁻¹ to about 1×10⁻⁴ M⁻¹ s⁻¹. In some instances, k_{off,1} is at least about 1×10⁻⁹ s⁻¹, about 2×10⁻⁹ s⁻¹, about 5×10⁻⁹ s⁻¹, about 1×10⁻⁸ s⁻¹, about 2×10⁻⁸ s⁻¹, about 5×10⁻⁸ s⁻¹, about 1×10⁻⁷ s⁻¹, about 2×10⁻⁷ s⁻¹, about 5×10⁻⁷ s⁻¹, about 1×10⁶ s⁻¹, about 2×10⁶ s⁻¹, about 5×10⁶ s⁻¹, about 1×10⁻⁵ s⁻¹, about 2×10⁻⁵ s⁻¹, about 5×10⁻⁵ s⁻¹, about 1×10⁻⁴ s⁻¹, about 2×10⁻⁴ s⁻¹, about 5×10⁻¹ s⁻¹, about 1×10⁻³ s⁻¹, about 2×10⁻³ s⁻¹, about 5×10⁻³ s⁻¹, about 1×10⁻² s⁻¹, about 2×10⁻² s⁻¹, about 5×10⁻² s⁻¹, or about 1×10⁻¹ s⁻¹, about 2×10⁻¹ s⁻¹, about 5×10⁻¹ s⁻¹. In some instances, k_{off,1} is at most about 1×10⁻⁴ s⁻¹, about 2×10⁻⁴ s⁻¹, about 5×10⁴ s⁻¹, about 1×10⁻³ s⁻¹, about 2×10⁻³ s⁻¹, about 5×10⁻³ s⁻¹, about 1×10⁻² s⁻¹, about 2×10⁻² s⁻¹, about 5×10⁻² s⁻¹, or about 1×10⁻¹ s⁻¹, about 2×10⁻¹ s⁻¹, about 5×10⁻¹ s⁻¹.

In some embodiments, the binding of the conjugate component to a target may be characterized by a dissociation rate, or “off-rate”, which can be expressed as k_{off}. In some instances, the conjugate comprises a second targeting domain configured to bind to a second target on a second cell. In some instances, the binding of the second targeting domain to the second target may be characterized by a rate of binding k_{off,2}. In some instances, k_{off,2} is determined by an enzyme kinetics measurement. In some instances, the enzyme kinetics measurement comprises biolayer interferometry (BLI), surface plasmon resonance (SPR), or the like. In some instances, k_{off,2} is monophasic. In some instances, k_{off,2} is biphasic. In some instances, k_{off,2} ranges from about 1×10⁻⁹ s⁻¹ to about 1×10⁻² M⁻¹ s⁻¹, from about 1×10⁻⁸ s⁻¹ to about 1×10⁻³ M⁻¹ s⁻¹, or from about 1×10⁻⁷ s⁻¹ to about 1×10⁻¹ M⁻¹ s⁻¹. In some instances, k_{off,2} is at least about 1×10⁻⁹ s⁻¹, about 2×10⁻⁹ s⁻¹, about 5×10⁻⁹ s⁻¹, about 1×10⁻⁸ s⁻¹, about 2×10⁻⁸ s⁻¹, about 5×10⁻⁸ s⁻¹, about 1×10⁻⁷ s⁻¹, about 2×10⁻⁷ s⁻¹, about 5×10⁻⁷ s⁻¹, about 1×10⁶ s⁻¹, about 2×10⁻⁶ s⁻¹, about 5×10⁶ s⁻¹, about 1×10⁻⁵ s⁻¹, about 2×10⁻⁵ s⁻¹, about 5×10⁻⁵ s⁻¹, about 1×10⁴ s⁻¹, about 2×10⁴ s⁻¹, about 5×10⁻⁴ s⁻¹, about 1×10⁻³ s⁻¹, about 2×10⁻³ s⁻¹, about 5×10⁻³ s⁻¹, about 1×10⁻² s⁻¹, about 2×10⁻² s⁻¹, about 5×10⁻² s⁻¹, or about 1×10⁻¹ s⁻¹, about 2×10⁻¹ s⁻¹, about 5×10⁻¹ s⁻¹. In some instances, k_{off,2} is at most about 1×10⁴ s⁻¹, about 2×10⁴ s⁻¹, about 5×10⁻⁴ s⁻¹, about 1×10⁻³ s⁻¹, about 2×10⁻³ s⁻¹, about 5×10⁻³ s⁻¹, about 1×10⁻² s⁻¹, about 2×10⁻² s⁻¹, about 5×10⁻² s⁻¹, or about 1×10⁻¹ s⁻¹, about 2×10⁻¹ s⁻¹, about 5×10⁻¹ s⁻¹.

### Exemplary Targeting Domains and Exemplary Conjugates

Conjugates herein comprise targeting domains that can be assembled from molecules that engage with a target. In some instances, a targeting domain comprises an antigen-binding region, wherein the antigen binding region engages with a specific target. Such antigen binding regions can comprise CDRs, such as the 3 CDRs generally found in a heavy chain or light chain of an antibody. In some instances, a targeting domain comprises an antigen binding fragment comprising CDRs, such as a VHH (also called single domain antibody (sdAb)). In some instances, a single domain antibody comprises one or more UAAs. In some instances, a targeting domain of a conjugate is selected from a single domain antibody of Table 1, such as any one of C1, C2, C3, C4, C5, C6, C7, C10, C11, C17, C35, or C37. In some instances, a targeting domain of a conjugate is selected from a single domain antibody of Table 1, such as any one of C1, C2, C3, C4, C5, C6, C7, C10, C11, C17, C35, or C37 and one or more UAAs is present in a CDR or in the proximity of a CDR of the single domain antibody. In some instances, the first targeting domain of the conjugate is selected from a single domain antibody of Table 1, such as any one of C1, C2, C3, C4, C5, C6, C7, C10, C11, C17, C35, or C37 and the second targeting domain binds a second target on a second cell. In some instances, the second cell is an immune cell. In some instances, the second targeting domain comprises C6, C7, C37, C38, C39, or C40. In some instances, the second targeting domain is C6 or C7. In some instances, the second targeting domain is C37, C38, C39, or C40. In some instances, the first targeting domain and the second targeting domain are not the same. In some instances, the conjugates comprise a tag, such as a polyhistidine tag, HA tag, or SP tag. In some instances, the conjugates do not comprise the tag. In some cases, the tag, such as a polyhistidine tag, HA tag, or SP tag, may be cleaved.

In some instances, the first targeting domain of the conjugate comprises construct C1 with an unnatural amino acid in CDR1, CDR2, or CDR3. In some instances, the conjugate comprises construct C1 with an unnatural amino acid at any one of positions 26, 28, 29, 30, 99, 102, 103, 105, 108, 110, 111, 112, 113, 114, and 115 in SEQ ID NOs: 1, 4, or 20. In some instances, the conjugate comprises construct C1 with an unnatural amino acid at any one of positions 28, 102, 112, and 113 in SEQ ID NOs: 1, 4, or 20. In some instances, a conjugate comprises a single domain antibody (sdAb), and at least one unnatural amino acid (UAA) within or in the proximity of a CDR region within the sdAb. In some instances, such sdAb comprises SEQ ID NO: 1. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 1. In some instances, a conjugate comprises a first targeting domain having at least 99%, 98%, 97%, 95%, 90%, 85%, 80%, 70%, or at least 65% sequence identity to SEQ ID NO: 1. In some instances, such conjugates have a second targeting domain that binds to an immune cell. In some cases, the second targeting domain comprises C6, C7, C37, C38, C39, or C40, or SEQ ID NOS: 22, 25, 52, 53, 54, or 55. In some cases, the second targeting domain comprises a sequence having at least 99%, 98%, 97%, 95%, 90%, 85%, 80%, 70%, or at least 65% sequence identity to SEQ ID NOS: 22, 25, 52, 53, 54, or 55.

In some instances, the first targeting domain of the conjugate comprises construct C2 with an unnatural amino acid in CDR1, CDR2, or CDR3. In some instances, the conjugate comprises construct C2 with an unnatural amino acid at any one of positions 50, 52, 53, 54, 56, 58, or 100 of SEQ ID NOs: 2, 28, 29, or 30. In some instances, a conjugate comprises a single domain antibody (sdAb), comprising sdAb and at least one unnatural amino acid (UAA) within or in the proximity of a CDR region within the sdAb, wherein the sdAb comprises SEQ ID NO: 2. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 2. In some instances, a conjugate comprises a first targeting domain having at least 99%, 98%, 97%, 95%, 90%, 85%, 80%, 70%, or at least 65% sequence identity to SEQ ID NO: 2. In some instances, such conjugates have a second targeting domain that binds to an immune cell. In some cases, the second targeting domain comprises C6, C7, C37, C38, C39, or C40, or SEQ ID NOS: 22, 25, 52, 53, 54, or 55. In some cases, the second targeting domain comprises a sequence having at least 99%, 98%, 97%, 95%, 90%, 85%, 80%, 70%, or at least 65% sequence identity to SEQ ID NOS: 22, 25, 52, 53, 54, or 55.

In some instances, the first targeting domain of the conjugate comprises construct C3 with an unnatural amino acid in CDR1, CDR2, or CDR3. In some instances, the conjugate comprises construct C3 with an unnatural amino acid at any one of positions 58, 62, 10¹, 10³, or 107 of SEQ ID NO: 3. In some instances, the conjugate comprises an unnatural amino acid in CDR1, CDR2, or CDR3 of C3. In some instances, the conjugate comprises an unnatural amino acid at position 109. In some instances, a conjugate comprises a single domain antibody (sdAb) and at least one unnatural amino acid (UAA) within or in the proximity of a CDR region within the sdAb, wherein the sdAb comprises SEQ ID NO: 3. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 3. In some instances, a conjugate comprises a first targeting domain having at least 99%, 98%, 97%, 95%, 90%, 85%, 80%, 70%, or at least 65% sequence identity to SEQ ID NO: 3. In some instances, such conjugates have a second targeting domain that binds to an immune cell. In some cases, the second targeting domain comprises C6, C7, C37, C38, C39, or C40, or SEQ ID NOS: 22, 25, 52, 53, 54, or 55. In some cases, the second targeting domain comprises a sequence having at least 99%, 98%, 97%, 95%, 90%, 85%, 80%, 70%, or at least 65% sequence identity to SEQ ID NOS: 22, 25, 52, 53, 54, or 55.

In some instances, the first targeting domain of the conjugate comprises construct C4 with an unnatural amino acid in CDR1, CDR2, or CDR3. In some instances, the conjugate comprises construct C3 with an unnatural amino acid at position 109 of SEQ ID NO: 16. In some instances, a conjugate comprises a single domain antibody (sdAb), and at least one unnatural amino acid (UAA) within or in the proximity of a CDR region within the sdAb, wherein the sdAb comprises SEQ ID NO: 16. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 16. In some instances, a conjugate comprises a first targeting domain having at least 99%, 98%, 97%, 95%, 90%, 85%, 80%, 70%, or at least 65% sequence identity to SEQ ID NO: 16. In some instances, such conjugates have a second targeting domain that binds to an immune cell. In some cases, the second targeting domain comprises C6, C7, C37, C38, C39, or C40, or SEQ ID NOS: 22, 25, 52, 53, 54, or 55. In some cases, the second targeting domain comprises a sequence having at least 99%, 98%, 97%, 95%, 90%, 85%, 80%, 70%, or at least 65% sequence identity to SEQ ID NOS: 22, 25, 52, 53, 54, or 55.

In some instances, the first targeting domain of the conjugate comprises construct C5 with an unnatural amino acid in CDR1, CDR2, or CDR3. In some instances, a conjugate comprises a single domain antibody (sdAb), and at least one unnatural amino acid (UAA) within or in the proximity of the a CDR region within the sdAb, wherein the sdAb comprises SEQ ID NO: 18. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 18. In some instances, a conjugate comprises a first targeting domain having at least 99%, 98%, 97%, 95%, 90%, 85%, 80%, 70%, or at least 65% sequence identity to SEQ ID NO: 18. In some instances, such conjugates have a second targeting domain that binds to an immune cell. In some cases, the second targeting domain comprises C6, C7, C37, C38, C39, or C40, or SEQ ID NOS: 22, 25, 52, 53, 54, or 55. In some cases, the second targeting domain comprises a sequence having at least 99%, 98%, 97%, 95%, 90%, 85%, 80%, 70%, or at least 65% sequence identity to SEQ ID NOS: 22, 25, 52, 53, 54, or 55.

In some instances, the second targeting domain of the conjugate comprises construct C6. In some instances, the second targeting domain of the conjugate comprises construct C6 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 22. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 22. In some instances, a conjugate comprises a second targeting domain having at least 99%, 98%, 97%, 95%, 90%, 85%, 80%, 70%, or at least 65% sequence identity to SEQ ID NO: 22. In some instances such conjugates comprise a first targeting domain that binds to a first target on the surface of a tumor cell. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3 rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc 1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1. In some cases, the first targeting domain comprises C1, C2, C3, C4, C5, C10, C11, C17, C35, or C36, or the first targeting domain has at least 99%, 98%, 97%, 95%, 90%, 85%, 80%, 70%, or at least 65% sequence identity to any one of SEQ ID NOs: 1, 2, 3, 16 18, 20, 29, 50, or 51.

In some instances, the second targeting domain of the conjugate comprises construct C7 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 25. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 25. In some instances, a conjugate comprises a first targeting domain having at least 99%, 98%, 97%, 95%, 90%, 85%, 80%, 70%, or at least 65% sequence identity to SEQ ID NO: 25. In some instances such conjugates comprise a first targeting domain that binds to first target on the surface of a tumor cell. In some cases, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, Eph A4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1. In some cases, the first targeting domain comprises C1, C2, C3, C4, C5, C10, C11, C17, C35, or C36, or the first targeting domain has at least 99%, 98%, 97%, 95%, 90%, 85%, 80%, 70%, or at least 65% sequence identity to any one of SEQ ID NOs: 1, 2, 3, 16, 18, 20, 29, 50, or 51.

In some instances, the second targeting domain of the conjugate comprises construct C37. In some instances, the second targeting domain of the conjugate comprises construct C37 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 52. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 52. In some instances, a conjugate comprises a second targeting domain having at least 99%, 98%, 97%, 95%, 90%, 85%, 80%, 70%, or at least 65% sequence identity to SEQ ID NO: 52. In some instances such conjugates comprise a first targeting domain that binds to a first target on the surface of a tumor cell. In some cases, the first target comprises In some cases, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, Eph A3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGaVb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3 rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, Eph A3, Eph A4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MC1R, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1. In some cases, the first targeting domain comprises C1, C2, C3, C4, C5, C10, C11, C17, C35, or C36. In some cases, the first targeting domain has at least 99%, 98%, 97%, 95%, 90%, 85%, 80%, 70%, or at least 65% sequence identity to any one of SEQ ID NOs: 1, 2, 3, 16 18, 20, 29, 50, or 51.

In some instances, the second targeting domain of the conjugate comprises construct C38 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 53. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 53. In some instances, a conjugate comprises a first targeting domain having at least 99%, 98%, 97%, 95%, 90%, 85%, 80%, 70%, or at least 65% sequence identity to SEQ ID NO: 53. In some instances such conjugates comprise a first targeting domain that binds to first target on the surface of a tumor cell. In some cases, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo180, Endoglin, ENPP3, EpCAM, EphA2, EphA3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor 1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MTI-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1. In some cases, the first targeting domain comprises C1, C2, C3, C4, C5, C10, C11, C17, C35, or C36. In some cases, the first targeting domain has at least 99%, 98%, 97%, 95%, 90%, 85%, 80%, 70%, or at least 65% sequence identity to any one of SEQ ID NOs: 1, 2, 3, 16, 18, 20, 29, 50, or 51.

In some instances, the conjugate comprises construct C39 with an unnatural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 54. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 54. In some instances, a conjugate comprises a first targeting domain having at least 99%, 98%, 97%, 95%, 90%, 85%, 80%, 70%, or at least 65% sequence identity to SEQ ID NO: 54. In some instances such conjugates comprise a first targeting domain that binds to first target on the surface of a tumor cell. In some cases, the first target comprises In some cases, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, Eph A2, EphA3, Eph A4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1. In some cases, the first targeting domain comprises C1, C2, C3, C4, C5, C10, C11, C17, C35, or C36. In some cases, the first targeting domain has at least 99%, 98%, 97%, 95%, 90%, 85%, 80%, 70%, or at least 65% sequence identity to any one of SEQ ID NOs: 1, 2, 3, 16, 18, 20, 29, 50, or 51.

In some instances, the conjugate comprises construct C40 with an un natural amino acid in CDR1, CDR2, or CDR3. In some instances, a conjugate comprises a single domain antibody (sdAb), and at least one unnatural amino acid (UAA) within or in the proximity of a CDR region within the sdAb, wherein the sdAb comprises SEQ ID NO: 55. In some instances, a conjugate comprises a single domain antibody (sdAb), and at least one unnatural amino acid (UAA) within or in the proximity of a CDR region within the sdAb, wherein the sdAb comprises a sequence having at least 99%, 98%, 97%, 95%, 90%, 85%, 80%, 70%, or at least 65% sequence identity to SEQ ID NO: 55. In some instances, a conjugate comprises a first targeting domain having at least 99%, 98%, 97%, 95%, 90%, 85%, 80%, 70%, or at least 65% sequence identity to SEQ ID NO: 55. In some instances such conjugates comprise a first targeting domain that binds to first target on the surface of a tumor cell. In some cases, the first target comprises In some cases, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, Eph A2, Eph A3, Eph A4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1.

In some cases, the first targeting domain comprises C1, C2, C3, C4, C5, C10, C11, C17, C35, or C36. In some cases, the first targeting domain has at least 99%, 98%, 97%, 95%, 90%, 85%, 80%, 70%, or at least 65% sequence identity to any one of SEQ ID NOs: 1, 2, 3, 16, 18, 20, 29, 50, or 51.

Conjugates described herein may comprise two or more targeting domains. In some instances, a conjugate with a first targeting domain and a second targeting domain comprises any one of SEQ ID NOs: 19, 21, 23, 24, 26, 27, 30, 35-49, 57, or 64. In some instances, the conjugate has at least 99%, 98%, 97%, 95%, 90%, 85%, 80%, 70%, or at least 65% sequence identity to any one of SEQ ID NOs: 19, 21, 23, 24, 26, 27, 30, 35-49, 57, or 64. In some instances, first and second targeting domains are attached to each other via a linker. In some instances, the conjugate is a fusion protein. In some instances, the linker comprises linker L1 (SEQ ID NO: 14), L2 (SEQ ID NO: 31), L3 (SEQ ID NO: 32), L4 (SEQ ID NO: 33), OR L5 (SEQ ID NO: 34). In some instances, a conjugate comprises any one of SEQ ID NOs: 19, 21, 23, 24, 26, 27, 28, 30, 35-49, 57, or 64. In some instances, a conjugate has at least 99%, 98%, 97%, 95%, 90%, 85%, 80%, 70%, or at least 65% sequence identity to any one of SEQ ID NOs: 19, 21, 23, 24, 26, 27, 28, 30, 35-49, 57, or 64. In some instances, the conjugate comprises any one of SEQ ID NOS: 19, 21, 23, 24, 26, 27, 28, 30, 35-49, 57, or 64 without a terminal his6, HA, or SP tag.

Exemplary targeting domain-related sequences are described herein in Table 1.

TABLE 1
Exemplary Targeting Domain-Related Sequences

| SEQ | | |
|---|---|---|
| ID | | |
| NO | Description | Sequence |
| 1 | C1 | QVQLQESGGGSVQAGGSLRLSCTAPGYTDSNYYMSWFRQAP |
| | | GKEREWVAGVNTGRGSTSYADSVKGRFTISQDNAKNTMFLQ |
| | | MNSLKPEDTAIYYCAVAACHFCDSLPKTQDEYILWGQGTQV |
| | | TVSSAAAYPYDVPDYGS |
| 2 | C2 | EVQLVESGGGLVQPGGSLTLSCAASRFMISEYSMHWVRQAP |
| | | GKGLEWVSTINPAGTTDYAESVKGRFTISRDNAKNTLYLQM |
| | | NSLKPEDTAVYYCDGYGYRGQGTQVTVSS |
| 3 | C3 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRDYSLSSSSDDFDYWGQGTQVTVSS |
| | | AAAYPYDVPDYGS |
| 4 | C10-102FSY | QVQLQESGGGSVQAGGSLRLSCTAPGYTDSNYYMSWFRQAP |
| | | GKEREWVAGVNTGRGSTSYADSVKGRFTISQDNAKNTMFLQ |
| | | MNSLKPEDTAIYYCAVAAAXFADSLPKTQDEYILWGQGTQV |
| | | TVSSAAAYPYDVPDYGS |
| 5 | C1_CDR1_26- | GYTDSNYYMS |
| | 35 | |
| 6 | C1_CDR2_50- | VNTGRGSTSYADSVKG |
| | 66 | |
| 7 | C1_CDR3_99- | AACHFCDSLPKTQDEYIL |
| | 116 | |
| 8 | C2_CDR1_26- | RFMISEYSMH |
| | 35 | |
| 9 | C2_CDR2_50- | TINPAGTTDYAESVKG |
| | 65 | |
| 10 | C2_CDR3_96- | DGYGY |
| | 100 | |
| 11 | C3_CDR1_26- | GWPYSTYSMN |
| | 35 | |
| 12 | C3_CDR2_50- | GISSTMSGIIFAESKAG |
| | 65 | |
| 13 | C3_CDR3_99- | RRDYSLSSSSDDFDY |
| | 113 | |
| 14 | L1 | GGGGSGGGGS |
| 15 | PelB | MKYLLPTAAAGLLLLAAQPAMA |
| 16 | C4 | MGQVKLEESGGGSVQTGGSLRLTCAASGRTSRSYGMGWFR |
| | | QAPGKEREFVSGISWRGDSTGYADSVKGRFTISRDNAKNTVD |
| | | LQMNSLKPEDTAIYYCAAAAGSAWYGTLYEYDYWGQGTQV |
| | | TVSS |
| 17 | C4-109FSY | QVKLEESGGGSVQTGGSLRLTCAASGRTSRSYGMGWFRQAP |
| | | GKEREFVSGISWRGDSTGYADSVKGRFTISRDNAKNTVDLQ |
| | | MNSLKPEDTAIYYCAAAAGSAWYGTLXEYDYWGQGTQVTV |
| | | SS |
| 18 | C5 | QVQLQESGGGLVQPGGSLRLSCAASGRTFSSYAMGWFRQAP |
| | | GKQREFVAAIRWSGGYTYYTDSVKGRFTISRDNAKTTVYLQ |
| | | MNSLKPEDTAVYYCAATYLSSDYSRYALPQRPLDYDYWGQ |
| | | GTQVTVSS |
| 19 | C9 | QVKLEESGGGSVQTGGSLRLTCAASGRTSRSYGMGWFRQAP |
| | | GKEREFVSGISWRGDSTGYADSVKGRFTISRDNAKNTVDLQ |
| | | MNSLKPEDTAIYYCAAAAGSAWYGTLXEYDYWGQGTQVTV |
| | | SSGGGGSGGGGSQVQLQESGGGLVQPGGSLRLSCAASGRTFS |
| | | SYAMGWFRQAPGKQREFVAAIRWSGGYTYYTDSVKGRFTIS |
| | | RDNAKTTVYLQMNSLKPEDTAVYYCAATYLSSDYSRYALPQ |
| | | RPLDYDYWGQGTQVTVSS |
| 20 | C10 | QVQLQESGGGSVQAGGSLRLSCTAPGYTDSNYYMSWFRQAP |
| | | GKEREWVAGVNTGRGSTSYADSVKGRFTISQDNAKNTMFLQ |
| | | MNSLKPEDTAIYYCAVAAAHFADSLPKTQDEYILWGQGTQV |
| | | TVSSAAAYPYDVPDYGS |
| 21 | C11-102FSY | QVQLQESGGGSVQAGGSLRLSCTAPGYTDSNYYMSWFRQAP |
| | | GKEREWVAGVNTGRGSTSYADSVKGRFTISQDNAKNTMFLQ |
| | | MNSLKPEDTAIYYCAVAAAXFADSLPKTQDEYILWGQGTQV |
| | | TVSSAAAYPYDVPDYGSGGGGSGGGGSQVQLQESGGGSVQ |
| | | AGGSLRLSCTAPGYTDSNYYMSWFRQAPGKEREWVAGVNT |
| | | GRGSTSYADSVKGRFTISQDNAKNTMFLQMNSLKPEDTAIYY |
| | | CAVAAAHFADSLPKTQDEYILWGQGTQVTVSSAAAYPYDVP |
| | | DYGS |
| 22 | C6 | EVQLVESGGGLVQPGGSLKLSCAASGFTFNKYAMNWVRQA |
| | | PGKGLEWVARIRSKYNNYATYYADSVKDRFTISRDDSKNTA |
| | | YLQMNNLKTEDTAVYYCVRHGNFGNSYISYWAYWGQGTL |
| | | VTVSS |
| 23 | C12 | MGQVKLEESGGGSVQTGGSLRLTCAASGRTSRSYGMGWFR |
| | | QAPGKEREFVSGISWRGDSTGYADSVKGRFTISRDNAKNTVD |
| | | LQMNSLKPEDTAIYYCAAAAGSAWYGTLYEYDYWGQGTQV |
| | | TVSSGGGGSGGGSEVQLVESGGGLVQPGGSLKLSCAASGFTF |
| | | NKYAMNWVRQAPGKGLEWVARIRSKYNNYATYYADSVKD |
| | | RFTISRDDSKNTAYLQMNNLKTEDTAVYYCVRHGNFGNSYI |
| | | SYWAYWGQGTLVTVSS |
| 24 | C13 | MGQVKLEESGGGSVQTGGSLRLTCAASGRTSRSYGMGWFR |
| | | QAPGKEREFVSGISWRGDSTGYADSVKGRFTISRDNAKNTVD |
| | | LQMNSLKPEDTAIYYCAAAAGSAWYGTLXEYDYWGQGTQV |
| | | TVSSGGGGSGGGSEVQLVESGGGLVQPGGSLKLSCAASGFTF |
| | | NKYAMNWVRQAPGKGLEWVARIRSKYNNYATYYADSVKD |
| | | RFTISRDDSKNTAYLQMNNLKTEDTAVYYCVRHGNFGNSYI |
| | | SYWAYWGQGTLVTVSS |
| 25 | C7 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSS |
| 26 | C14 | MGQVKLEESGGGSVQTGGSLRLTCAASGRTSRSYGMGWFR |
| | | QAPGKEREFVSGISWRGDSTGYADSVKGRFTISRDNAKNTVD |
| | | LQMNSLKPEDTAIYYCAAAAGSAWYGTLYEYDYWGQGTQV |
| | | TVSSGGGGSGGGGSGGGGSEVQLVESGGGLVQPGESLTLSC |
| | | VVAGSIFSFAMSWYRQAPGKERELVARIGSDDRVTYADSVK |
| | | GRFTISRDNIKRTAGLQMNSLKPEDTAVYYCNAQTDLRDWT |
| | | VREYWGQGTQVTVSS |
| 27 | C15 | MGQVKLEESGGGSVQTGGSLRLTCAASGRTSRSYGMGWFR |
| | | QAPGKEREFVSGISWRGDSTGYADSVKGRFTISRDNAKNTVD |
| | | LQMNSLKPEDTAIYYCAAAAGSAWYGTLXEYDYWGQGTQV |
| | | TVSSGGGGSGGGGSGGGGSEVQLVESGGGLVQPGESLTLSC |
| | | VVAGSIFSFAMSWYRQAPGKERELVARIGSDDRVTYADSVK |
| | | GRFTISRDNIKRTAGLQMNSLKPEDTAVYYCNAQTDLRDWT |
| | | VREYWGQGTQVTVSS |
| 28 | C16 | EVQLVESGGGLVQPGGSLTLSCAASRFMISEYSMHWVRQAP |
| | | GKGLEWVSTINPAGTTDYAESVKGRFTISRDNAKNTLYLQM |
| | | NSLKPEDTAVYYCDGYGYRGQGTQVTVSSGGGGSGGGSEV |
| | | QLVESGGGLVQPGGSLKLSCAASGFTFNKYAMNWVRQAPG |
| | | KGLEWVARIRSKYNNYATYYADSVKDRFTISRDDSKNTAYL |
| | | QMNNLKTEDTAVYYCVRHGNFGNSYISYWAYWGQGTLVT |
| | | VSS |
| 29 | C17 | EVQLVESGGGLVQPGGSLTLSCAASRFMISEYSMHWVRQAP |
| | | GKGLEWVSTINPXGTTDYAESVKGRFTISRDNAKNTLYLQM |
| | | NSLKPEDTAVYYCDGYGYRGQGTQVTVSS |
| 30 | C18 | EVQLVESGGGLVQPGGSLTLSCAASRFMISEYSMHWVRQAP |
| | | GKGLEWVSTINPXGTTDYAESVKGRFTISRDNAKNTLYLQM |
| | | NSLKPEDTAVYYCDGYGYRGQGTQVTVSSGGGGSGGGSEV |
| | | QLVESGGGLVQPGGSLKLSCAASGFTFNKYAMNWVRQAPG |
| | | KGLEWVARIRSKYNNYATYYADSVKDRFTISRDDSKNTAYL |
| | | QMNNLKTEDTAVYYCVRHGNFGNSYISYWAYWGQGTLVT |
| | | VSS |
| 31 | L2 | GGGGSGGGGSGGGGS |
| 32 | L3 | GGGGSGGGGSGGGGSGGGGS |
| 33 | L4 | GGGGSGGGGSGGGGSGGGGSGGGGS |
| 34 | L5 | SPSTPPTPSPSTPP |
| 35 | C20 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSGGGGS |
| | | GGGGSGGGGSQVQLQESGGGSVEAGGSLRLSCARSGWPYST |
| | | YSMNWFRQAPGKEREAVAGISSTMSGIIFAESKAGQFTISQD |
| | | NAKNTVYLQMNNLKPEDTAIYYCAARRXFSLSSSSDDFDYW |
| | | GQGTQVTVSSAAAYPYDVPDYGSSPSTPPTPSPSTPPHHHHH |
| | | H |
| 36 | C21 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSGGGGS |
| | | GGGGSQVQLQESGGGSVEAGGSLRLSCARSGWPYSTYSMN |
| | | WFRQAPGKEREAVAGISSTMSGIIFAESKAGQFTISQDNAKNT |
| | | VYLQMNNLKPEDTAIYYCAARRXFSLSSSSDDFDYWGQGTQ |
| | | VTVSSAAAYPYDVPDYGSSPSTPPTPSPSTPPHHHHHH |
| 37 | C22 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSGGGGS |
| | | GGGGSGGGGSGGGGSQVQLQESGGGSVEAGGSLRLSCARSG |
| | | WPYSTYSMNWFRQAPGKEREAVAGISSTMSGIIFAESKAGQF |
| | | TISQDNAKNTVYLQMNNLKPEDTAIYYCAARRXFSLSSSSDD |
| | | FDYWGQGTQVTVSSAAAYPYDVPDYGSSPSTPPTPSPSTPPH |
| | | HHHHH |
| 38 | C23 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSGGGGS |
| | | GGGGSGGGGSGGGGSGGGGSQVQLQESGGGSVEAGGSLRLS |
| | | CARSGWPYSTYSMNWFRQAPGKEREAVAGISSTMSGIIFAES |
| | | KAGQFTISQDNAKNTVYLQMNNLKPEDTAIYYCAARRXFSL |
| | | SSSSDDFDYWGQGTQVTVSSAAAYPYDVPDYGSSPSTPPTPS |
| | | PSTPPHHHHHH |
| 39 | C24 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSSPSTPP |
| | | TPSPSTPPQVQLQESGGGSVEAGGSLRLSCARSGWPYSTYSM |
| | | NWFRQAPGKEREAVAGISSTMSGIIFAESKAGQFTISQDNAK |
| | | NTVYLQMNNLKPEDTAIYYCAARRXFSLSSSSDDFDYWGQG |
| | | TQVTVSSAAAYPYDVPDYGSSPSTPPTPSPSTPPHHHHHH |
| 40 | C25 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSSGGGGS |
| | | GGGGSGGGGSGGGGSQVQLQESGGGSVEAGGSLRLSCARSG |
| | | WPYSTYSMNWFRQAPGKEREAVAGISSTMSGIIFAESKAGQF |
| | | TISQDNAKNTVYLQMNNLKPEDTAIYYCAARRYFSLSSSSDD |
| | | FDYWGQGTQVTVSSAAAYPYDVPDYGSSPSTPPTPSPSTPPH |
| | | HHHHH |
| 41 | C26 | EVQLVESGGGPVQAGGSLRLSCAASGRTYRGYSMGWFRQA |
| | | PGKEREFVAAIVWSGGNTYYEDSVKGRFTISRDNAKNTMYL |
| | | QMTSLKPEDSATYYCAAKIRPYIFKIAGQYDYWGQGTLVTVS |
| | | SGGGGSGGGGSGGGGSGGGGSQVQLQESGGGSVEAGGSLRL |
| | | SCARSGWPYSTYSMNWFRQAPGKEREAVAGISSTMSGIIFAE |
| | | SKAGQFTISQDNAKNTVYLQMNNLKPEDTAIYYCAARRYFS |
| | | LSSSSDDFDYWGQGTQVTVSSAAAYPYDVPDYGSSPSTPPTP |
| | | SPSTPPHHHHHH |
| 42 | C27 | EVQLVESGGGPVQAGGSLRLSCAASGRTYRGYSMGWFRQA |
| | | PGKEREFVAAIVWSGGNTYYEDSVKGRFTISRDNAKNTMYL |
| | | QMTSLKPEDSATYYCAAKIRPYIFKIAGQYDYWGQGTLVTVS |
| | | SGGGGSGGGGSGGGGSGGGGSQVQLQESGGGSVEAGGSLRL |
| | | SCARSGWPYSTYSMNWFRQAPGKEREAVAGISSTMSGIIFAE |
| | | SKAGQFTISQDNAKNTVYLQMNNLKPEDTAIYYCAARRXFS |
| | | LSSSSDDFDYWGQGTQVTVSSAAAYPYDVPDYGSSPSTPPTP |
| | | SPSTPPHHHHHH |
| 43 | C28 | EVQLVESGGGLVQPGGSLRLSCAASGDIYKSFDMGWYRQAP |
| | | GKQRDLVAVIGSRGNNRGRTNYADSVKGRFTISRDGTGNTV |
| | | YLLMNKLRPEDTAIYYCNTAPLVAGRPWGRGTLVTVSSGGG |
| | | GSGGGGSGGGGSGGGGSQVQLQESGGGSVEAGGSLRLSCAR |
| | | SGWPYSTYSMNWFRQAPGKEREAVAGISSTMSGIIFAESKAG |
| | | QFTISQDNAKNTVYLQMNNLKPEDTAIYYCAARRYFSLSSSS |
| | | DDFDYWGQGTQVTVSSAAAYPYDVPDYGSSPSTPPTPSPSTP |
| | | PHHHHHH |
| 44 | C29 | EVQLVESGGGLVQPGGSLRLSCVASGDVHKINFLGWYRQAP |
| | | GKEREKVAHISIGDQTDYADSAKGRFTISRDESKNMVYLQM |
| | | NSLKPEDTAVYFCRAFSRIYPYDYWGQGTLVTVSSGGGGSG |
| | | GGGSGGGGSGGGGSQVQLQESGGGSVEAGGSLRLSCARSG |
| | | WPYSTYSMNWFRQAPGKEREAVAGISSTMSGIIFAESKAGQF |
| | | TISQDNAKNTVYLQMNNLKPEDTAIYYCAARRYFSLSSSSDD |
| | | FDYWGQGTQVTVSSAAAYPYDVPDYGSSPSTPPTPSPSTPPH |
| | | HHHHH |
| 45 | C30 | EVQLVESGGGLVQPGGSLRLSCVASGDVHKINFLGWYRQAP |
| | | GKEREKVAHISIGDQTDYADSAKGRFTISRDESKNMVYLQM |
| | | NSLKPEDTAVYFCRAFSRIYPYDYWGQGTLVTVSSGGGGSG |
| | | GGGSGGGGSGGGGSQVQLQESGGGSVEAGGSLRLSCARSG |
| | | WPYSTYSMNWFRQAPGKEREAVAGISSTMSGIIFAESKAGQF |
| | | TISQDNAKNTVYLQMNNLKPEDTAIYYCAARRXFSLSSSSDD |
| | | FDYWGQGTQVTVSSAAAYPYDVPDYGSSPSTPPTPSPSTPPH |
| | | HHHHH |
| 46 | C31 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRYFSLSSSSDDFDYWGQGTQVTVSSG |
| | | GGGSGGGGSGGGGSGGGGSEVQLVESGGGPVQAGGSLRLSC |
| | | AASGRTYRGYSMGWFRQAPGKEREFVAAIVWSGGNTYYED |
| | | SVKGRFTISRDNAKNTMYLQMTSLKPEDSATYYCAAKIRPYI |
| | | FKIAGQYDYWGQGTLVTVSSAAAYPYDVPDYGSSPSTPPTPS |
| | | PSTPPHHHHHH |
| 47 | C32 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRYFSLSSSSDDFDYWGQGTQVTVSSG |
| | | GGGSGGGGSGGGGSGGGGSEVQLVESGGGLVQPGGSLRLSC |
| | | AASGDIYKSFDMGWYRQAPGKQRDLVAVIGSRGNNRGRTN |
| | | YADSVKGRFTISRDGTGNTVYLLMNKLRPEDTAIYYCNTAPL |
| | | VAGRPWGRGTLVTVSSAAAYPYDVPDYGSSPSTPPTPSPSTPP |
| | | HHHHHH |
| 48 | C33 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRYFSLSSSSDDFDYWGQGTQVTVSSG |
| | | GGGSGGGGSGGGGSGGGGSEVQLVESGGGLVQPGGSLRLSC |
| | | VASGDVHKINFLGWYRQAPGKEREKVAHISIGDQTDYADSA |
| | | KGRFTISRDESKNMVYLQMNSLKPEDTAVYFCRAFSRIYPYD |
| | | YWGQGTLVTVSSAAAYPYDVPDYGSSPSTPPTPSPSTPPHHH |
| | | HHH |
| 49 | C34 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRXFSLSSSSDDFDYWGQGTQVTVSSG |
| | | GGGSGGGGSGGGGSGGGGSEVQLVESGGGLVQPGGSLRLSC |
| | | VASGDVHKINFLGWYRQAPGKEREKVAHISIGDQTDYADSA |
| | | KGRFTISRDESKNMVYLQMNSLKPEDTAVYFCRAFSRIYPYD |
| | | YWGQGTLVTVSSAAAYPYDVPDYGSSPSTPPTPSPSTPPHHH |
| | | HHH |
| 50 | C35 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRXFSLSSSSDDFDYWGQGTQVTVSSA |
| | | AAYPYDVPDYGS |
| 51 | C36 | QVQLQESGGGSVEAGGSLRLSCARSGWPYSTYSMNWFRQAP |
| | | GKEREAVAGISSTMSGIIFAESKAGQFTISQDNAKNTVYLQM |
| | | NNLKPEDTAIYYCAARRYFSLSSSSDDFDYWGQGTQVTVSSA |
| | | AAYPYDVPDYGSSPSTPPTPSPSTPPHHHHHH |
| 52 | C37 | EVQLVESGGGLVQPGESLTLSCVVAGSIFSFAMSWYRQAPGK |
| | | ERELVARIGSDDRVTYADSVKGRFTISRDNIKRTAGLQMNSL |
| | | KPEDTAVYYCNAQTDLRDWTVREYWGQGTQVTVSS |
| 53 | C38 | EVQLVESGGGPVQAGGSLRLSCAASGRTYRGYSMGWFRQA |
| | | PGKEREFVAAIVWSGGNTYYEDSVKGRFTISRDNAKNTMYL |
| | | QMTSLKPEDSATYYCAAKIRPYIFKIAGQYDYWGQGTLVTVS |
| | | S |
| 54 | C39 | EVQLVESGGGLVQPGGSLRLSCAASGDIYKSFDMGWYRQAP |
| | | GKQRDLVAVIGSRGNNRGRTNYADSVKGRFTISRDGTGNTV |
| | | YLLMNKLRPEDTAIYYCNTAPLVAGRPWGRGTLVTVSS |
| 55 | C40 | EVQLVESGGGLVQPGGSLRLSCVASGDVHKINFLGWYRQAP |
| | | GKEREKVAHISIGDQTDYADSAKGRFTISRDESKNMVYLQM |
| | | NSLKPEDTAVYFCRAFSRIYPYDYWGQGTLVTVSS |
| 56 | L6 | GGGGSGGGS |
| 57 | C16 | EVQLVESGGGLVQPGGSLTLSCAASRFMISEYSMHWVRQAP |
| | | GKGLEWVSTINPAGTTDYAESVKGRFTISRDNAKNTLYLQM |
| | | NSLKPEDTAVYYCDGYGYRGQGTQVTVSSGGGGSGGGSEV |
| | | QLVESGGGLVQPGGSLKLSCAASGFTFNKYAMNWVRQAPG |
| | | KGLEWVARIRSKYNNYATYYADSVKDRFTISRDDSKNTAYL |
| | | QMNNLKTEDTAVYYCVRHGNFGNSYISYWAYWGQGTLVT |
| | | VSSHHHHHH |
| 64 | C41 | EVQLVESGGGLVQPGGSLRLSCAASGDIYKSFDMGWYRQAP |
| | | GKQRDLVAVIGSRGNNRGRTNYADSVKGRFTISRDGTGNTV |
| | | YLLMNKLRPEDTAIYYCNTAPLVAGRPWGRGTLVTVSSGGG |
| | | GSGGGGSGGGGSGGGGSQVQLQESGGGSVEAGGSLRLSCAR |
| | | SGWPYSTYSMNWFRQAPGKEREAVAGISSTMSGIIFAESKAG |
| | | QFTISQDNAKNTVYLQMNNLKPEDTAIYYCAARRXFSLSSSS |
| | | DDFDYWGQGTQVTVSSAAAYPYDVPDYGSSPSTPPTPSPSTP |
| | | PHHHHHH |
| 65 | L7 | GGGGS |

NOTE:
X indicates the position of the FSY incorporation site. Sequences in the table with a tag, such as a His6 purification tag, an HA tag, a sortase recognition site, an SP linker (L5) and/or PelB leader sequence, are also embodied herein. Sequences of Table 1 may be present without a tag.

Unnatural amino acids (UAAs) may be incorporated into conjugates described herein. In some instances, UAAs are present in a targeting domain of the conjugate. In some instances, UAA are configured within a targeting domain to covalently bind to a target. In some instances, a conjugate comprises one, two, three, four, or more UAAs. In some instances, a conjugate comprises a first targeting domain comprising a first UAA and second targeting domain comprising a second UAA. In some instances, a UAA is configured within a targeting domain to covalently bind an amino acid present in the target when the targeting domain engages the target. In some instances, such UAAs comprise nucleophilic amino acids. In some instances, UAAs form covalent bonds with lysine, histidine, or tyrosine. In some instances, UAAs are located in a target-binding domain. In some instances, one or more UAAs is located in one or more CDRs of a targeting domain, such as a targeting domain comprising an antibody or antigen binding fragment, for example, a single domain antibody. In some embodiments, the UAA comprises an aryl-fluoro sulfate moiety. In some instances, the UAA is genetically encoded into the conjugates described herein. In some instances, the UAA comprises a variant of tyrosine or lysine. In some embodiments, the unnatural amino acid comprises a structure of Formula I:

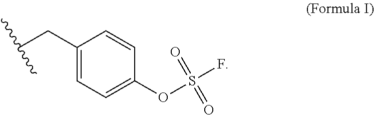 [SMILES: *Cc1ccc(OS(=O)(=O)F)cc1]

In some embodiments, the unnatural amino acid comprises a structure of Formula II:

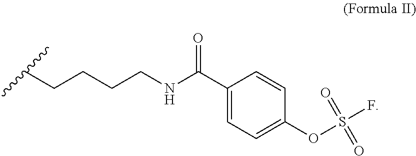 [SMILES: *CCCCNC(=O)c1ccc(OS(=O)(=O)F)cc1]

In some embodiments, the unnatural amino acid is 2-amino-3-(4-((fluorosulfonyl)oxy)phenyl) propanoic acid:

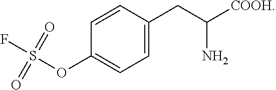 [SMILES: NC(Cc1ccc(OS(=O)(=O)F)cc1)C(=O)O]

In some embodiments, the unnatural amino acid is fluorosulfonyltyrosine (FSY):

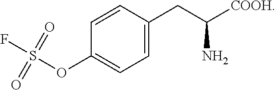 [SMILES: N[C@@H](Cc1ccc(OS(=O)(=O)F)cc1)C(=O)O]

In some embodiments, the unnatural amino acid is N6-(4-((fluorosulfonyl)oxy)benzoyl)lysine:

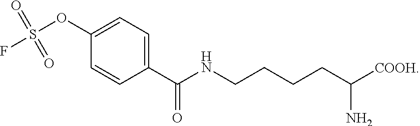 [SMILES: NC(CCCCNC(=O)c1ccc(OS(=O)(=O)F)cc1)C(=O)O]

In some embodiments, the unnatural amino acid is fluorosulfonyloxybenzoyl-L-lysine (FSK):

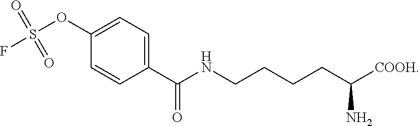 [SMILES: N[C@@H](CCCCNC(=O)c1ccc(OS(=O)(=O)F)cc1)C(=O)O]

In some embodiments, the unnatural amino acid is 2-amino-3-(3-fluoro-4-((fluorosulfonyl)oxy)phenyl) propanoic acid:

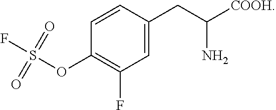 [SMILES: NC(Cc1ccc(OS(=O)(=O)F)c(F)c1)C(=O)O]

In some embodiments, the unnatural amino acid is fluoro-fluorosulfonyltyrosine (FFY):

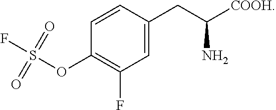 [SMILES: N[C@@H](Cc1ccc(OS(=O)(=O)F)c(F)c1)C(=O)O]

In some embodiments, the unnatural amino acid (UAA) has a structure of Formula (IA):

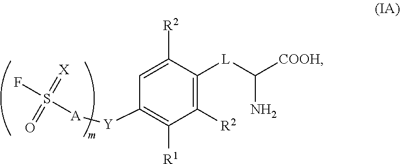

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, m is 1; when Y is —N═, m is 2.

In some embodiments, the UAA of Formula (IA) has a structure of Formula (IA-a):

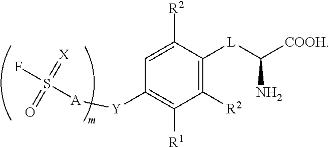

In some embodiments, the UAA of Formula (IA) has a structure of Formula (IA-b):

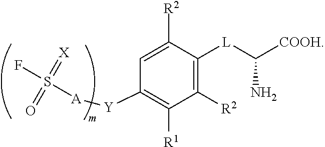

In some embodiments, the UAA of Formula (IA) has a structure of Formula (IB):

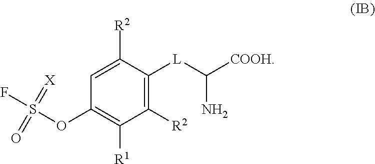

In some embodiments, the UAA of Formula (IB) has a structure of Formula (IB-a):

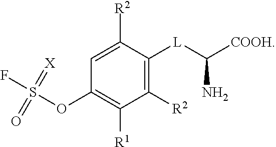

In some embodiments, the UAA of Formula (IB) has a structure of Formula (IB-b):

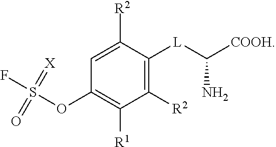

In some embodiments, the UAA of Formula (IA) has a structure of Formula (IC):

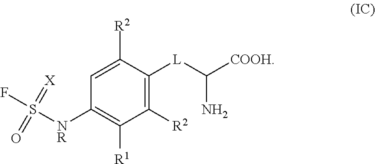

In some embodiments, the UAA of Formula (IC) has a structure of Formula (IC-a):

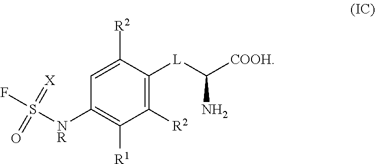

In some embodiments, the UAA of Formula (IB) has a structure of Formula (IB-b):

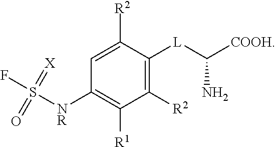

In some embodiments, R is hydrogen.

In some embodiments, the UAA of Formula (IA) has a structure of Formula (ID):

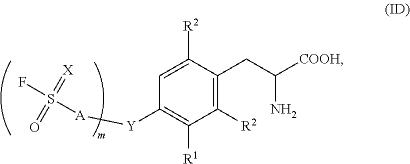

wherein each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, mis 1; when Y is —N═, mis 2.

In some embodiments, the UAA of Formula (ID) has a structure of Formula (ID-a):

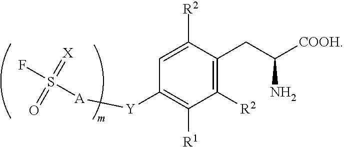

In some embodiments, the UAA of Formula (ID) has a structure of Formula (ID-b):

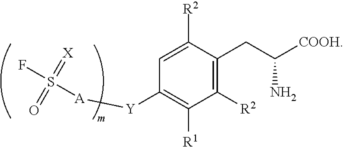

In some embodiments, the UAA of Formula (IA) has a structure of Formula (IE):

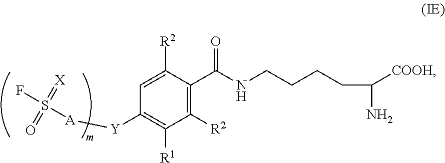

wherein each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

In some embodiments, the UAA of Formula (IE) has a structure of Formula (IE-a):

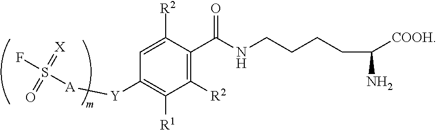

In some embodiments, the UAA of Formula (IE) has a structure of Formula (IE-b):

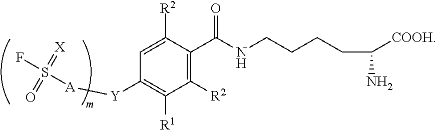

In certain embodiments, Y is a bond, —O— or —NR—, mis 1. In other embodiments, Y is —N═, m is 2. In certain embodiments, Y is —O— and m is 1. In other embodiments, Y is —NR— and m is 1. In other embodiments, Y is a bond, and m is 1. In other embodiments, Y is O— or —NR—, m is 1.

In some embodiments, the UAA of Formula (IA) has a structure of Formula (IIA):

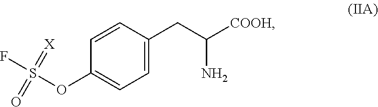

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

In some embodiments, the UAA of Formula (IIA) has a structure of Formula (IIA-a):

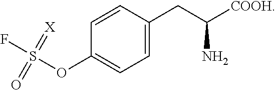

In some embodiments, the UAA of Formula (IIA) has a structure of Formula (IIA-b):

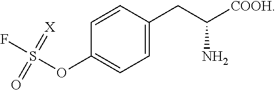

In some embodiments, the UAA of Formula (IA) has a structure of Formula (IIB):

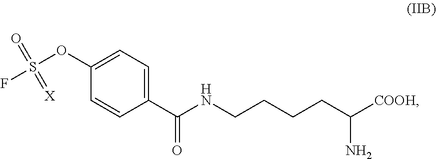

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

In some embodiments, the UAA of Formula (IIB) has a structure of Formula (IIB-a):

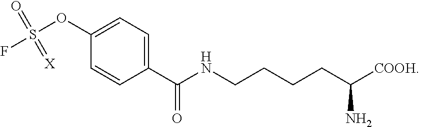

In some embodiments, the UAA of Formula (IIB) has a structure of Formula (IIB-b):

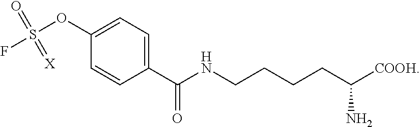

In some embodiments, the unnatural amino acid (UAA) has a structure of

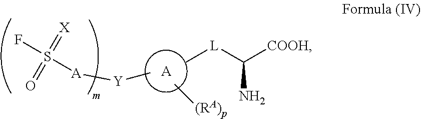

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, mis 2.

In some embodiments, A is a bond. In other embodiments, A is —(CH₂)ₙ—. In some embodiments, n is 1, 2, 3 or 4. In one embodiment, n is 1. In another embodiment, n is 2. In yet another embodiment, n is 3. In yet another embodiment, n is 4.

In some embodiments, Ring A is a 5-membered ring. In some embodiments, Ring A is a 6-membered ring. In some embodiments, Ring A is aryl. In some embodiments, Ring A is heteroaryl. In some embodiments, Ring A is 6-membered aryl. In some embodiments, Ring A is 6-membered heteroaryl. In one embodiment, Ring A is phenyl.

In some embodiments, p is 0. In some embodiments, p is an integer of 1 to 4. In some embodiments, p is an integer of 1 to 3. In one embodiment, p is 1. In another embodiment, p is 2. In yet another embodiment, p is 3. In yet another embodiment, p is 4.

In some embodiments, each R^{A} is independently —OH, halo or optionally substituted alkyl. In some embodiments, each R^{A} is independently halo or optionally substituted alkyl. In some embodiments, each R^{A} is independently —OH or optionally substituted alkyl. In some embodiments, each R^{A} is independently optionally substituted alkyl. In some embodiments, each R^{A} is independently substituted alkyl. In some embodiments, each R^{A} is independently unsubstituted alkyl. In one embodiment, each R^{A} is iodo. In another embodiment, each R^{A} is methyl. In one specific embodiment, wherein p is 1, R^{A} is iodo. In another specific embodiment, wherein p is 2, each R^{A} is methyl.

In some embodiments, Y is a bond, —O—, —NR—, or —N═. In some embodiments, Y is —O—, —NR—, or —N═. In some embodiments, Y is a bond, —O—, or —NR—. In some embodiments, Y is a bond, —O—, or —N═. In some embodiments, Y is a bond, —NR—, or —N═. In some embodiments, Y is-O— or —NR—. In some embodiments, Y is —O— or —N═. In some embodiments, Y is —NR— or —N═. In one embodiment, Y is a bond. In another embodiment, Y is —O—. In yet another embodiment, Y is —NR—. In yet another embodiment, Y is —N═.

In some embodiments, L is —(CH₂)ₚ—. In other embodiments, L is —C(O)NH—(CH₂)ₚ—. In some embodiments, p is an integer of 1 to 6, 1 to 5, 1 to 4, 1 to 3, or 1 to 2. In one embodiment, p is 1 to 4. In another embodiment, p is 1 or 2. In yet another embodiment p is 1 or 4. In one embodiment, L is —CH₂—. In another embodiment, L is —C(O)NH—(CH₂)₄—.

In some embodiments, R is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, R is hydrogen or substituted or unsubstituted alkyl. In one embodiment, R is hydrogen. In another embodiment, R is substituted or unsubstituted C₁₋₆ alkyl. In yet another embodiment, R is unsubstituted C₁₋₆ alkyl. In one embodiment R is methyl. In another embodiment, R is hydrogen or methyl.

In some embodiments, R′ is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl. In some embodiments, R′ is hydrogen, substituted or unsubstituted alkyl, substituted or unsubstituted heteroalkyl, substituted or unsubstituted aryl, or substituted or unsubstituted heteroaryl. In some embodiments, R′ is hydrogen, substituted or unsubstituted alkyl, substituted or unsubstituted aryl, substituted or unsubstituted heteroaryl, or substituted or unsubstituted heterocycloalkyl. In some embodiments, R′ is hydrogen, substituted or unsubstituted alkyl, or substituted or unsubstituted aryl. In embodiment, R′ is hydrogen. In another embodiment, R′ is substituted or unsubstituted. In yet another embodiment, R′ is substituted or unsubstituted aryl.

In some embodiments, R¹ is hydrogen. In some embodiments, R¹ is fluoro. In some embodiments, R¹ is iodo. In some embodiments, R² is hydrogen. In other embodiments, R² is methyl. In some embodiments, R¹ is hydrogen and R² is hydrogen. In some embodiments, R¹ is hydrogen and R² is methyl. In some embodiments, R¹ is fluoro and R² is hydrogen. In some embodiments, R¹ is iodo and R² is hydrogen.

In some instances, a UAA is located proximal to one or more residues on a target. In some instances, the UAA forms a covalent bond with a residue on the target. In some instances, the residue on the target is an amino acid within a polypeptide chain of the target, such as lysine, tyrosine, or histidine. In some instances, after the targeting domain binds to the target, the UAA is within 5-20, 5-10, 8-20, 10-17, 10-20 or 1-20 angstroms of a residue on the target and forms a covalent bond therewith. In some instances, an unnatural amino acid described herein is incorporated into a peptide chain of the targeting domain. In some instances, an unnatural amino acid described herein is incorporated into a peptide chain of the targeting domain via amide bonds.

### Linkers

In some embodiments, useful functional reactive groups for conjugating or binding a targeting domain to an additional targeting domain described herein include, for example, zero or higher-order linkers. In some instances, a conjugating moiety comprises a functional reactive group that reacts with a linker (optionally pre-attached to a targeting domain, or other portion of a conjugate) described herein. In some embodiments, a linker comprises a reactive group that reacts with a natural amino acid in a targeting moiety described herein.

In various embodiments, the targeting domains are connected or separated by a linker. The linker may be a polypeptide linker. The linker may be expressed via recombinant technology and may be encoded using a nucleic acid sequence in conjunction with the expression of the targeting domains. The linker may link the first targeting domain and the second targeting domain to form a fusion protein. The presence of a linker in the conjugates may allow the targeting domains to function properly without steric interference from the other targeting domain. The linker may be a flexible linker. The flexibility of the linker may allow the targeting domains to adopt independent conformations with minimal interference from the other targeting domain. In some embodiments, the linker is a polypeptide linker comprising, e.g., at least 2, 3, 4, 5, 6, 7, 8, 9, 10, 11, 12, 15, 20, 25, 30, 35, 40, 45, 50, or more amino acids. In some instances, the polypeptide linker comprises at most 2, 3, 4, 5, 6, 7, 8, 9, 10, 11, 12, 15, 20, 25, 30, 35, 40, 45, 50, or less amino acids. In additional cases, the polypeptide linker comprises about 2, 3, 4, 5, 6, 7, 8, 9, 10, 11, 12, 15, 20, 25, 30, 35, 40, 45, or 50 amino acids. In some instances, a polypeptide linker comprises L1. In some instances, a peptide linker comprises (GGGGS)ₓ (SEQ ID NO: 65) or (GGGGSGGGS)ₓ (SEQ ID NO: 56), wherein x is an integer from 1 to 10. In some instances, a peptide linker comprises (GGGGS), wherein x is an integer from 1 to 4.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimide, disulfide, amide, ester, thioester, alkene, cycloalkene, alkyne, triazole, carbamate, carbonate, cathepsin B-cleavable, and hydrazone.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers in some instances are selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

Optionally, multiple targeting domain or modified targeting domain molecules may be joined by a linker polypeptide, wherein said linker polypeptide optionally is 1, 1-2, 1-3, 1-4, 1-5, 1-6, 1-7, 1-8, 1-9, 1-10, 1-11, 1-12 amino acids in length, and longer in length, wherein optionally the N-terminus of one targeting domain is fused to the C-terminus of the linker polypeptide and the N-terminus of the linker polypeptide is fused to the N-terminus of another targeting domain.

### Methods of Treatment

Conjugates described herein may be used to treat conditions and/or diseases. In some instances, the disease comprises a proliferative disease. In some instances, the proliferative disease comprises cancer. In some instances, the cancer comprises tumor cells. In some instances, the cancer comprises solid or liquid tumors. In some instances, conjugates are administered to kill or inhibit growth of a rapidly dividing cell, such as a tumor cell. In some instances, a method of treating a proliferative disease or condition in a subject in need thereof comprises administering to the subject a therapeutically effective amount of a conjugate described herein. In some embodiments, the proliferative disease or condition is a cancer. In some embodiments, the cancer is a solid tumor cancer. In some embodiments, the solid tumor cancer is bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, or prostate cancer. In some instances, the disease comprises PCa (prostate cancer), CRPCa (castration resistant prostate cancer), solid tumors (neovasculature), NSCLC (non-small cell lung cancer), HNSCC (head and neck squamous cell carcinoma), ESCC (esophageal cancer) GC (gastric cancer), CRC (colorectal cancer), SCLC (small cell lung cancer), MPM (mesothelioma), PDAC (Pancreatic ductal adenocarcinoma), ALL (Acute Lymphoblastic Leukemia), AML (Acute Myeloid Leukemia), MDS (Myelodysplastic syndromes), MSI-high tumors, melanoma, DLBCL (diffuse large B cell lymphoma), endometrial cancer, cervical cancer, bladder cancer, BrCa (breast cancer), TNBC (triple negative breast cancer), NE-PCa (Neuroendocrine prostate cancer), GBM (glioblastoma), and RCC (Renal cell carcinoma).

In some instances, tumor cells targeted herein overexpress one or more targets. In some instances, the target comprises surface markers or receptors. In some instances, the target is selected from one or more of 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CCR2, CCR4, CCR7, CD103, CD123, CD13, CD138, CD142, CD147, CD155, CD16, CD166, CD171, CD19, CD2, CD20, CD205, CD206, CD22, CD228, CD24, CD248, CD25, CD30, CD300f, CD33, CD34, CD352, CD36, CD37, CD38, CD40, CD44v6, CD45, CD46, CD47, CD48, CD51, CD56, CD66, CD66, CD70, CD71, CD73, CD74, CD79b, CD8, CD99, CDCP1, CDH17, CDH6, CEA, CEACAM6, cKIT, CLDN18.2, CLDN6, CLDN9, CLL-1, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FLT3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, IL-7RILT-3, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, Macrophage mannose receptor1, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX00IL, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TFR, TIM-1, tissue factor, TM4SF1, TMEFF2, TNFSF12, TNFSF12A, TRA-1-60, TREM2, Trk, TROP-2, TRP1, TSLPR, UCHT1, VCAM-1, VEGF, VEGFR2, VpreB, VPREB1, or xCT. In some instances, the first target comprises 5T4, ADAM-9, AG-7, ALK, ALPP, ALPPL2, ALPV, AMHR2, ASCT2, AXL, BIR, B7-H3, B7-H4, BCMA, C4.4a, CA6, CAIX, CanAg, CAXII, CD123, CD13, CD138, CD142, CD147, CD155, CD166, CD171, CD19, CD2, CD20, CD205, CD22, CD228, CD24, CD248, CD30, CD33, CD34, CD36, CD38, CD44v6, CD45, CD46, CD47, CD48, CD56, CD66, CD70, CD71, CD73, CD74, CD79b, CD99, CDCP1, CDH17, CDH6, CEA, CEACAM6, cKIT, CLDN18.2, CLDN6, CLDN9, cMET, CSP-1, CSPG4, CXCR3, CXCR4, CXCR5, DCLK1, DLK1, DLL1, DLL3, Doppel, DPEP3, DPP4, DR5, DUX4, Dysadherin, EDB fibronectin, EGFR, EGFRviii, EMP2, endo 180, Endoglin, ENPP3, EpCAM, EphA2, EphA3, EphA4, ETBR, FAP, FcRH5, FGFR2, FGFR3, FolRa, GD2, GD3, GloboH, gpA33, GPC-1, GPC3, GPNMB, GPR20, GPRC5d, GRPR, GSPT1, GUCY2C (GCC), HER2, HER3, HLA-DR, ICAM-1, IGF-1R, IL-13Ra2, IL-1RAP, IL-7R, ILT-3, Integrin α₁₀β₁, ITGa Vb3, ITGa Vb6, ITGB4, KAAG1, KIF20A, LAMP-1, Lewis Y antigen, LGR5, LIV-1, LRP5, LRP6, LRRC15, LSR, Ly6E, Ly6G6D, MAGE, MCIR, MerTK, MICA, MICB, MRC2, MSLN, MT1-MMP, MTX7, Muc1, Muc16, NaPi2b, NECTIN4, NKG2DL, NOTCH3rec, NTS1, OX001L, pCadherin, PD-L1, PD-L2, Podocalyxin, PRLR, Prostaglandin F2 Receptor Negative Regulator, PSCA, PSMA, PT1, PTK7, RET, RON, ROR1, ROR2, SAIL, SEA, SEZ6, SIRPa, SLAMF7, SLC44A4, SLITRK6, SSEA-4, SSTR2, STEAP1, STT3, STT4, Survivin, TEM8, TfR, TIM-1, tissue factor, TM4SF1, TMEFF2, TNFSF12, TRA-1-60, Trk, TROP-2, TRP1, TSLPR, UCHT1, VCAM-1, VEGF, VEGFR2, VpreB1, or xCT. In some instances, the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, Eph A4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT. In some instances, the first target comprises 5T4, B7-H3, B7-H4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, PSMA, ROR1, SEZ6, or SLAMF7. In some instances, the first target comprises a lymphocyte antigen. In some instances, the lymphocyte antigen comprises BAFFR, CCR2, CCR4, CCR7, CD103, CD155, CD16, CD2, CD205, CD206, CD25, CD300f, CD34, CD352, CD36, CD37, CD38, CD40, CD46, CD47, CD48, CD51, CD56, CD66, CD70, CD8, CLL-1, CXCR4, FcRH5, FLT3, GPRC5d, HLA-DR, HLA-DR, IL-13Ra2, IL-1RAP, IL-7RILT-3, Ly6E, Ly6G6D, Macrophage mannose receptor 1, MerTK, NKG2DL, PD-L1, PD-L2, SAIL, SIRPa, TFR, TIM-1, TNFSF12A, TREM2, TSLPR, VpreB, or VPREB1.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

In one embodiment, an injectable pharmaceutical composition described herein, is used in the preparation of medicaments for the treatment of diseases or conditions in a mammal that would benefit from administration of any one of the injectable pharmaceutical compositions of the conjugates disclosed. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, wherein the patient's condition does not improve, upon the doctor's discretion the administration of the compounds is administered chronically, that is, for an extended period of time, including throughout the duration of the patient's life in order to ameliorate or otherwise control or limit the symptoms of the patient's disease or condition.

### Methods of Manufacture

Conjugates described herein may be synthesized using in vivo or in vitro methods, or a combination of methods. In some cases, the method is an in vivo method. In some cases, the method is an in vitro method. In some instances, a conjugate comprises a first targeting domain and a second targeting domain, and the first targeting domain comprising an unnatural amino acid is synthesized in vivo, and a second targeting domain is attached in vivo, such as through a fusion protein. In some instances, a conjugate comprises a first targeting domain and a second targeting domain and the first targeting domain comprising an unnatural amino acid is synthesized using in-vitro chemical methods. In some cases, the method is an ex vivo method. In some cases, a conjugate described herein that comprises a natural amino acid mutation or a non-natural amino acid mutation is recombinantly produced or chemically synthesized. In some cases, the first targeting domain, the second targeting domain or both the first and the second targeting domains described herein is produced recombinantly, e.g., by a host cell system or in a cell-free system.

In general, methods of making a target polypeptide that includes a non-standard amino acid are known. In some cases, the aminoacyl tRNA synthetase/tRNA pair cognate to an unnatural amino acid is orthogonal to the cellular components of the cell in which it is used. The orthogonality (and therefore the suitability) of exogenous aminoacyl tRNA synthetase/tRNA pairs is dependent on the type of host organism. Four main orthogonal aminoacyl-tRNA synthetases have been developed for genetic code expansion: the Methanococcus janaschii tyrosyl-tRNA synthetase (MjTyrRS)/tRNA^{CUA} pair, the Escherichia coli tyrosyl-tRNA synthetase (EcTyrRS)/tRNA^{CUA} pair, the E. coli leucyl-tRNA synthetase (EcLeuRS)/tRNA^{CUA} pair, the Methanomethylophilus alvus pyrrolysyl-tRNA synthetase PylRS/tRNA^{CUA} pair, and pyrrolysyl-tRNA synthetase (PylRS)/tRNA^{CUA} (tRNA^{pyl}) pairs from certain Methanosarcina. The PylRS/tRNA^{CUA} pair is orthogonal in bacteria, eukaryotic cells, and animals (see, e.g., Chin, Jason W. “Expanding and reprogramming the genetic code of cells and animals.” Annual review of biochemistry 83 (2014): 379-408).

In some case, the unnatural amino acid (UAA) provided herein is incorporated using a pyrrolysyl-tRNA synthetase (tRNA^{pyl}). The unnatural amino acid (UAA) is introduced on a transfer RNA molecule (tRNA) such that it may be used in translation. However, the attachment of unnatural amino acids to tRNA may not necessarily be accomplished by the naturally occurring aminoacyl-tRNA synthetase. Thus, engineered aminoacyl-tRNA synthetases such as engineered tRNA^{pyl} prepared and selected from a generated tRNA^{pyl} mutant library may be useful for attaching the desired UAA to tRNA so that the desired UAA can be incorporated in mutagenesis.

In some embodiments, the UAA provided herein (e.g., UAA of Formulas (I), (II), (III), (IA), (IB), (IC), (ID), (IE), (IIA), (IIB), or (IV), such as FSY, FSK, and FFY) can be attached to a tRNA using an engineered mutant tRNA^{pyl} variant that is capable of attaching such a UAA. In some embodiments, the mutant tRNA^{pyl} variant comprises a single amino acid mutation as compared to the wildtype tRNA^{pyl}. In other embodiments, the mutant tRNA^{pyl} variant comprises multiple amino acid mutations as compared to the wildtype tRNA^{pyl}. In some embodiments, a library of tRNA^{pyl} variants is prepared and screened by those skilled in the art to select the appropriate tRNA^{pyl} variant for attachment of the desired UAA. In some embodiments, the tRNA^{pyl} variant used to introduce UAA(s) in the conjugates provided herein is a tRNA Pyl variant described in U.S. Pat. Nos. 8,735,093, 9,133,449, WO2020206341, each of which is incorporated by reference herein.

In some embodiments, the mutant pyrrolysyl-tRNA synthetase provided herein comprises at least 5 amino acid residues substitutions within the substrate-binding site of the mutant pyrrolysyl-tRNA synthetase. In some embodiments, the mutant pyrrolysyl-tRNA synthetase provided herein has the amino acid sequence of SEQ ID NO:58. In some embodiments, the mutant pyrrolysyl-tRNA synthetase comprising an amino acid sequence of SEQ ID NO:58. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:58. In some embodiments, the mutant pyrrolysyl-tRNA synthetase provided herein is encoded by the nucleic acid sequence of SEQ ID NO:59. In some embodiments, the mutant pyrrolysyl-tRNA synthetase is encoded by a nucleic acid sequence comprising the sequence of SEQ ID NO:59. In some embodiments, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO:59. In some embodiments, the mutant pyrrolysyl-tRNA synthetase provided herein has the amino acid sequence of SEQ ID NO:61. In some embodiments, the mutant pyrrolysyl-tRNA synthetase comprising an amino acid sequence of SEQ ID NO:61. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:61. In some embodiments, the mutant pyrrolysyl-tRNA synthetase provided herein has the amino acid sequence of SEQ ID NO:61. In some embodiments, the mutant pyrrolysyl-tRNA synthetase comprising an amino acid sequence of SEQ ID NO:61. In some embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:61.

In some instances, sequences associated with mutant pyrrolysyl-tRNA synthetase herein are in Table 2.

TABLE 2
pyrrolysyl-tRNA synthetases

| SEQ ID NO. | DESCRIPTION | SEQUENCE |
|---|---|---|
| 58 | amino acid | MDKKPLNTLISATGLWMSRTGTIHKIKHHEVSRSKI |
| | sequence of | YIEMACGDHLVVNNSRSSRTARALRHHKYRKTCKR |
| | FSYRS | CRVSDEDLNKFLTKANEDQTSVKVKVVSAPTRTKK |
| | | AMPKSVARAPKPLENTEAAQAQPSGSKFSPAIPVST |
| | | QESVSVPASVSTSISSISTGATASALVKGNTNPITSMS |
| | | APVQASAPALTKSQTDRLEVLLNPKDEISLNSGKPF |
| | | RELESELLSRRKKDLQQIYAEERENYLGKLEREITRF |
| | | FVDRGFLEIKSPILIPLEYIERMGIDNDTELSKQIFRV |
| | | DKNFCLRPMLIPNLYNYLRKLDRALPDPIKIFEIGPC |
| | | YRKESDGKEHLEEFTMLTFIQMGSGCTRENLESIITD |
| | | FLNHLGIDFKIVGDSCMVLGDTLDVMHGDLELSSA |
| | | VVGPIPLDREWGIDKPKIGA |
| | | GFGLERLLKVKHDFKNIKRAARSESYYNGISTNL* |
| 59 | nucleic acid | ATGGATAAAAAGCCTTTGAACACTCTGATTTCTG |
| | (DNA) sequence | CGACCGGTCTGTGGATGTCCCGCACCGGCACCAT |
| | of FSYRS | CCACAAAATCAAACACCATGAAGTTAGCCGTTCC |
| | | AAAATCTACATTGAAATGGCTTGCGGCGATCACC |
| | | TGGTTGTCAACAACTCCCGTTCTTCTCGTACCGCT |
| | | CGCGCACTGCGCCACCACAAATATCGCAAAACCT |
| | | GCAAACGTTGCCGTGTTAGCGATGAGGACCTGAA |
| | | CAAATTCCTGACCAAAGCTAACGAGGATCAGACC |
| | | TCCGTAAAAGTGAAGGTAGTAAGCGCTCCGACCC |
| | | GTACTAAAAAGGCTATGCCAAAAAGCGTGGCCCG |
| | | TGCCCCGAAACCTCTGGAAAACACCGAGGCGGCT |
| | | CAGGCTCAACCATCCGGTTCTAAATTTTCTCCGGC |
| | | GATCCCAGTGTCCACCCAAGAATCTGTTTCCGTA |
| | | CCAGCAAGCGTGTCTACCAGCATTAGCAGCATTT |
| | | CTACCGGTGCTACCGCTTCTGCGCTGGTAAAAGG |
| | | TAACACTAACCCGATTACTAGCATGTCTGCACCG |
| | | GTACAGGCAAGCGCCCCAGCTCTGACTAAATCCC |
| | | AGACGGACCGTCTGGAGGTGCTGCTGAACCCAAA |
| | | GGATGAAATCTCTCTGAACAGCGGCAAGCCTTTC |
| | | CGTGAGCTGGAAAGCGAGCTGCTGTCTCGTCGTA |
| | | AAAAGGATCTGCAACAGATCTACGCTGAGGAAC |
| | | GCGAGAACTATCTGGGTAAGCTGGAGCGCGAAAT |
| | | TACTCGCTTCTTCGTGGATCGCGGTTTCCTGGAGA |
| | | TCAAATCTCCGATTCTGATTCCGCTGGAATACATT |
| | | GAACGTATGGGCATCGATAATGATACCGAACTGT |
| | | CTAAACAGATCTTCCGTGTGGATAAAAACTTCTG |
| | | TCTGCGTCCGATGCTGATTCCGAACTTGTACAACT |
| | | ATTTACGTAAACTGGACCGTGCCCTGCCGGACCC |
| | | GATCAAAATATTCGAGATCGGTCCTTGCTACCGT |
| | | AAAGAGTCCGACGGTAAAGAGCACCTGGAAGAA |
| | | TTCACCATGCTGACATTCATTCAGATGGGTAGCG |
| | | GTTGCACGCGTGAAAACCTGGAATCCATTATCAC |
| | | CGACTTCCTGAATCACCTGGGTATCGATTTCAAA |
| | | ATTGTTGGTGACAGCTGTATGGTGTTAGGCGATA |
| | | CGCTGGATGTTATGCACGGCGATCTGGAGCTGTC |
| | | TTCCGCAGTTGTGGGCCCAATCCCGCTGGATCGT |
| | | GAGTGGGGTATCGACAAACCTAAAATCGGTGCGG |
| | | GTTTTGGTCTGGAGCGTCTGCTGAAAGTAAAACA |
| | | CGACTTCAAGAACATCAAACGTGCTGCACGTTCC |
| | | GAGTCCTATTACAATGGTATTTCTACTAACCTGTA |
| | | A |
| 60 | Wild-type amino | MDKKPLNTLISATGLWMSRTGTIHKIKHHEVSRSKI |
| | acid sequence of | YIEMACGDHLVVNNSRSSRTARALRHHKYRKTCKR |
| | Methanosarcina | CRVSDEDLNKFLTKANEDQTSVKVKVVSAPTRTKK |
| | mazei pyrrolysyl- | AMPKSVARAPKPLENTEAAQAQPSGSKFSPAIPVST |
| | tRNA synthetase | QESVSVPASVSTSISSISTGATASALVKGNTNPITSMS |
| | | APVQASAPALTKSQTDRLEVLLNPKDEISLNSGKPF |
| | | RELESELLSRRKKDLQQIYAEERENYLGKLEREITRF |
| | | FVDRGFLEIKSPILIPLEYIERMGIDNDTELSKQIFRV |
| | | DKNFCLRPMLAPNLYNYLRKLDRALPDPIKIFEIGPC |
| | | YRKESDGKEHLEEFTMLNFCQMGSGCTRENLESIIT |
| | | DFLNHLGIDFKIVGDSCMVYGDTLDVMHGDLELSS |
| | | AVVGPIPLDREWGIDKPWIGAGFGLERLLKVKHDF |
| | | KNIKRAARSESYYNGISTNL* |
| 61 | amino acid | MTVKYTDAQIQRLREYGNGTYEQKVFEDLASRDA |
| | sequence of | AFSKEMSVASTDNEKKIKGMIANPSRHGLTQLMND |
| | FSKRS from | IADALVAEGFIEVRTPIFISKDALARMTITEDKPLFKQ |
| | Methanomethylophilus | VFWIDEKRALRPMLAPNLGSVARDLRDHTDGPVKI |
| | alvus | FEMGSCFRKESHSGMHLEEFTMLNLFDMGPRGDAT |
| | | EVLKNYISVVMKAAGLPDYDLVQEESDVYKETIDV |
| | | EINGQEVCSAAVGPTPIDAAHDVHEPWSGAGFGLE |
| | | RLLTIREKYSTVKKGGASISYLNGAKIN |

In some cases, the conjugate comprises a first targeting domain and a second targeting domain and the conjugate is recombinantly produced by a host cell system, for example as a fusion protein comprising the first targeting domain and the second targeting domain. In some cases, the host cell is a eukaryotic cell (e.g., a mammalian cell, an insect cell, a yeast cell, or a plant cell), an archaeal cell, or a prokaryotic cell (e.g., a gram-positive or gram-negative bacterium). In some cases, the eukaryotic host cell is a mammalian host cell. In some cases, the mammalian host cell is a stable cell line, or a cell line that has incorporated the genetic material of interest into its own genome and has the ability to express the product of that genetic material after multiple generations of cell division. In other cases, the mammalian host cell is a transient cell line, or a cell line that has incorporated the genetic material of interest into its own genome and does not have the ability to express the product of the genetic material after multiple generations of cell division.

Exemplary mammalian host cells include 293T cell line, 293A cell line, 293 FT cell line, 293F cell, 293H cell, A549 cell, MDCK cell, CHO DG44 cell, CHO-S cell, CHO-K1 cell, Expi293F cellTM cell, Flp-InTMT-REXTM293 cell line, Flp-InTM-293 cell line, Flp-InTM-3T3 cell line, Flp-InTMBHK cell line, Flp-InTM-CHO cell line, Flp-InTM-CV-1 cell line, Flp-In TMThe Jurkat cell line, FreeStyleTM293-F cells, FreeStyleTMCHO-S cell, Grip TiteTM293MSR cell line, GS-CHO cell line, HepargTM cell, T-REXTMJurkat cell line, Per.C6 cells, T-REXTM-293 cell line, T-REXTM-CHO cell line and T-REXTMHeLa cell line.

In some embodiments, the eukaryotic host cell is an insect host cell. Exemplary insect host cells include Drosophila S2 cell, Sf9 cell, Sf21 cell, and Cellular High Five™ cells.

In some embodiments, the eukaryotic host cell is a yeast host cell. Exemplary yeast host cells include Pichia pastoris yeast strains, such as GS115, KM71H, SMD 1168H, and X-33, and Saccharomyces cerevisiae yeast strains, such as INVSCI.

In some embodiments, the eukaryotic host cell is a plant host cell. In some cases, the plant cell comprises a cell from an alga. Exemplary plant cell lines include strains from Chlamydomonas reinhardtii 137c or Synechococcus elongatus PPC 7942.

In some embodiments, the host cell is a prokaryotic host cell. Exemplary prokaryotic host cells include BL21, Mach1TM, DH10BTM, TOP10, DH5α, DH10BacTM, OmniMax™ MegaX™, DH12STM, INV110, TOP10F′, INVαF, TOP10/P3, ccdB Survival, PIR1, PIR2, Stb12TM, Stb13 TMOr Stb14TM.

In some cases, suitable nucleic acid molecules or vectors for producing the targeting domains described herein include any suitable vector derived from eukaryotic or prokaryotic sources. Exemplary nucleic acid molecules or vectors include vectors from bacterial (e.g., E. coli), insect, yeast (e.g., Pichia pastoris), algal, or mammalian sources. Bacterial vectors include, for example, pACYC177, pASK75, the pBAD series of vectors, the pBADM series of vectors, the pET series of vectors, the pETM series of vectors, the pGEX series of vectors, pHAT2, pMal-C2, pMal-p2, pQE series of vectors, pRSET A, pRSET B, pRSET C, the p TrcHis2 series, pZA31-Luc, pZE21-MCS-1, pFLAG ATS, pFLAG CTS, pFLAG MAC, pFLAG Shift-12C, pTAC-MAT-1, pFLAG CTC or pTAC-MAT-2.

Insect vectors include, for example, pFastBac1, pFastBac DUAL, pFastBac ET, pFastBac HTa, pFastBac HTb, pFastBac HTc, pFastBac M30a, pFastBac M30b, pFastBac, M30c, pVL1392, pVL1393M 10, pVL1393M11, pVL1393M 12, FLAG vectors such as pPolh-FLAG1 or pPolh-MAT2, or MAT vectors such as pPolh-MAT1 or pPolh-MAT 2.

Yeast vectors include, for example, pDESTTM14 a carrier, pDESTTM15 a carrier, pDESTTM17 a carrier, pDESTTM24 carrier, a, pYES-DEST52 vector, pBAD-DEST49Target vector, pAO815 Pichia yeast vector, pFLDI Pichia pastoris vector, pGAPZA, Pichia pastoris C vector, Pichia pastoris pPIC3.5K vector, pPIC 6A, B and Pichia pastoris C vector, pPIC9K vector, pTEF1/Zeo, p YES2 yeast vector, p YES2/CT yeast vector, p YES2/NT A, B and C yeast parent or p YES3/CT yeast vector.

Algal vectors include, for example, pChlamy-4 vectors or MCS vectors.

Mammalian vectors include, for example, transient expression vectors or stable expression vectors. Exemplary mammalian transient expression vectors include p3xFLAG-CMV 8, pFLAG-Myc-CMV19, pFLAG-Myc-CMV 23, pFLAG-CMV 2, pFLAG-CMV 6a, b, c, pFLAG-CMV 5.1, pFLAG-CMV 5a, b, c, p3xFLAG-CMV 7.1, pF-CMV 20, p3xFLAG-Myc-CMV 24, pCMV-FLAG-MAT1, pCMV-FLAG-MAT2, pBICEP-CMV 3, or pBICCMV-4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV14, pFLAG-Myc-CMV 22, p3xFLAG-Myc-CMV 26, pBICEP-CMV 1, or pBICEP-CMV 2.

In some cases, a cell-free system is used to produce a targeting domain described herein. In some cases, cell-free systems comprise a mixture of cytoplasmic and/or nuclear components from cells and are suitable for in vitro nucleic acid synthesis. In some cases, cell-free systems utilize prokaryotic cellular components. In other cases, cell-free systems utilize eukaryotic cell components. Nucleic acid synthesis is achieved in cell-free systems based on, for example, Drosophila cells, Xenopus eggs, archaea or HeLa cells. Exemplary cell-free systems include the E. coli S30 Extract system, the E. coli T7S 30 system or XpressCF and XpressCF+.

Cell-free translation systems variously comprise components such as plasmids, mRNA, DNA, RNA, synthetases, release factors, ribosomes, chaperones, translation initiation and elongation factors, natural and/or unnatural amino acids, and/or other components for protein expression. Such components are optionally modified to improve yield, increase synthesis rate, increase fidelity of the protein product, improve folding or chaperone activity, or incorporate unnatural amino acids. In some embodiments, the unnatural amino acid-containing targeting domains described herein are synthesized using the cell-free translation system described in U.S. Pat. No. 8,778,631, US2017/0283469, US 2018/0051065, US 2014/0315245, or U.S. Pat. No. 8,778,631. In some embodiments, the cell-free translation system comprises a modified release factor, or even one or more release factors are removed from the system. In some embodiments, the cell-free translation system comprises a reduced protease concentration. In some embodiments, the cell-free translation system comprises a modified tRNA having a reassigned codon encoding an unnatural amino acid. In some embodiments, the synthetases described herein for incorporating unnatural amino acids are used in cell-free translation systems. In some embodiments, the tRNA is preloaded with an unnatural amino acid using an enzymatic or chemical process prior to adding the tRNA to the cell-free translation system. In some embodiments, the components for the cell-free translation system are obtained from a modified organism, such as a modified bacterium, yeast, or other organism.

An orthogonal or expanded genetic code can be used to generate the targeting domains described herein, wherein one or more specific codons present in the nucleic acid sequence of a targeting domain are assigned to encode an unnatural amino acid, such that it can be genetically incorporated into a conjugate (e.g., targeting domain) through the use of an orthogonal tRNA synthetase/tRNA pair. The orthogonal tRNA synthetase/tRNA pair is capable of charging a tRNA with an unnatural amino acid, and is capable of incorporating the unnatural amino acid into a polypeptide chain in response to a codon. tRNA synthetase and tRNA may be those described in (or generated using methods described in) Wang et al, “Genetically Encoding Fluorosulfate-L-tyrosine To React with Lysine, Histidine, and Tyrosine via SuFEx in Proteins in Vivo,” J. Am. Chem. Soc. 2018, 140 4995-4999, or Wang et al, “A Genetically Encoded Fluorosulfonyloxybenzoyl-L-lysine for Expansive Covalent Bonding of Proteins via SuFEx Chemistry,” J. Am. Chem. Soc. 2021, 143 10341-10351.

In some cases, the codon is an amber, ochre, an opal codon, or a quadruple codon. In some cases, the codon corresponds to an orthogonal tRNA that will be used to carry the unnatural amino acid. In some cases, the codon is an amber codon. In other cases, the codon is an orthogonal codon.

In some cases, the codon is a quadruple codon, which can be decoded by the orthogonal ribosomal ribo-Q1. In some cases, the quadruple codons are as described in Neumann et al, “Encoding multiple unnatural amino acid analysis of a quadruplet-decoding ribosome,” Nature, 464 (7287): 441-444 (2010).

In some cases, a codon used in the present disclosure is a recoded codon, e.g., a synonymous codon or a rare codon that is replaced with a replacement codon. In some cases, the recoded codons are as described in Napolitano et al, “Emergent rules for codon choice isolated by editing of a ray array code in Escherichia coli,” PNAS, 113 (38): E5588-5597 (2016). In some cases, the recoded codons are as described in Ostrov et al., “Design, synthesis, and testing translated a 57-code gene,” Science 353 (6301): 819. sub. 822 (2016).

### Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, g-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, g-carboxyglutamate, and O-phosphoserine. In aspects, the amino acid side chain may be a non-natural amino acid side chain. In aspects, the amino acid side chain is H,

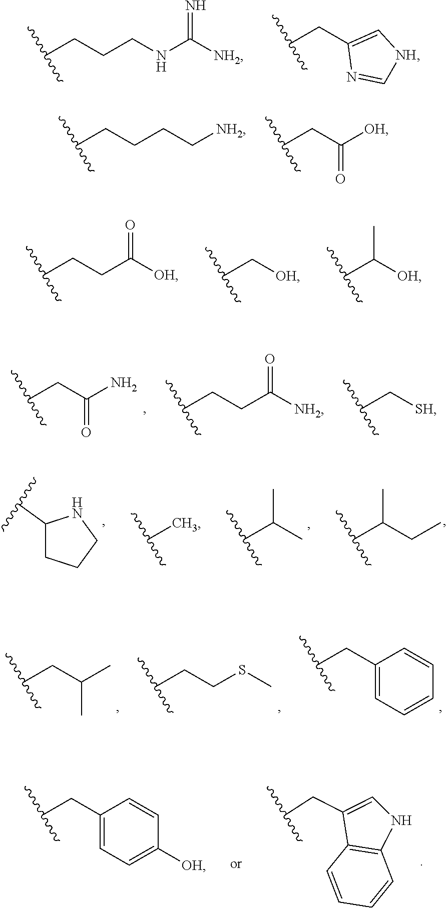

In some embodiments, the unnatural amino acid side chain is

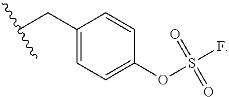 [SMILES: *Cc1ccc(OS(=O)(=O)F)cc1]

In some embodiments, the unnatural amino acid side chain is

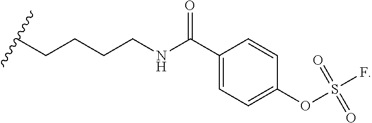 [SMILES: *CCCCNC(=O)c1ccc(OS(=O)(=O)F)cc1]

In some embodiments, the unnatural amino acid side chain is

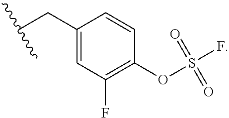 [SMILES: *Cc1ccc(OS(=O)(=O)F)c(F)c1]

The term “non-natural amino acid” or “unnatural amino acid” or “Uaa”, or “non naturally occurring amino acid” refers to the functional substituent of compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. The term may refer to amino acid analogs, synthetic amino acids, and amino acid mimetics which are not found in nature, e.g. amino acid residues containing arylamide, vinyl sulfonamide, sulfonyl fluoride, aryl fluoro sulfate, aryl sulfonyl fluoride, aryl fluorosulfate, and 4-sulfotetrafluorophenyl (STP) esters. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptanecarboxylic acid hydrochloride, cis-6-Amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Bocaminomethyl), benzoic acid, 2-(Boc-amino) octanedioic acid, Boc-4,5-dehydro-Leu-OH (dicyclohexylammonium), Boc-4-(Fmoc-amino)-L-phenylalanine, Boc-P-Homopyr-OH, Boc-(2-indanyl)-Gly-OH, 4-Boc-3-morpholineacetic acid, 4-Boc-3-morpholineacetic acid, Bocpentafluoro-D-phenylalanine, Boc-pentafluoro-L-phenylalanine, Boc-Phe (2-Br)—OH, Boc-Phe (4-Br)—OH, Boc-D-Phe (4-Br)—OH, Boc-D-Phe (3-C1)-OH, Boc-Phe (4-NH₂)—OH, Boc-Phe (3-NH₂)—OH, Boc-Phe (3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Bocpiperazino)-2-[4-(trifluoromethyl)phenyl]acetic acid purum, Boc-P-(2-quinolyl)-Ala-OH, NBoc-1,2,3,6-tetrahydro-2-pyridinecarboxylic acid, Boc-P-(4-thiazolyl)-Ala-OH, Bo-b-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen (Trt)-OH, Fmoc-Phe (2-Br)—OH, Fmoc-Phe (4-Br)—OH, FmocPhe (3,5-F2)-OH, Fmoc-P-(4-thiazolyl)-Ala-OH, Fmoc-P-(2-thienyl)-Ala-OH, 4-(Hydroxymethyl)-D-phenylalanine. In some embodiments, the unnatural amino acid comprises a structure of Formula I:

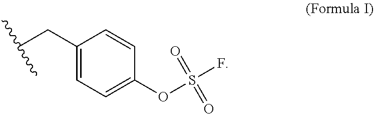 [SMILES: *Cc1ccc(OS(=O)(=O)F)cc1]

In some embodiments, the unnatural amino acid comprises a structure of Formula II:

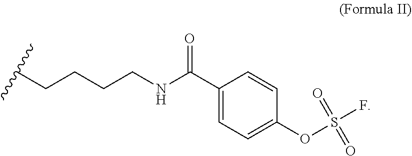 [SMILES: *CCCCNC(=O)c1ccc(OS(=O)(=O)F)cc1]

In some embodiments, the unnatural amino acid is 2-amino-3-(4-((fluorosulfonyl)oxy)phenyl) propanoic acid:

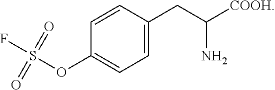 [SMILES: NC(Cc1ccc(OS(=O)(=O)F)cc1)C(=O)O]

In some embodiments, the unnatural amino acid is fluorosulfonyltyrosine (FSY):

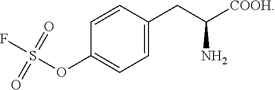 [SMILES: N[C@@H](Cc1ccc(OS(=O)(=O)F)cc1)C(=O)O]

In some embodiments, the unnatural amino acid is N6-(4-((fluorosulfonyl)oxy)benzoyl)lysine:

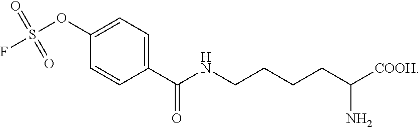 [SMILES: NC(CCCCNC(=O)c1ccc(OS(=O)(=O)F)cc1)C(=O)O]

In some embodiments, the unnatural amino acid is fluorosulfonyloxybenzoyl-L-lysine (FSK):

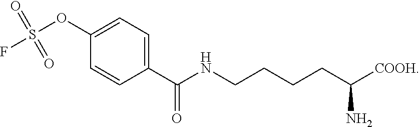 [SMILES: N[C@@H](CCCCNC(=O)c1ccc(OS(=O)(=O)F)cc1)C(=O)O]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

In some embodiments, pyrrolysyl-tRNA synthetase (tRNA^{pyl}) referred to herein is an aminoacyl-tRNA synthetase that catalyzes the reaction for attaching an α-amino acid pyrrolysine or an analogous unnatural amino acid to the cognate tRNA, thereby allowing incorporation of pyrrolysine or analogous unnatural amino acid during proteinogenesis at amber stop codons (i.e., UAG). The wild-type tRNA^{pyl} from Methanosarcina species, which naturally incorporates pyrrolysine, is orthogonal to endogenous tRNAs and aminoacyl-tRNA synthetases in E. coli and eukaryotic cells. Using this pair, and its synthetically evolved derivatives or variants, we and others have directed the efficient incorporation of unnatural amino acids, including post-translationally modified amino acids, chemical handles, and photocaged amino acids, at specific sites in desired proteins in E. coli, yeast, and mammalian cells.

In some embodiments, tRNA^{pyl} described herein includes any recombinant or naturally-occurring form of pyrrolysyl-tRNA synthetase or variants, homologs, or isoforms thereof that maintain tRNA^{pyl} activity (e.g. within at least 30%, 40%, 50%, 60%, 70%, 80%, 90%, 95%, or 100% activity compared to wild-type tRNA^{pyl}). In some embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In some embodiments, the mutant tRNA^{pyl} catalyzes the attachment of an unnatural amino acid (UAA) (e.g., a UAA of Formula I) such as fluorosulfate L-tyrosine (FSY), to a tRNA^{pyl} in order that the unnatural amino acid (UAA) is incorporated.

In some embodiments, the tRNA^{pyl} provided herein is a tRNA^{pyl} derivative or variant that can be engineered by those skilled in the art. In some embodiments, the tRNA Pyl provided herein is a single-stranded RNA molecule containing about 70 to 90 nucleotides which fold via intrastrand base pairing to form a characteristic cloverleaf structure that carries a specific amino acid (e.g., a UAA of Formula I) such as FSY) and matches it to its corresponding codon on an mRNA during protein synthesis.

An “imaging ligand” or a “detectable agent” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷ As, ⁸⁶Y, ⁹⁰Y, ⁸⁹Sr, ⁸⁹Zr, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸ Au, ¹⁹⁹Au, ²¹¹At, ²¹¹Pb, ²¹²B1, ²¹²Pb, ²¹³Bi, ²²³Ra, ²²⁵Ac, ¹⁵³Sm, ¹⁷⁷Lu, ⁹⁰Y, ¹³¹I, ¹⁴⁹Tb, ²¹²Pb/²¹²Bi, ²¹³Bi, ²²⁷Th, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴ Tc, ^{99m}Tc, ¹¹Mo, ¹⁰⁵Pd, ¹⁰⁵Rh, ¹¹¹Ag, ¹¹¹Ln, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶HO, ¹⁶⁹Er, ¹⁷⁵Lu, ¹⁷⁷Lu, ¹⁸⁶Re, ¹⁸⁸Re, ¹⁸⁹Re, ¹⁹⁴I r, ¹⁹⁸ Au, ¹⁹⁹Au, ²¹¹At, ²¹¹P b, ²¹²Bi, ²¹²Pb, ²¹³Bi, ²²³Ra, ²²⁵Ac, ¹⁵³Sm, ¹⁷⁷Lu, ⁹⁰Y, ¹³¹I, ¹⁴⁹Th, ²¹²Pb/²¹²Bi, and ²²⁷Th. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Ala302 of the PylRS protein when the selected residue occupies the same essential spatial or other structural relationship as Ala302 in the PylRS protein. In embodiments, where a selected protein is aligned for maximum homology with the PylRS protein, the position in the aligned selected protein aligning with Ala302 is said to correspond to Ala302. Instead of a primary sequence alignment, a three-dimensional structural alignment can also be used, e.g., where the structure of the selected protein is aligned for maximum correspondence with the PylRS protein and the overall structures compared. In this case, an amino acid that occupies the same essential position as Ala302 in the structural model is said to correspond to the Ala302 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the algorithms can account for gaps and the like. Identity exists over a region that is at least about 25 amino acids or nucleotides in length, or over a region that is 50-100 amino acids or nucleotides in length.

“Antibodies” are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An “antigen binding fragment” as provided herein refers to a polypeptide capable of binding to an antigen and including one or more structural domains of an antibody or fragment thereof. Nonlimiting examples of antibody variants include single-domain antibodies (nanobodies), affibodies (polypeptides smaller than monoclonal antibodies (e.g., about 6 kDA) and capable of binding antigens with high affinity and imitating monoclonal antibodies, monospecific Fab2, bispecific Fab2, trispecific Fab3, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO 97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

A “single-domain antibody” or “nanobody” interchangeably refers to an antibody fragment having a single monomeric variable antibody domain. Like a whole antibody, it is able to bind selectively to a specific antigen. In some embodiments, the single domain antibody is a human or humanized single domain antibody. In some embodiments, the single domain antibody is a camelid single domain antibody.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH₁, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen. Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

### EXAMPLES

### Example 1: Cloning and Expression of FSY-Modified sdAbs

The vector pBAD sequence (Invitrogen #43001) lacking the ORF insert was PCR amplified with the following primers:

forward primer:
(SEQ ID NO: 62)
CTTGGCTGTTTTGGCGGATGAGAGAAGATTTTCAGCCTGATACAG
reverse primer:
(SEQ ID NO: 63)
GTATATCTCCTTCTTAAAGTTAAACAAAATTATTTCTAGCCCAAAAAAAC
GGGTATGGAG

The resulting PCR-amplified vector was gel extracted and purified using a Zymo PCR purification kit (Zymoclean Gel DNA Recovery kits Cat #D4002). The dsDNA sequences for single domain antibodies (sdAbs) and biparatopic constructs were ligated with the PCR-amplified pBAD-backbone and transformed into E. coli DH10b chemically competent cells (Fisher Thermo Scientific™ DH10B Competent Cells; High Efficiency; FEREC0113). Clones were miniprepped and sequence verified using pBAD-forward primer. For constructs incorporating an FSY residue, a TAG codon was engineered into the DNA sequence at the desired position for the amino acid substitution within the open reading frame of the sdAb or biparatopic construct.

### Expression and Purification:

The pEVOL-FSYRS plasmid (Ref: J. Am. Chem. Soc. 2018, 140, 15, 4995-4999) was synthesized by Genscript. This plasmid expresses the engineered Mm FSYRS aminoacyl tRNA synthetase and was transformed into chemically competent DH10b competent cells using standard methods to generate the parental strain FSYRS-DH10b. Individual plasmids encoding each of the single domain antibodies, biparatopic constructs or variants were transformed into chemically competent FSYRS-DH10b using standard methods. Single colonies were inoculated into 2×YT media (Teknova #Y0166) containing 100 μg/ml ampicillin and 5 μg/ml chloramphenicol and grown overnight shaking at 37° C. approximately 220 rpm. Overnight cultures were mixed 1:1 with sterile 50% glycerol and stored at −80° C.

Strains bearing the plasmids encoding sd Abs or biparatopic constructs were cultured in 2×YT media (Teknova #Y0166) in the presence of 100 μg/ml ampicillin and 34 μg/ml chloramphenicol. Prior to induction, cells were grown at a temperature of 37° C. At an OD600 of 0.6, the culture was supplemented with 1 mM FSY and induced by the addition of 0.2% arabinose. At induction, cultures were moved to 25° C. while shaking at 220 RPM overnight, for a total of 16 hours.

Expressions were harvested by pelleting the cells at 2,200×g for 30 minutes at 4° C. Supernatants were removed and cell pellets were weighed and stored at −80° C. Cell pellets were resuspended for lysis by adding B-PER protein extraction reagent (ThermoFisher #78243) at 4 mL/g pellet. The resuspended pellet was allowed to lyse by placing the samples on an orbital shaker at room temperature for 15 minutes at medium speed. The lysed cells were then clarified via centrifugation at 2200×g for 30 minutes. The soluble fraction in the supernatant was removed for further purification.

To purify the soluble fraction, HisPur Ni-NTA resin (ThermoFisher #88222) was used to capture the soluble material from lysates. Resin storage buffer was removed and the resin was equilibrated by batch washing in Wash Buffer (40 mM Sodium Phosphate, pH 7.2, 300 mM NaCl, 20 mM Imidazole). Batch washes were repeated for a total of 50× resin volume. Clarified lysate described above was added to the washed Ni-NTA resin and allowed to bind for 1 hour at room temperature, with constant rotation. After binding, protein-bound resin was batch washed at 50× the resin volume in Wash Buffer to remove unbound contamination. Protein-bound resin was then transferred to a spin column and spun briefly at 700×g to remove remaining the Wash Buffer. Target protein was then eluted using Elution Buffer (40 mM Sodium Phosphate, 7.2, 300 mM NaCl, 500 mM Imidazole). Elution Buffer was added at 2× the resin volume and allowed to incubate at room temperature for 5 minutes. The sample was centrifuged briefly, and the eluted protein was collected in a new tube. The elution procedure was repeated twice using the same conditions. Elution fractions were pooled and quantified by A280 on a Nanodrop 2000, blanking with Elution Buffer. The Ni-NTA purified protein was then concentrated using 0.5 mL, 3 kDa MWCO PES spin filters (ThermoFisher #88512) and buffer exchanged into 1×PBS via repeated dilution at 8-10× volumes of the sample and concentration.

### Example 2: EGFR-Targeted Biparatopic Construct

An EGFR-targeted sd Ab with FSY (C4-109FSY, SEQ ID NO: 17) and a biparatopic construct with two EGFR-targeted sdAbs joined by a linker (C9 (C4-109FSY-L1-C5), SEQ ID NO: 19) were constructed, cloned, and expressed as described in Example 1. The constructs were synthesized with a pelB leader sequence (SEQ ID NO: 15) that was cleaved off from the mature protein, and six C-terminal histidines were used for His-Tag purification. The biparatopic construct C9 contains a GGGGSGGGGS (SEQ ID NO: 14) linker (L1) between the first sdAb C4, and the second sdAb C5.

The FSY-modified sdAbs were assessed for kinetics of EGFR crosslinking. The proteins were incubated with EGFR at an 8:1 molar ratio (EGFR final concentration was 0.125 mg/mL, 1.25 μM). Samples were taken at time points from 0-180 minutes and the percentage of EGFR cross-linked assessed by SDS-PAGE. The crosslinking band percentage was calculated by quantifying the sdAb-EGFR crosslinking band and the EGFR band intensity was quantified using Image J.

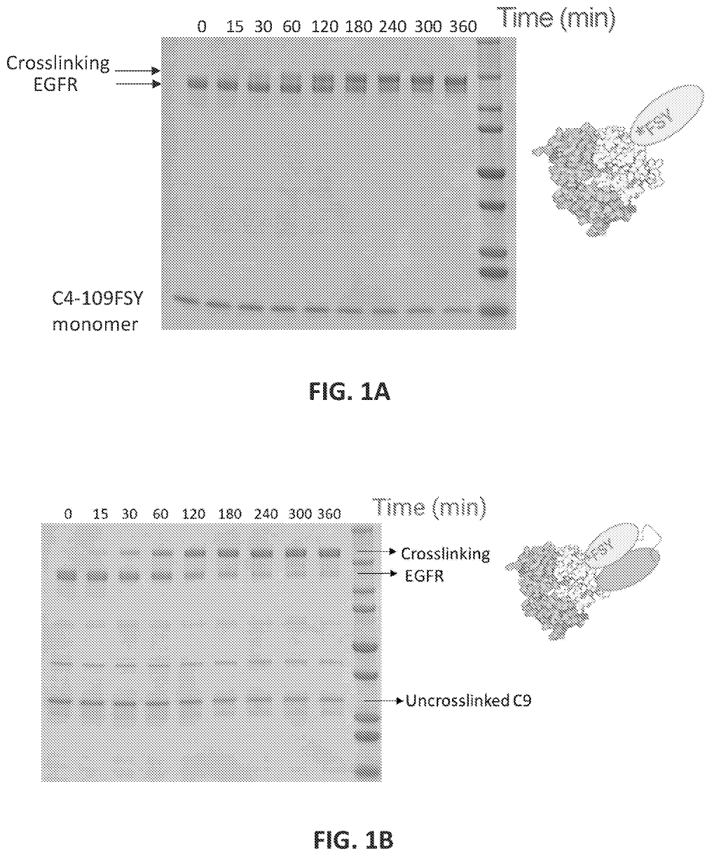

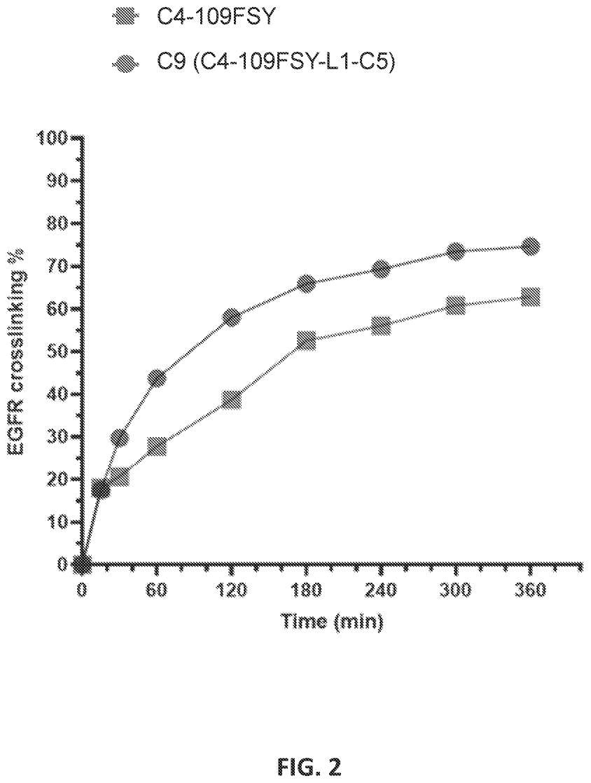

Samples were taken from time zero through 360 minutes. The kinetics of the coupling are shown in FIGS. 1A-1B. The biparatopic construct C9 (C4-109FSY-L1-C5, SEQ ID NO: 19) was compared to a mono-paratopic construct comprising only the sdAb C4 with the FSY modification (C4-109FSY, SEQ ID NO: 17). As shown in FIG. 2, the biparatopic construct C9 coupled to EGFR more rapidly than the monoparatopic C4-109FSY sequence, as measured by a reduced time to half-maximal cross-linking compared to the mono-paratopic construct.

### Example 3: Engineering sdAb C1 to Remove Disulfide Bond in CDR3

A PSMA-targeted sdAb C1 was constructed to express a sdAb with the sequence shown below. The constructs were synthesized with a pelB leader sequence (SEQ ID NO: 15) that was cleaved off from the mature protein, and six C-terminal histidines were used for His-Tag purification.

C1 WT protein sequence (C1) is SEQ ID NO: 1. Additional constructs were made starting from C1 to remove 2 cysteine residues in CDR3 (SEQ ID NO: 7) by modifying these residues to alanine to create C10 (C1-101A/104A, SEQ ID NO: 20).

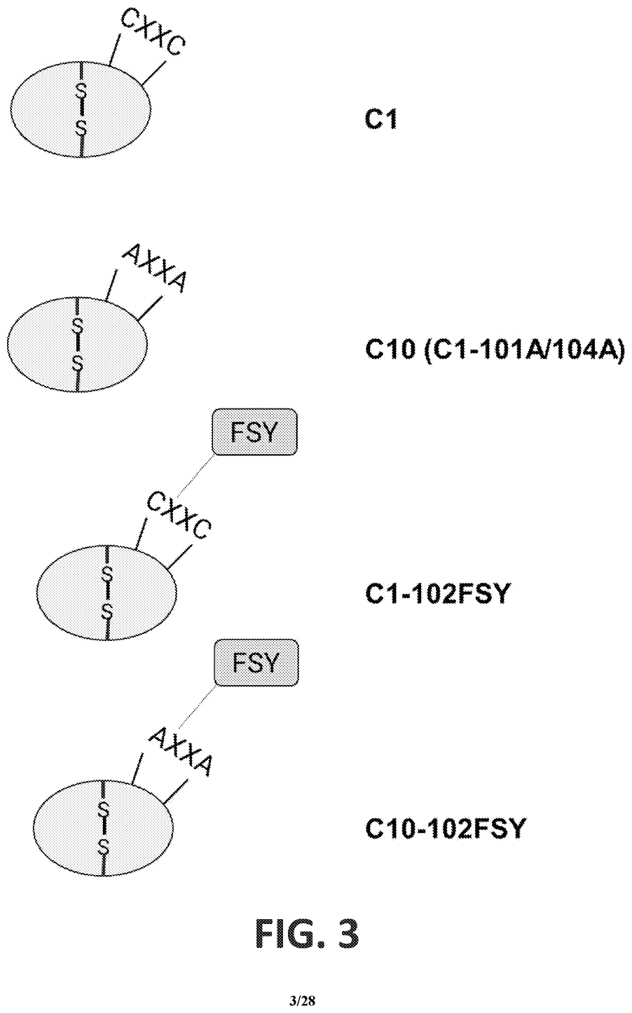

This construct was then further modified to install FSY at residue 102 to create C10-102FSY (C1-101A/104A/102FSY, SEQ ID NO: 4). A diagram of the sdAbs is shown in FIG. 3. The crosslinking capability of these constructs was assessed using the method of Example 2. SDS-PAGE analysis indicated that all the constructs cross-linked at a comparable level under these conditions.

### Example 4: PSMA-Targeted Monoparatopic, Bivalent Construct

The FSY containing PSMA-targeted sdAb C10-102FSY (SEQ ID NO: 4 from Example 3) was cloned and expressed as described in Example 1. A bivalent construct C11-102FSY (C10-102FSY-L1-C10, SEQ ID NO: 21) was created by linking the C10_102FSY from Example 3 with an additional copy of the C10 (SEQ ID NO: 20, without FSY). The construct was designed by adding a GGGGSGGGGS (SEQ ID NO: 14) linker (L1) between the two sdAb amino acid sequences. The bivalent construct was then cloned into pBAD vector and expressed as described in Example 1. The constructs were synthesized with a pelB leader sequence (SEQ ID NO: 15) that was cleaved off from the mature protein, and C-terminal six histidines were used for His-Tag purification.

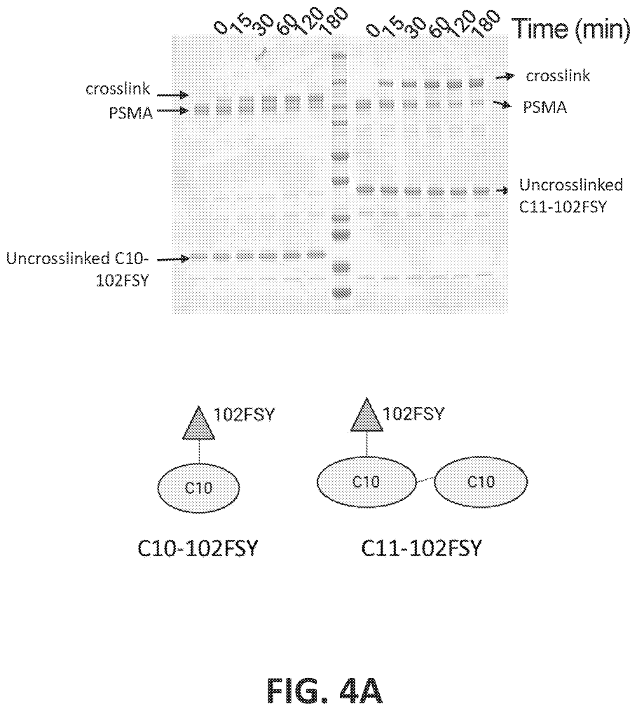

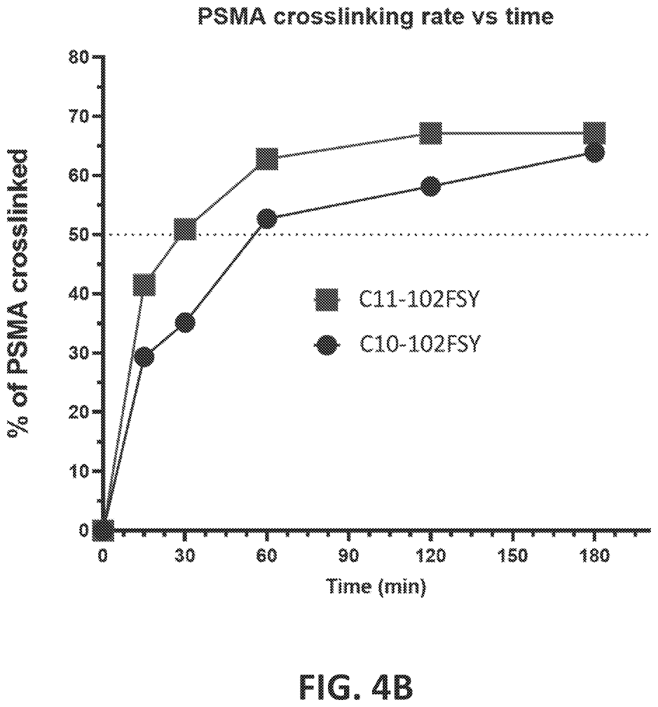

The monoparatopic sdAb and biparatopic constructs were assessed for cross-linking to PSMA by SDS-PAGE and over a time course following the methods provided in Example 2. As shown in FIGS. 4A-4B, the biparatopic construct provided faster coupling as compared to the sdAb construct.

### Example 5: Construction of Bispecific Immune Cell Engagers

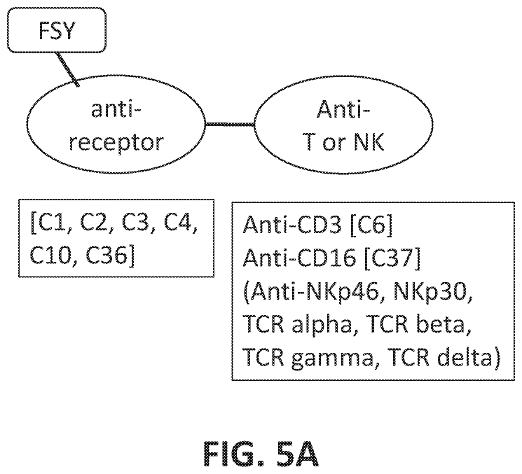

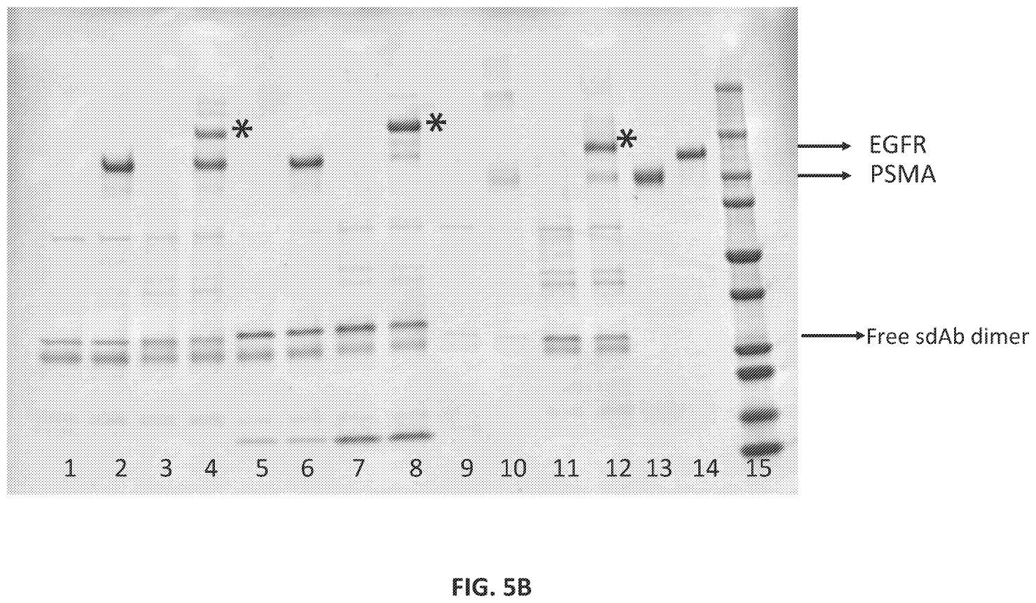

Bispecific T cell engagers were constructed having a sdAb that binds a target (“target sdAb”) joined by a linker to a sdAb targeted to either CD3 or CD16 (“engager sdAb”). In each case, the target sdAb contained the incorporated FSY. Exemplary structures are shown in FIG. 5A.

A bispecific construct C12 (C4-L6-C6, SEQ ID NO: 23) or C13 (C4-109FSY-L6-C6, SEQ ID NO: 24) was created by linking the C4 from Example 2 with an additional copy of the C6 recruiting T cells (C6, SEQ ID NO: 22, without FSY). The construct was designed by adding a GGGGSGGGS (SEQ ID NO: 56) linker between the two sdAb amino acid sequences. The bispecific construct was then cloned into pBAD vector and expressed as described in Example 1. The constructs were synthesized with a pelB leader sequence (SEQ ID NO: 15) that was cleaved off from the mature protein, and C-terminal six histidines were used for His-Tag purification.

A bispecific construct C14 (C4-L2-C7, SEQ ID NO: 26) or C15 (C4-109FSY-L2-C7, SEQ ID NO: 27) was created by linking the C4-109FSY from Example 2 with an additional sdAb, C7 (SEQ ID NO: 25), an NK cell binding sequence lacking FSY. The construct was designed by adding a 3× (G4S) (GGGGSGGGGSGGGGS (SEQ ID NO: 31) linker (L2) between the two sdAb amino acid sequences. The bispecific construct was then cloned into pBAD vector and expressed as described in Example 1. The constructs were synthesized with a pelB leader sequence (SEQ ID NO: 15) that was cleaved off from the mature protein, and six C-terminal histidines were used for His-Tag purification.

A bispecific construct C16 (C2-L6-C6, SEQ ID NO: 28) or C18 (C2-54FSY-L6-C6, SEQ ID NO: 30) was created by linking C17 (C2-54FSY, SEQ ID NO: 29) with an additional sdAb C6 (SEQ ID NO: 22), a T cell binding sequence lacking FSY. The construct was designed by adding the L6 linker (SEQ ID NO: 56) between the two sdAb amino acid sequences. The bispecific construct was then cloned into pBAD vector and expressed as described in Example 1. The constructs were synthesized with a pelB leader sequence (SEQ ID NO: 15) that was cleaved off from the mature protein, and six C-terminal histidines were used for His-Tag purification.

The bispecific FSY-modified sdAbs were assessed for crosslinking with EGFR and PSMA respectively. The proteins were incubated with EGFR or PSMA at an 8:1 molar ratio (EGFR final concentration was 0.125 mg/mL, 1.25 μM). Samples were incubated in 1×PBS, pH 7.4, at 370C, overnight. The percentage of EGFR or PSMA cross-linked was assessed by SDS-PAGE. (FIG. 5B). 1. C12 (C4-L6-C6); 2. C12+EGFR; 3. C13 (C4-109FSY-L6-C6); 4. C13+EGFR 5. C14 (C4-L2-C7); 6. C14+EGFR; 7. C15 (C4-109FSY-L2-C7); 8. C15+EGFR 9. C16 (C2-L6-C6); 10. C16+PSMA; 11. C18 (C2-54FSY-L6-C6); 12. C18+PSMA; 13. PSMA; 14. EGFR; 15. Ladder.

Bispecific constructs C20 (C37-L2-C35), C21 (C37-L1-C35), C22 (C37-L3-C35), C23 (C37-L4-C35), and C24 (C37-L5-C35) were created by linking an anti-CD16a sdAb, C37 (SEQ ID NO: 52) with an anti-PSMA sdAb-FSY (SEQ ID NO: 50) using a 2× (G4S) (L1: GGGGSGGGGS (SEQ ID NO: 15) linker, a 3× (G4S) (L2: GGGGSGGGGGGGGS (SEQ ID NO: 31) linker, a 4× (G4S) (L3: GGGGSGGGGSGGGGSGGGGS (SEQ ID NO: 32)) linker, a 5× (G4S) (L4: GGGGSGGGGSGGGGSGGGGSGGGGS (L4; SEQ ID NO: 33)) linker, or an SP (L5: SPSTPPTPSPSTPP (SEQ ID NO: 34)) linker, respectively. Non-crosslinking bispecific construct, C25 (C37-L3-C36) was also created by linking the anti-CD16a sdAb, C37 (SEQ ID NO: 52) with an anti-PSMA sdAb-TYR (SEQ ID NO: 51) with a 4× (G4S) (L3) linker. The bispecific constructs were then cloned into pBAD vectors and expressed as described in Example 1. The constructs were synthesized with a pelB leader sequence (SEQ ID NO: 15) that was cleaved off from the mature protein, and six C-terminal histidines were used for His-Tag purification.

The purified FSY-containing bispecific sdAb constructs were incubated with PSMA (+PSMA) (Sino Biological; Cat #15877-H07H) at a molar ratio of ˜8:1 in 1×PBS (pH 7.4), or without PSMA (−PSMA; control), at 37° C. for 5 min. Following incubation, the reactions were quenched with SDS-Laemmli loading dye (1× final concentration with 100 mM DTT), heated to 95° C. for 5 min, and analyzed by SDS-PAGE (4-20% Mini-PROTEAN® TGX™) with Coomassie blue staining.

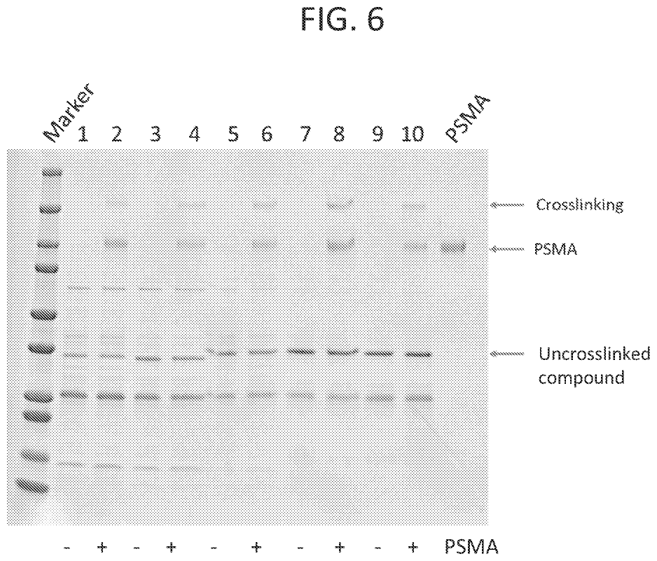

As shown in FIG. 6, each bispecific variant was able to rapidly crosslink with PSMA irrespective of linker length or composition within 5 min. FIG. 6 depicts an SDS-PAGE analysis of various constructs provided herein. Lanes 1-12 are labeled as follows: 0. Ladder; 1. C20 (C37-L2-C35) no PSMA; 2. C20 (C37-L2-C35) with PSMA; 3. C21 (C37-L1-C35) no PSMA; 4. C21 (C37-L1-C35) with PSMA; 5. C22 (C37-L3-C35) no PSMA; 6. C22 (C37-L3-C35) with PSMA; 7. C23 (C37-L4-C35) no PSMA: 8. C23 (C37-L4-C35) with PSMA; 9. C24 (C37-L5-C35) no PSMA; 10. C24 (C37-L5-C35) with PSMA; 11. PSMA. Bands corresponding to EGFR, PSMA, and free sdAb dimer are labeled. Crosslinked species are marked with an asterisk

### Example 6: Binding Confirmation and Kinetic Binding Characterization of Bispecific Engagers Variants to Recombinant Human CD16a and Recombinant Human PSMA

Kinetic binding experiments to recombinant antigens by bio-layer interferometry on an Octet instrument were performed using the conjugates generated in Example 6. For some conjugates, a qualitative experiment was performed using a single concentration (or estimated concentration on intermediate purity samples) to confirm binding to antigen and approximate affinity range. For other conjugates, an experiment to characterize the kinetic binding profile and affinity (kₒₙ, k_{off}, and K_{D}) was performed using a dilution series of highly purified conjugates. Binding experiments evaluated antigen binding to recombinant human CD16a and PSMA antigens.

### PSMA Binding

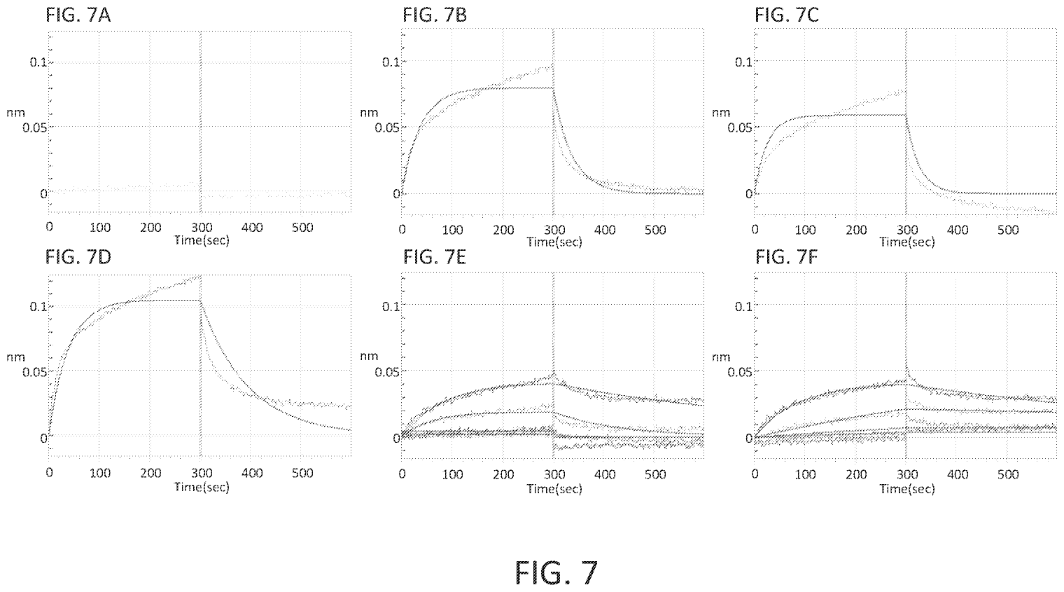

To evaluate PSMA binding, PSMA-hFc (Acro Biosystms Cat. No. PSA-H5264) was immobilized on anti-human capture biosensors (Sartorius Cat. No. 18-5060) and binding to test proteins diluted to the estimated affinity range was carried out in 10 mM sodium citrate phosphate pH 5.7 150 mM sodium chloride with 0.01% PS20. An initial curve was collected and presented in FIG. 7A. Binding of C21 (SEQ ID NO: 36) (FIG. 7B), C20 (SEQ ID NO: 35) (FIG. 7C), C22 (SEQ ID NO: 37) (FIG. 7D), C23 (SEQ ID NO: 38) (FIG. 7E), and C24 (SEQ ID NO: 39) (FIG. 7F) conjugates was tested at concentrations of about 150 nM or a range of concentrations (150 nM to 500 nM). As shown in FIGS. 7A-7F, all four constructs with different number of G4S repeats in the linker (FIGS. 7B-7E), and the construct with the SP linker (L5) (FIG. 7F) bound to PSMA at pH 5.7 with appreciable signal above baseline.

### CD16a Binding

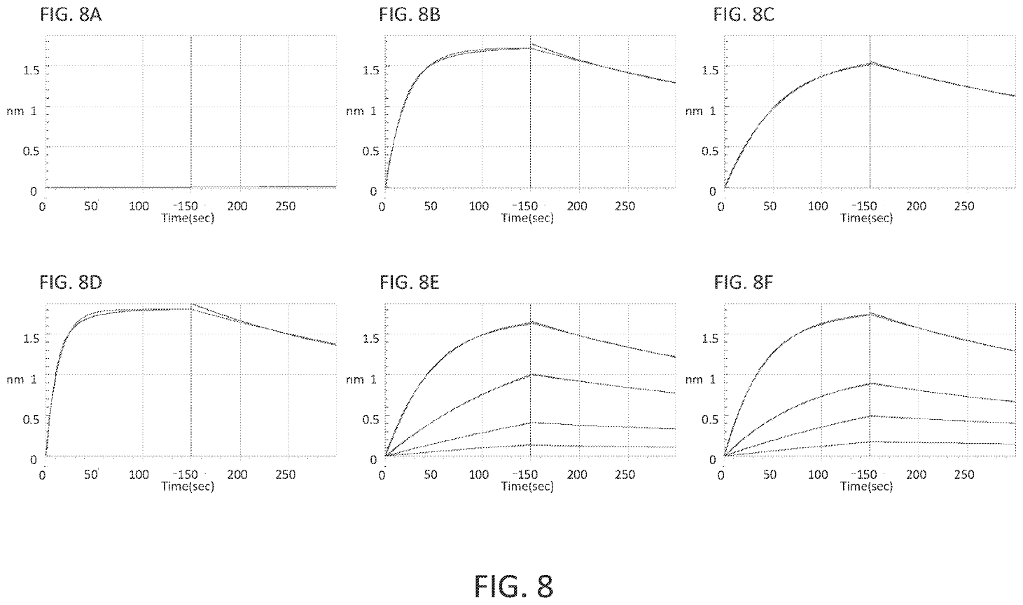

CD16a binding was evaluated by immobilizing biotin-labeled CD16a (Acro P/N CDA-H82E8) on streptavidin biosensors (Sartorius P/N 18-5019) and binding to test proteins diluted to the estimated affinity range was carried out in phosphate or HEPES-buffered saline with 0.01% PS20 at pH 7.4. (A) An initial baseline was collected prior to introducing any constructs was collected. (B) Binding of C21 (SEQ ID NO: 36) was tested at 180 nM; (C) C20 (SEQ ID NO: 35) was tested at 150 nM; (D) C22 (SEQ ID NO: 37) was tested at 500 nM; (E) C23 (SEQ ID NO: 38) was tested at 150 nM, 50 nM, 16.7 nM, and 5.6 nM; and (F) C24 (SEQ ID NO: 39) was tested at 150 nM, 50 nM, 16.7 nM, 5.6 nM. Kinetic binding signal (gray) and 1:1 binding model fit curves (black) of bispecific constructs binding to the immobilized CD16a. As shown in FIGS. 8A-8F, all five constructs bound to CD16a with estimated affinities in the low nanomolar range.

### Kinetic Binding of NK Cell Bispecific Constructs to PSMA at pH 5.7 and 7.4

Compound C22 with FSY (SEQ ID NO: 37) and a non-covalent construct C25 (SEQ ID NO: 40) containing a tyrosine (TYR) instead of FSY were generated, expressed and purified as described in Example 6. The constructs were further purified by size exclusion chromatography (SEC) and chromatography using nC-slyD-A1 resin to generate bispecific constructs at more than 90% purity. The constructs were then evaluated in a titration series to determine the affinity of each binding domain for its respective antigen.

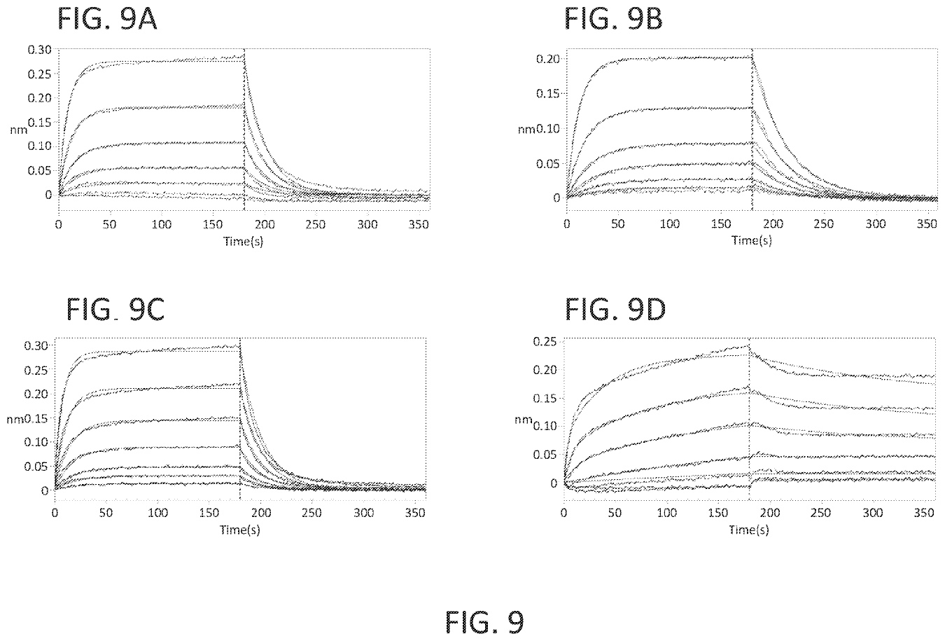

Biotinylated his-tagged human PSMA was immobilized on streptavidin sensors as described above, and binding was evaluated at pH 5.7 or 7.4. Kinetic binding signal and model fit curves (smooth, black) were generated by measuring binding of C25 and C22 to the immobilized PSMA at a 1:2 dilution series from 2 μM to 21.25 nM in pH 5.7 (FIGS. 9A and 9C, respectively) or pH 7.4 (FIGS. 9B and 9D, respectively) buffer. For the non-covalent, control construct, C25, binding to PSMA was nearly identical between pH 5.7 and 7.4 (FIGS. 9A and 9B, respectively), while the FSY-containing bispecific construct, C22, displayed a clear change in the binding kinetics associated with crosslinking at pH 5.7 and 7.4 (FIGS. 9C and 9D, respectively). At pH 7.4 (FIG. 9D), the association curve of the FSY-containing bispecific construct displayed a biphasic rather than 1:1 kinetic curve, and the apparent dissociation was reduced. AtpH 5.7 (FIG. 9C), where the covalent crosslinking (or covalent binding) was considerably reduced, these effects were diminished allowing measurement of only the binding event and calculation of the domain affinity.

The purified constructs were also evaluated for binding CD16a, immobilized on streptavidin biosensors as previously described. The purified CD16a sdAb domain alone was also evaluated as a control. Kinetic binding signal and model fit curves generated from the binding of the molecules at dilutions from 200 nM to 3.125 nM. Concentrations above saturation were not included in fitting analysis.

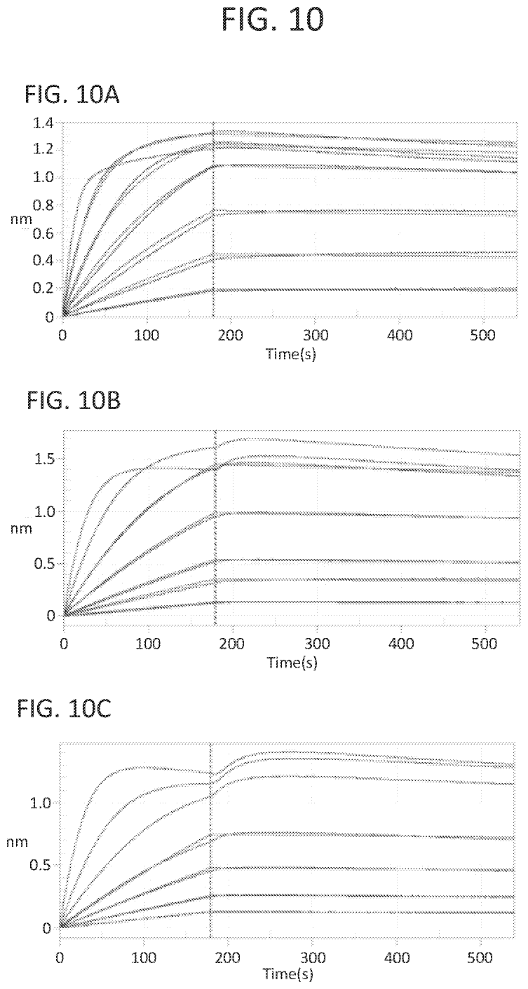

As shown in FIGS. 10A-10C, the kinetic binding to CD16a was similar for the CD16a sdAb domain alone (C37; FIG. 10A), C25 (FIG. 10B) and C22 (FIG. 10C) conjugates, suggesting the conjugation did not impair the binding of the CD16a engaging component of the conjugates.

The determined kinetic binding parameters of C25 and C22 are provided in Table 3. The results show similar binding to PSMA at pH 5.7 and CD16a at pH 7.4, but a considerable difference in the binding mode to PSMA at pH 7.4. These results indicate the difference in the construct binding mode to PSMA at pH 7.4 is due to crosslinking of the anti-PSMA sdAb to its target.

TABLE 3
Kinetic Binding Parameters derived from BLI experiments

| | PSMA | PSMA | CD16a |
|---|---|---|---|
| | kₒₙ, k_{off}, K_{D} | kₒₙ, k_{off}, K_{D} | kₒₙ, k_{off}, K_{D} |
| Construct | at pH 5.7 | at pH 7.4 | at pH 7.4 |
| C22 | 4.07 × 10⁴ M⁻¹ | 1.28 × 10⁴ M⁻¹ | 5.88 × 10⁴ M⁻¹ |
| | s⁻¹ | s⁻¹ (non 1:1 | s⁻¹ |
| | 4.65 × 10⁻² s⁻¹ | binding) | 8.69 × 10⁻⁵ s⁻¹ |
| | 1.14 × 10⁻⁶ M | <1 × 10⁻⁷ s⁻¹ | 1.48 × 10⁻⁹ M |
| | | Sub-nanomolar | |
| C25 | 3.08 × 10⁴ M⁻¹ | 2.59 × 10⁴ M⁻¹ | 1.37 × 10⁵ M⁻¹ |
| | s⁻¹ | s⁻¹ | s⁻¹ |
| | 4.83 × 10⁻² s⁻¹ | 3.00 × 10⁻² s⁻¹ | 1.52 × 10⁻⁴ s⁻¹ |
| | 1.57 × 10⁻⁶ M | 1.16 × 10⁻⁶ M | 1.11 × 10⁻⁹ M |

### Example 7: ADCC Activity Induced by NK Cell Bispecific Constructs

An ADCC reporter assay was employed to examine the impact of covalent linkage to target cells on the ability for bivalent constructs to promote T-cell activity in the presence of target cells expressing different levels of PSMA. PC3pip cells (as described in U.S. Pat. No. 9,713,649 B2, the entirety of which is incorporated by reference herein), expressing a high density of PSMA and 22Rv1 cells (ATCC CRL-2505), expressing a low density of PSMA, were seeded at 12,500 cells/well in 96-well black clear bottom plates in 100 μL RPMI-1640, 10% FBS and incubated 24 hr at 37° C., 5% CO₂ to allow cells to adhere to the plate.

The following day, the culture medium was removed and C25 and C22 bispecific constructs, generated and purified to more than 90% purity as described above, or monospecific anti-CD16a sdAb (C37), anti-PSMA sdAb (TYR) (C36), anti-PSMA (FSY) (C35), or endotoxin control sdAb were added at 10 concentration points of 3-fold serial dilutions starting at a final concentration of 120 nM in ADCC assay buffer (RPMI 1640, 4% low IgG FBS). For further comparison, treatment with monoclonal anti-PSMA antibody, J591, and human IgG isotype control antibody were also tested at 10 concentration points of 3-fold serial dilutions starting at a final concentration of 30 nM in ADCC assay buffer. For cells receiving continual treatment, some medium was removed, followed by addition of the test articles and Jurkat CD16a V158 ADCC reporter cells from the Promega ADCC reporter bioassay kit (Promega, G7010), the latter at a 6:1 effector cell:target cell (E:T) ratio in fresh medium. The cell and test article mixture was then incubated at 37° C. for 24 hrs. For the 4 hr wash out treatment, some medium was removed and the test articles were added with some fresh medium and incubated for 4 hr at 37° C. After the 4 hr incubation, the cells were washed with ADCC assay buffer and then the reporter cells were added at a 6:1 effector cell:target cell (E:T) ratio and then incubated at 37° C. for 20 hrs. For specificity of signal induction, wells with target cells alone (plate background), effectors cells plus target cells without test articles (effector cell background), and effector cells plus the highest concentration of each test article were included (target-independent NK reporter activation).

BioGlo assay buffer and substrate were added to each well, and the luminescence was read on a Victor X5 plate reader (Perkin Elmer) after incubating at room temperature for 5-30 minutes and shaking on a plate shaker for 1 minute. Fold induction was calculated by dividing the background subtracted Relative Light Units (RLU) of treated wells by the background subtracted RLU of non-treated wells. (Fold induction=RLU (induced-background)/RLU (no test article control/plate background). Data were plotted, and the curve fit and EC50 were calculated using ‘log (agonist) vs. response−variable slope (four parameters).

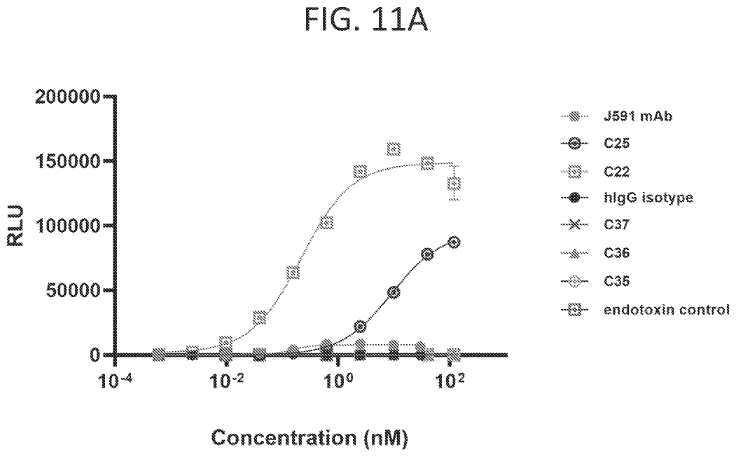

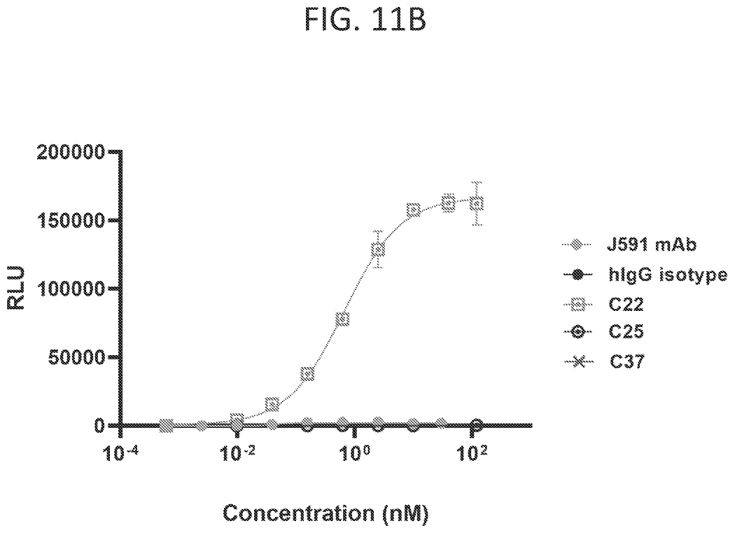

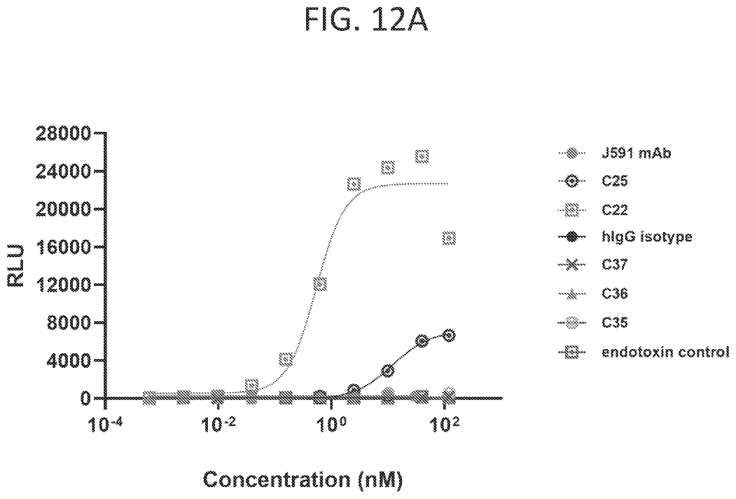

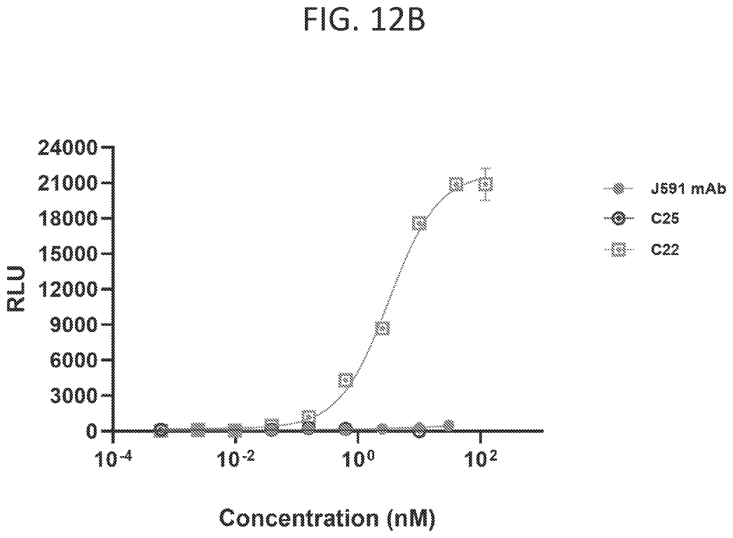

Activation of CD16a-expressing reporter cells following incubation with high PSMA density target cells, PC3 pip, treated with increasing concentrations of the test articles continuously for 24 hr or for 4 hr followed by a washout is plotted in FIGS. 11A and 11B, respectively with a table (Table 4 and Table 5, respectively) of the corresponding EC50 data. Activation of CD16a-expressing reporter cells following incubation with low PSMA density target cells, 22Rv1, treated with increasing concentrations of the test articles continuously for 24 hr or for 4 hr followed by a washout is plotted in FIGS. 12A and 12B, respectively with a table (Table 6 and Table 7, respectively) of the corresponding EC50 data.

TABLE 4
EC₅₀ parameters of various constructs tested
against high-PSMA-expressing PC3pip cells

| | PC3pip 24 h continuous | | | | | | | |
|---|---|---|---|---|---|---|---|---|
| | | | | | | | | Endotoxin |
| | J591 | C25 | C22 | hIgG | C37 | C36 | C35 | control |
| EC₅₀ | 0.1599 | 9.198 | 0.2194 | N/A | N/A | N/A | N/A | N/A |
| EC₅₀ | 0.08478 to | 8.084 to | 0.1487 to | N/A | N/A | N/A | N/A | N/A |
| Range | 0.2184 | 10.66 | 0.3181 | | | | | |
| R- | 0.9484 | 0.9996 | 0.9830 | N/A | N/A | N/A | N/A | N/A |
| squared | | | | | | | | |

TABLE 5
EC₅₀ parameters of various constructs tested against high-PSMA-
expressing PC3pip cells with intermediate washout after 4 hours

| | PC3pip 4 h washout, 24 h endpoint | | | | | | | |
|---|---|---|---|---|---|---|---|---|
| | | | | | | | | Endotoxin |
| | J591 | C25 | C22 | hIgG | C37 | C36 | C35 | ctrl |
| EC₅₀ | 0.04400 | 0.1413 | 0.6666 | N/A | N/A | N/A | N/A | N/A |
| EC₅₀ | 0.01705 to | wide | 0.5271 to | N/A | N/A | N/A | N/A | N/A |
| Range | 0.09297 | | 0.8437 | | | | | |
| R- | 0.8500 | 0.3503 | 0.9936 | N/A | N/A | N/A | N/A | N/A |
| squared | | | | | | | | |

As shown in FIG. 11A, 24 hr. continual exposure of high PSMA density target cells (PC3 pip cells) to the bispecific constructs generated robust and dose-dependent induction of reporter cell signal, but no signal from negative control molecules demonstrating dependency of signal on antigen expression. The FSY-modified bispecific construct generated both greater signal induction and greater potency as compared to the TYR containing construct. The differences observed between the FSY and TYR bispecific constructs were amplified in the 4-hour washout experiments (FIG. 11B), where the FSY-modified construct generated similar maximal signal and slightly reduced potency compared to the 24-hour continual treatment, but the TYR construct lost virtually all activity.

As shown in FIG. 12A, 24 hr. continual exposure of low PSMA density target cells (22Rv1 cells) to the bispecific constructs also generated dose-dependent induction of reporter cell signal. However, the maximum RLUs were considerably lower for both the FSY and TYR-containing constructs. These results correlate with the lower antigen density and are described in Table 6. No signal was observed for any of the negative control molecules. Similar to the observations with the PC3 pip model, the differences between the FSY and TYR constructs were amplified in the 4-hour washout experiments (FIG. 12B), where the FSY-modified construct generated similar maximal signal and slightly reduced potency compared to the 24-hour continual treatment, the TYR construct lost virtually all activity. The results are described in Table 7.

Together, these results demonstrate that both arms of the bispecific constructs are able to induce reporter cell activation in response to the target cells. Further, across a range of receptor densities, FSY-modified conjugates have superior ability to engage and activate the CD16a-expressing Jurkat NFAT reporter cells in a dose and target-dependent manner.

TABLE 6
EC₅₀ parameters of various constructs tested against low-PSMA-expressing 22Rv1 cells

| | 22Rv1 24 h continual | | | | | | | |
|---|---|---|---|---|---|---|---|---|
| | | | | | | | | Endotoxin |
| | J591 | C25 | C22 | hIgG | C37 | C36 | C35 | ctrl |
| EC₅₀ | Unstable | 12.11 | 0.5398 | 0.01129 | >500 | Unstable | Unstable | 13.15 |
| EC₅₀ | Wide | 9.869 to | 0.1916 to | N/A | Wide | Wide | Wide | Wide |
| Range | | 15.75 | 1.207 | | | | | |
| R- | −0.2766 | 0.9919 | 0.9525 | 0.3410 | N/A | N/A | N/A | 0.1028 |
| squared | | | | | | | | |

TABLE 7
EC₅₀ parameters of various constructs tested against low-PSMA-
expressing 22Rv1 cells with intermediate washout after 4 hours

| | 22Rv1 4 h washout, 24 h endpoint | | | | | | | |
|---|---|---|---|---|---|---|---|---|
| | | | | | | | | Endotoxin |
| | J591 | C25 | C22 | hIgG | C37 | C36 | C35 | ctrl |
| EC50 | >500 | 62.61 | 3.186 | N/A | N/A | N/A | N/A | N/A |
| EC50 | wide | wide | 2.647 to | N/A | N/A | N/A | N/A | N/A |
| Range | | | 3.866 | | | | | |
| R | 0.5969 | 0.2079 | 0.9947 | N/A | N/A | N/A | N/A | N/A |
| squared | | | | | | | | |

### Example 8: Bispecific Conjugate Induction of Primary Human NK Cell-Mediated Cytotoxicity Against PSMA-Expressing Cells

The impact of covalent linkage to target cells on the ability for heterodimeric bivalent constructs to induce primary human NK cell-mediated cytotoxicity of target cells expressing high levels of PSMA was tested.

Human primary NK cells were isolated from fresh PBMCs using an NK Cell Isolation Kit (Miltenyi Biotech Cat no. 130-092-657) according to the manufacturer's protocol. Purity of the isolated NK cells was evaluated by flow cytometry. The total purity of the enriched NK cells was determined to be >95%, and majority of the isolated cells were of CD56dim (i.e., cytotoxic phenotype).

PC3 pip cells (as described in U.S. Pat. No. 9,713,649 B2, the entirety of which is incorporated by reference herein), expressing a high density of PSMA, were seeded 10,000 cells/well in 96-well black clear bottom plates in 100 μL RPMI 1640, 10% FBS and incubated 24 hr at 37° C., 5% CO2 to allow cells to adhere to the plate.

For continual treatment with test article, the following day, the culture medium was removed, and C25 and C22 were generated and purified to more than 90% purity as described above. C37 monospecific anti-CD16a sdAb (as a control for target-independent NK cell activation) was also generated and purified as described above. Each of C25, C22, and C37 were added at 9 concentration points of 4-fold serial dilutions, to final concentrations ranging between 100 and 0.0015 nM in RPMI-1640 medium supplemented with 10% heat inactivated FBS (cRPMI). Each concentration was tested in duplicate. A control without a test article was also included. 100,000 primary NK cells in cRPMI and human IL-2 (hIL-2) were added to reach the final hIL-2 concentration of 25 ng/mL. The final volume was approximately 100 μL. The plates were subsequently incubated for 20 hr at 37° C., 5% CO₂.

For the 5 h washout of the test articles, the culture medium was removed from each well, and replaced with the diluted test articles in duplicate. The plates were incubated for 5 hours at 37° C., 5% CO₂, followed by careful removal of all medium and a single wash with 100 μL of cRPMI and subsequent media removal. 100,000 primary NK cells in a final volume of 100 μL CRPMI with hIL-2 (final concentration 25 ng/mL) (E:T of 10:1). All plates were subsequently incubated for 20 hr at 37° C., 5% CO₂.

The following day, the plates were removed from the incubator and cytotoxicity was determined by CellTiter-Glo luminescent cell viability assay (Promega). Luminescence was measured using a ClarioStar (BMG Labtech) reader. Cell Lysis values were calculated using the formula:

$$\% Cytotoxicity = 100 [ T - ( {T}_{Eab} - E ) ] / [ T - {T}_{dead} ] where :$$

$$T = target cells$$

$${T}_{Eab} = treated target and effector cells$$

$$E = effector cells$$

$${T}_{dead} = target cells lysed with 0.1 \% Triton X - 100$$

Data graphs were plotted, and curve fit and EC50 values were calculated as described above. Error bars were calculated to represent standard error values.

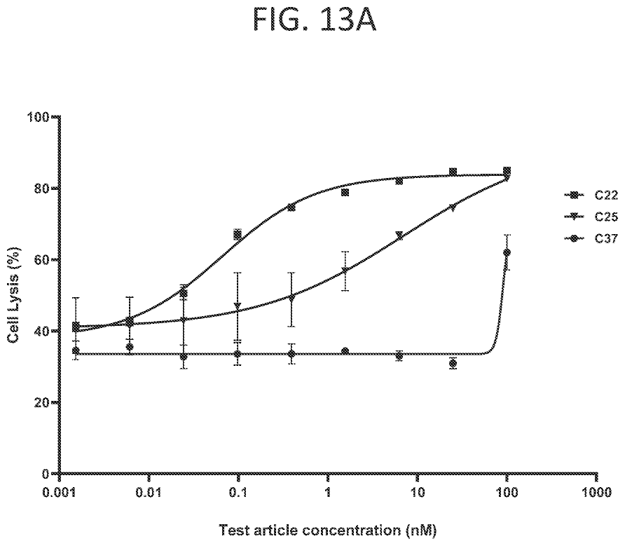

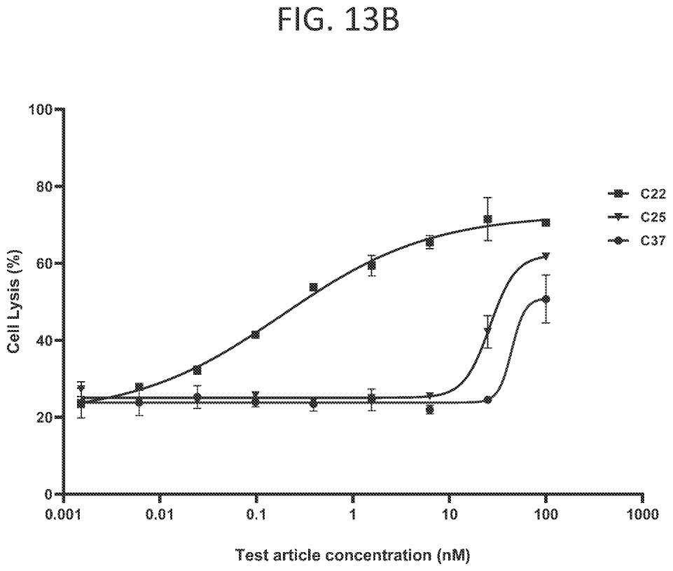

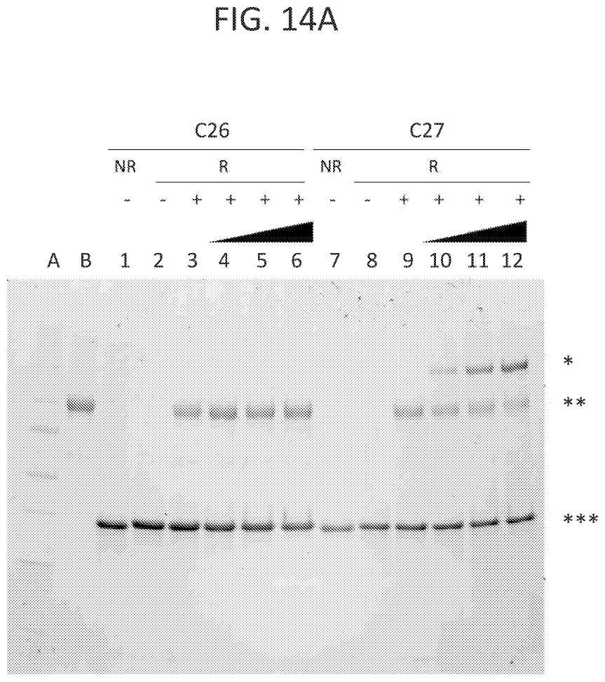

The FSY-containing NK cell engager C22 was demonstrated to be 106 times more potent than the non-covalent C25 in inducing specific target cell killing when present throughout the duration of the experiment (FIG. 13A) and 140 times more potent when incubated for 5 hr with target cells and washed out prior to adding effector cells (FIG. 13B). This increased potency can be ascribed to the ability of the C22 construct to covalently bind the target cell-expressed PSMA and is consistent with the superior binding to the target cells mediated by covalency. The results are tabulated in Table 8 and Table 9.

TABLE 8
EC₅₀ and Eₘₐₓ parameters of various constructs tested
for efficacy against specific target cell killing

PC3pip, no washout, 20 h incubation

| Compound | C22 | C25 | C37 |
|---|---|---|---|
| EC₅₀ [nM] | 0.066 | 7.021 | 90.47 |
| EC₅₀ CI [nM] | 0.044 to 0.092 | ND | ND |
| R squared | 0.9893 | 0.8222 | 0.8976 |
| Eₘₐₓ | 83.88 | 92.67 | ND |

TABLE 9
EC₅₀ and Eₘₐₓ parameters of various constructs
tested for efficacy against specific target cell
killing with intermediate washout at 5 hours

PC3pip, 5 h washout, 20 h incubation

| Compound | C22 | C25 | C37 |
|---|---|---|---|
| EC50 [nM] | 0.188 | 26.23 | 44.09 |
| EC50 CI [nM] | 0.076 to 0.363 | 23.03 to 42.35 | ND |
| R² | 0.9802 | 0.9777 | 0.8702 |
| Emax (%) | 72.77 | 62.18 | 50.90 |

### Example 9: Crosslinking of PSMA and T-Cell Bispecific Constructs

Additional bispecific conjugates were generated having a PSMA-targeted sdAb, with C35 (SEQ ID NO: 50) and C36 without (SEQ ID NO: 51) an incorporated FSY and T cell engager sdAb C38 or C39 targeted to CD3-epsilon (CD3ε) (SEQ ID NO: 53 or SEQ ID NO: 54) or C40 targeted the T-cell receptor alpha/beta (TCRαβ) (SEQ ID NO: 55), joined by a 4× (G4S) linker (L3; SEQ ID NO: 32), substantially as described in Example 5. The intact masses of all bispecific conjugates were confirmed by liquid chromatography-mass spectrometric (LC-MS) analysis, and the preparations were determined to be 92.8-99.7% pure by high-performance liquid chromatography-size exclusion chromatography (HPLC-SEC). The conjugates are summarized in Table 10 below:

TABLE 10
Constructs and corresponding SEQ ID NOs

| Compound # | SEQ ID NO |
|---|---|
| C26 | 41 |
| C27 | 42 |
| C28 | 43 |
| C29 | 44 |
| C30 | 45 |
| C31 | 46 |
| C32 | 47 |
| C33 | 48 |
| C34 | 49 |

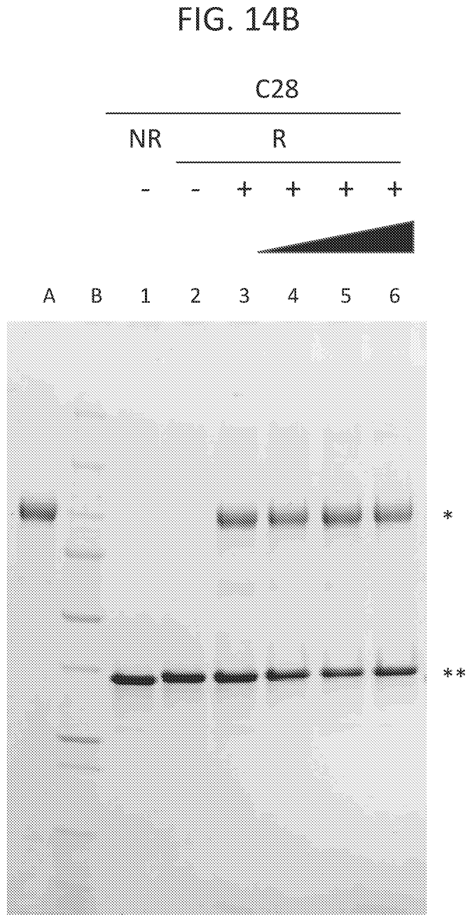

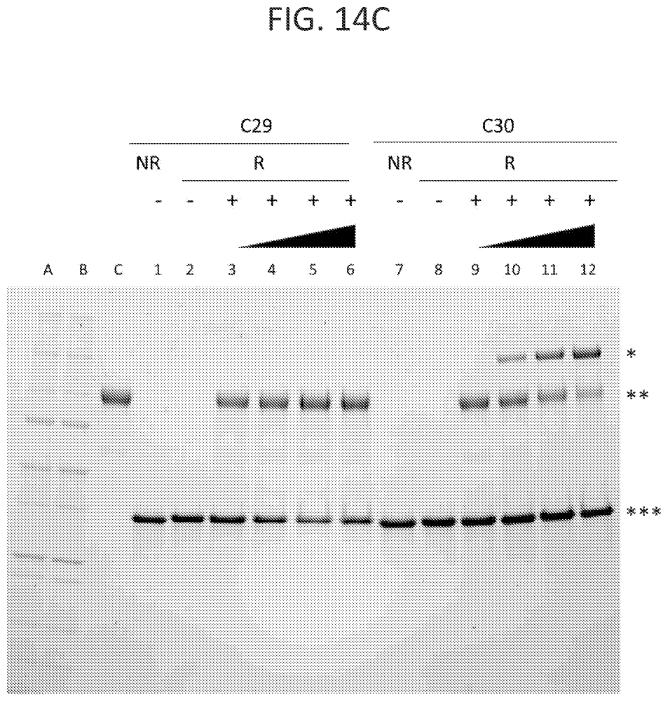

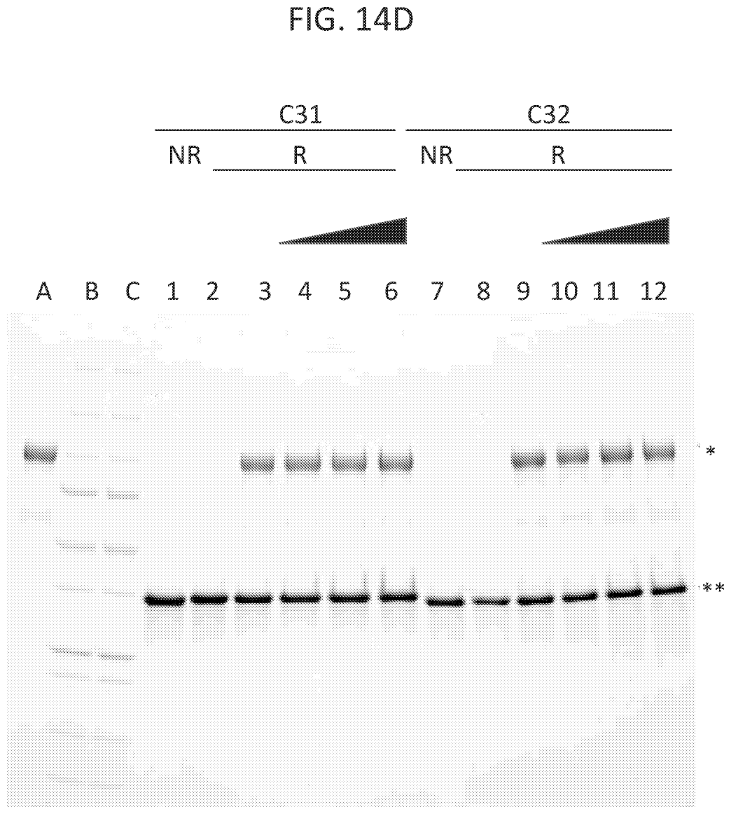

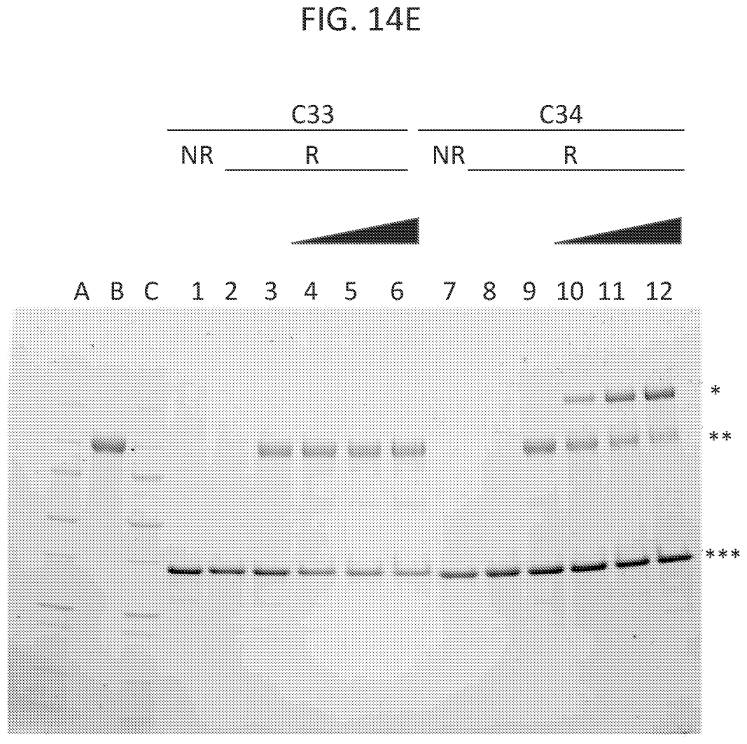

The purified bispecific sdAb constructs were incubated with PSMA (+PSMA) (Sino Biological; Cat #15877-H07H) at a molar ratio of ˜5:1 in 1×PBS (pH 7.4) for 0, 1, 5, or 10 minutes, or without PSMA (−PSMA; control), at 37° C. Following incubation, the reactions were quenched with SDS-Laemmli loading dye (1× final concentration with 100 mM DTT), heated to 95° C. for 5 min and analyzed by SDS-PAGE (4-20% Mini-PROTEAN® TGX™) with Coomassie blue staining. As shown in FIGS. 14A-14E, each bispecific variant was expressed and purified in sufficient quantities to assess crosslinking. All FSY-containing constructs demonstrated time-dependent crosslinking with PSMA. The ability to crosslink with PSMA was specific to FSY, as the bispecific variants containing a TYR substitution instead of FSY did not crosslink with PSMA.

Binding to immobilized recombinant human PSMA was determined by BLI substantially as described in Example 6. All constructs tested had highly similar binding sensorgrams (data not shown) and estimated affinities.

To assess the ability of C26 (with TYR) and C27 (with FSY) to crosslink PSMA, C26 and C27 were incubated with PSMA at varying concentrations, as described above. A sample of the incubated mixture was taken at 0, 1, 5, and 10 minutes and quenched with SDS-Laemmli loading dye, as described above. An SDS-PAGE was run of the collected samples and illustrated in FIG. 14A. Lanes A, B, and 1-12 are labeled as follows: A. Ladder; B. PSMA only; 1. Non-reduced C26 (no DTT) without PSMA; 2. Reduced (DTT) C26 without PSMA; 3. C26 with PSMA at 0 minutes; 4. C26 with PSMA after 1 minute; 5. C26 with PSMA after 5 minutes; 6. C26 with PSMA after 10 minutes; 7. Non-reduced C27 (no DTT) without PSMA; 7. Reduced (DTT) C27 without PSMA; 9. C27 with PSMA at 0 minutes; 10. C27 with PSMA after 1 minute; 11. C27 with PSMA after 5 minutes; 12. C27 with PSMA after 10 minutes. Different bands are marked with asterisks: * corresponds to the crosslinked test article with PSMA; ** corresponds to PSMA; *** corresponds to the test articles, C26 or C27.

To assess the ability of C28 (with TYR) to crosslink PSMA, C28 was incubated with PSMA at varying concentrations, as described above. A sample of the incubated mixture was taken at 0, 1, 5, and 10 minutes and quenched with SDS-Laemmli loading dye, as described above. An SDS-PAGE was run of the collected samples and illustrated in FIG. 14B. Lanes A, B, and 1-6 are labeled as follows: A. PSMA only; B. Ladder; 1. Non-reduced C28 (no DTT) without PSMA; 2. Reduced (DTT) C28 without PSMA; 3. C28 with PSMA at 0 minutes; 4. C28 with PSMA after 1 minute; 5. C28 with PSMA after 5 minutes; 6. C28 with PSMA after 10 minutes. Different bands are marked with asterisks: * corresponds to PSMA; ** corresponds to the test article, C28.

To assess the ability of C29 (with TYR) and C30 (with FSY) to crosslink PSMA, C29 and C30 were incubated with PSMA at varying concentrations, as described above. A sample of the incubated mixture was taken at 0, 1, 5, and 10 minutes and quenched with SDS-Laemmli loading dye, as described above. An SDS-PAGE was run of the collected samples and illustrated in FIG. 14C. Lanes A, B, C, and 1-12 are labeled as follows: A. and B. Ladder; C. PSMA only; 1. Non-reduced C29 (no DTT) without PSMA; 2. Reduced (DTT) C29 without PSMA; 3. C29 with PSMA at 0 minutes; 4. C29 with PSMA after 1 minute; 5. C29 with PSMA after 5 minutes; 6. C29 with PSMA after 10 minutes; 7. Non-reduced C30 (no DTT) without PSMA; 7. Reduced (DTT) C30 without PSMA; 9. C30 with PSMA at 0 minutes; 10. C30 with PSMA after 1 minute; 11. C30 with PSMA after 5 minutes; 12. C30 with PSMA after 10 minutes. Different bands are marked with asterisks: * corresponds to the crosslinked test article with PSMA; corresponds to PSMA; *** corresponds to the test articles, C29 or C30.

To assess the ability of PSMA-T-cell-engaging constructs to crosslink PSMA, C31 (with TYR) and C32 (with FSY) were incubated with PSMA at varying concentrations, as described above. A sample of the incubated mixture was taken at 0, 1, 5, and 10 minutes and quenched with SDS-Laemmli loading dye, as described above. An SDS-PAGE was run of the collected samples and illustrated in FIG. 14D. Lanes A, B, C, and 1-12 are labeled as follows: A. PSMA only; B. and C. Ladder; 1. Non-reduced C31 (no DTT) without PSMA; 2. Reduced (DTT) C31 without PSMA; 3. C31 with PSMA at 0 minutes; 4. C31 with PSMA after 1 minute; 5. C31 with PSMA after 5 minutes; 6. C31 with PSMA after 10 minutes; 7. Non-reduced C32 (no DTT) without PSMA; 7. Reduced (DTT) C32 without PSMA; 9. C32 with PSMA at 0 minutes; 10. C32 with PSMA after 1 minute; 11. C32 with PSMA after 5 minutes; 12. C32 with PSMA after 10 minutes. Different bands are marked with asterisks: * corresponds to PSMA; ** corresponds to the test articles, C31 or C32.

To assess the ability of PSMA-T-cell-engaging constructs to crosslink PSMA, C33 (with TYR) and C34 (with FSY) were incubated with PSMA at varying concentrations, as described above. A sample of the incubated mixture was taken at 0, 1, 5, and 10 minutes and quenched with SDS-Laemmli loading dye, as described above. An SDS-PAGE was run of the collected samples and illustrated in FIG. 14E. Lanes A, B, C, and 1-12 are labeled as follows: A. Ladder, B. PSMA; C. Ladder; 1. Non-reduced C33 (no DTT) without PSMA; 2. Reduced (DTT) C33 without PSMA; 3. C33 with PSMA at 0 minutes; 4. C33 with PSMA after 1 minute; 5. C33 with PSMA after 5 minutes; 6. C33 with PSMA after 10 minutes; 7. Non-reduced C34 (no DTT) without PSMA; 7. Reduced (DTT) C34 without PSMA; 9. C34 with PSMA at 0 minutes; 10. C34 with PSMA after 1 minute; 11. C34 with PSMA after 5 minutes; 12. C34 with PSMA after 10 minutes. Different bands are marked with asterisks: * corresponds to the crosslinked test article with PSMA; ** corresponds to PSMA; *** corresponds to the test articles, C33 or C34.

### Example 10: ADCC Activity Induced by T-Cell Bispecific Constructs

The impact of covalent linkage to target cells on the ability for bivalent constructs to promote T-cell activity in the presence of target cells expressing different levels of PSMA was tested. PC3 pip cells was determined substantially as described in Example 8, except incubating cells and constructs for 6 hr. instead of 4 hr. and the constructs described in Example 10 were used. An IgG1 isotype control antibody (BioXCell) was used as a negative control, and an anti-PSMA-Anti-CD3 IgG format bispecific antibody (BPS Bioscience Cat. No. 101242-2) was used as a positive control.

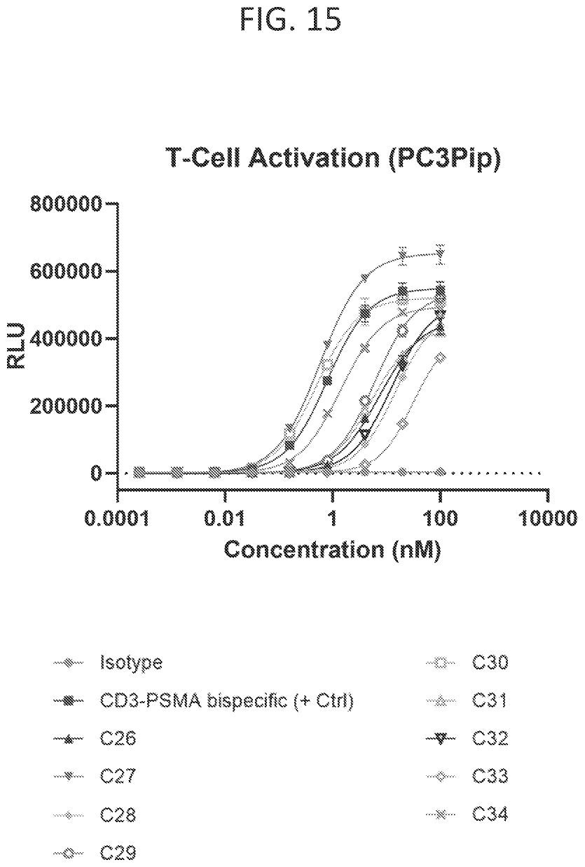

As shown in FIG. 15, the 6 hr. exposure to the tested T-cell bispecific constructs in the presence of PSMA-expressing PC3pip target cells resulted in a robust and dose-dependent induction of luminescence signal. The isotype control antibody (filled circle) did not elicit any response. In general, the FSY-modified T-cell bispecific constructs (C27, CD3-PSMA bispecific, C30, and C34) showed greater potency compared to their TYR-containing counterparts. In some cases, the FSY-equipped compounds were characterized not only by higher potency, but also higher maximal signal, compared to the non-covalent binders (FIGS. 16A-C). As shown in FIG. 16A, FSY-modified compound C27 demonstrated 12.7 times higher potency than the TYR-containing analog C26 (EC50 of 0.59 nM and 7.4 nM, respectively). Covalent binder C27 was also 1.4 times more potent and 1.2 times more efficacious than the commercial CD3-PSMA bispecific antibody used as a positive control. Covalent binder C30 showed 11.8 times higher potency over its non-covalent counterpart C29 (EC50 of 0.51 nM vs. 5.98 nM; FIG. 16B). C30 was also similarly efficacious and 1.6 times more potent than the commercial CD3-PSMA bispecific antibody (EC50 of 0.8 nM). FSY-modified compound C34 21.9 times more potent than its TYR-containing analog C33 (EC50 of 1.42 nM vs. 31.09 nM; FIG. 16C).

Three TYR-equipped T-cell bispecific constructs C28, C31, and C32 all effectively activated reporter cells with EC50 values of 14.76 nM, 5.33 nM, and 1.42 nM, respectively.

When tested in the presence of PSMA-negative PC-3 target cells, no substantial levels of T-cell activation by the T-cell bispecific constructs were observed, thus demonstrating the dependency of the activation on PSMA expression (FIG. 17). By comparison, the commercial CD3-PSMA bispecific antibody exhibited some T-cell activation at about 50 to about 100 nM CD3-PSMA bispecific antibody, whereas the FSY-containing constructs did not appear to stimulate T-cell activation.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Disclosure of the present application is further illustrated in the following list of embodiments, which are given for illustration purposes only and are not intended to limit the disclosure in any way:

Embodiment 1: A conjugate comprising a first targeting domain configured to bind a first target on a first cell, and a second targeting domain configured to bind a second target on a second cell, wherein the first targeting domain comprises at least one first unnatural amino acid (UAA) whereby the first targeting domain is capable of covalently binding to a first target at the site of the UAA to the first target.

Embodiment 2: The conjugate of embodiment 1, wherein the second cell is an immune cell.

Embodiment 3: The conjugate of embodiment 1 or embodiment 2, wherein the first cell is a tumor cell.

Embodiment 4: The conjugate of any one of embodiments 1-3, wherein the first targeting domain comprises an antibody or an antigen binding fragment thereof.

Embodiment 5: The conjugate of embodiment 4, wherein the first targeting domain comprises a single domain antibody (sdAb).

Embodiment 6: The conjugate of any one of embodiments 1-5, wherein the first UAA is comprised within or within proximity of a region of the first targeting domain that interfaces with the first target.

Embodiment 7: The conjugate of any one of embodiments 1-6, wherein the second targeting domain comprises an antibody or an antigen binding fragment thereof.

Embodiment 8: The conjugate of embodiment 7, wherein the second targeting domain comprises a single domain antibody (sdAb).

Embodiment 9: The conjugate of any one of embodiments 1-8, wherein the first targeting domain and the second targeting domain are joined as a fusion protein.

Embodiment 10: The conjugate of any one of embodiments 1-8, wherein the first targeting domain and the second targeting domain are joined by chemical conjugation.

Embodiment 11: The conjugate of embodiment 9 or embodiment 10, wherein the first targeting domain and the second domain are joined by a linker.

Embodiment 12: The conjugate of embodiment 11, wherein the linker is a polypeptide linker.

Embodiment 13: The conjugate of any one of embodiments 1-2, wherein the first target is a first cell surface molecule.

Embodiment 14: The conjugate of any one of embodiments 1-3, wherein the second target is a second cell surface molecule.

Embodiment 15: The conjugate of any one of embodiments 1-14, wherein the at least one first UAA comprises a fluoro sulfate moiety.

Embodiment 16: The conjugate of any one of embodiments 1-15, wherein the at least one UAA comprises an aryl-fluoro sulfate moiety.

Embodiment 17: The conjugate of embodiment 16, wherein the at least one first UAA comprises Formula I:

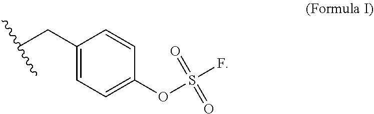 [SMILES: *Cc1ccc(OS(=O)(=O)F)cc1]

Embodiment 18: The conjugate of embodiment 16, wherein the at least one first UAA has the structure:

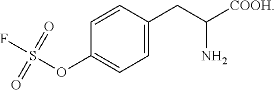 [SMILES: NC(Cc1ccc(OS(=O)(=O)F)cc1)C(=O)O]

Embodiment 19: The conjugate of embodiment 16, wherein the at least one first UAA has the structure:

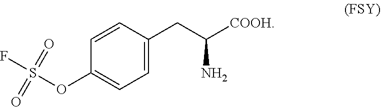 [SMILES: N[C@@H](Cc1ccc(OS(=O)(=O)F)cc1)C(=O)O]

Embodiment 20: The conjugate of embodiment 16, wherein the at least one first UAA comprises Formula II:

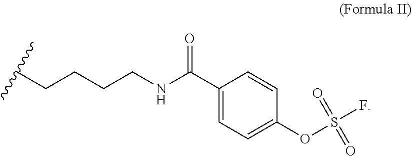 [SMILES: *CCCCNC(=O)c1ccc(OS(=O)(=O)F)cc1]

Embodiment 21: The conjugate of embodiment 16, wherein the at least one first UAA has the structure:

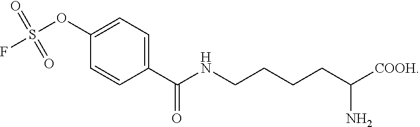 [SMILES: NC(CCCCNC(=O)c1ccc(OS(=O)(=O)F)cc1)C(=O)O]

Embodiment 22: The conjugate of embodiment 16, wherein the at least one first UAA has the structure:

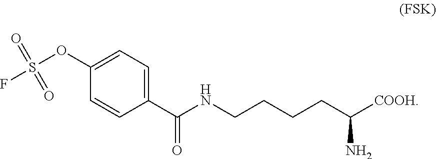 [SMILES: N[C@@H](CCCCNC(=O)c1ccc(OS(=O)(=O)F)cc1)C(=O)O]

Embodiment 23: The conjugate of embodiment 16, wherein the at least one first UAA comprises Formula III:

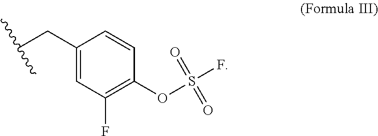 [SMILES: *Cc1ccc(OS(=O)(=O)F)c(F)c1]

Embodiment 24: The conjugate of embodiment 16, wherein the at least one first UAA has the structure:

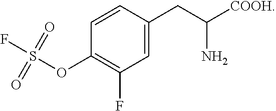 [SMILES: NC(Cc1ccc(OS(=O)(=O)F)c(F)c1)C(=O)O]

Embodiment 25: The conjugate of embodiment 16, wherein the at least one first UAA has the structure:

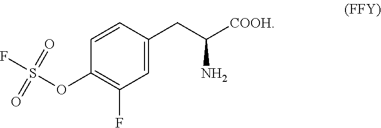 [SMILES: N[C@@H](Cc1ccc(OS(=O)(=O)F)c(F)c1)C(=O)O]

Embodiment 26: The conjugate of any one of embodiments 1-15, wherein the at least one first UAA has a structure of Formula (IA):

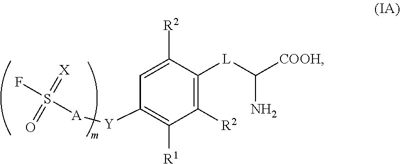

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, mis 1; when Y is —N═, mis 2.

Embodiment 27: The conjugate of embodiment 26, wherein the at least one first UAA has a structure of Formula (IA-a):

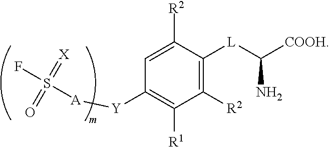

Embodiment 28: The conjugate of embodiment 26, wherein the at least one first UAA has a structure of Formula (IA-b):

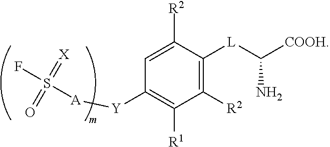

Embodiment 29: The conjugate of embodiment 26, wherein the at least one first UAA has a structure of Formula (IB):

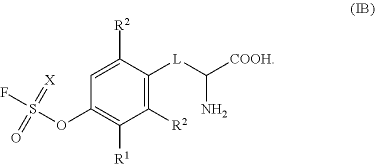

Embodiment 30: The conjugate of embodiment 26, wherein the at least one first UAA has a structure of Formula (IC):

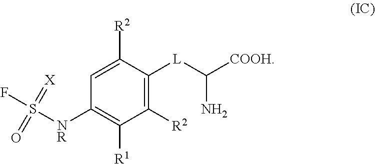

Embodiment 31: The conjugate of embodiment 26, wherein the at least one first UAA has a structure of Formula (ID):

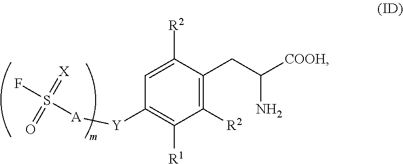

wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

Embodiment 32: The conjugate of embodiment 26, wherein the at least one first UAA has a structure of Formula (IE):

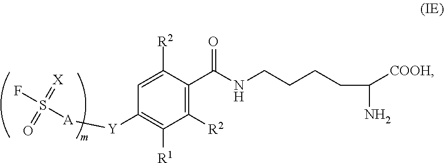

wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

Embodiment 33: The conjugate of embodiment 26, wherein the at least one first UAA has a structure of Formula (IIA):

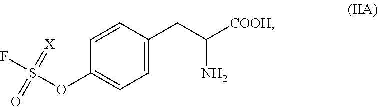

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Embodiment 34: The conjugate of embodiment 26, wherein the at least one first UAA has a structure of Formula (IIB):

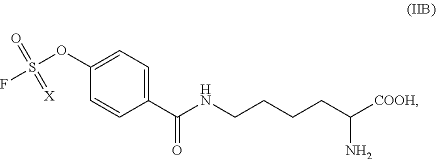

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Embodiment 35: The conjugate of any one of embodiments 1-15, wherein the at least one first UAA has a structure of

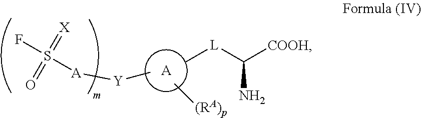

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, mis 2.

Embodiment 36: The conjugate of any one of embodiments 13-35, wherein the first cell surface molecule is selected from the group consisting of PSMA, EGFR, HER2, HER3, CD3, CD16, NKD46, PD-L1, EphA4, Fibronectin ED-B, CD45, EpCAM, CCR4, CD25, VEGF, VEGFR2, endo180, LIV-1, PTK7, CDH6, CSP-1, CD71, PRLR, SEZ6, DLL1, NOTCH3 rec, NaPi2b, DLK1, Muc16, LRP5, and LRP6.

Embodiment 37: The conjugate of any one of embodiments 14-36, wherein the second cell surface molecule is a cell surface molecule present on an immune cell.

Embodiment 38: The conjugate of embodiment 37, wherein the immune cell is a T-cell.

Embodiment 39: The conjugate of embodiment 37, wherein the immune cell is a NK-cell.

Embodiment 40: The conjugate of embodiment 37, wherein the immune cell is a gamma-delta T cell.

Embodiment 41: The conjugate of embodiment 37, wherein the second cell surface molecule is CD3, NKD44, NKD46, NKD30, NKG2D, γδ TCR, Vδ1, Vγ9Vδ2, or CD16.

Embodiment 42: The conjugate of any one of embodiments 1-41, wherein the second domain comprises a second UAA, whereby the second domain is capable of covalently binding to the second target at the site of the second UAA.

Embodiment 43: The conjugate of any embodiment 42, wherein the second UAA is different from the at least one UAA comprised in the first targeting domain.

Embodiment 44: The conjugate of embodiment 42, wherein the second UAA is the same as the at least one UAA comprised in the first targeting domain.

Embodiment 45: The conjugate of any one of embodiments 42-44, wherein the second UAA comprises a fluoro sulfate moiety.

Embodiment 46: The conjugate of any one of embodiments 42-44, wherein the second UAA comprises an aryl-fluoro sulfate moiety.

Embodiment 47: The conjugate of embodiment 46, wherein the second UAA comprises Formula I:

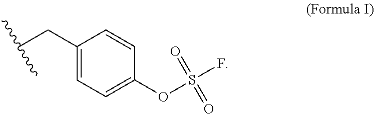 [SMILES: *Cc1ccc(OS(=O)(=O)F)cc1]

Embodiment 48: The conjugate of embodiment 46, wherein the second UAA has the structure:

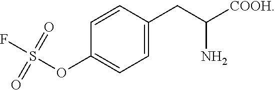 [SMILES: NC(Cc1ccc(OS(=O)(=O)F)cc1)C(=O)O]

Embodiment 49: The conjugate of embodiment 46, wherein the second UAA has the structure:

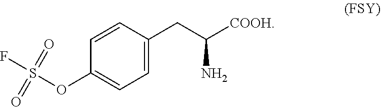 [SMILES: N[C@@H](Cc1ccc(OS(=O)(=O)F)cc1)C(=O)O]

Embodiment 50: The conjugate of embodiment 46, wherein the second UAA comprises Formula II:

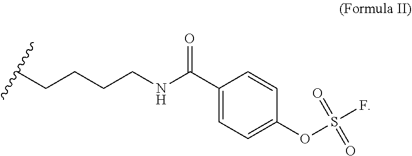 [SMILES: *CCCCNC(=O)c1ccc(OS(=O)(=O)F)cc1]

Embodiment 51: The conjugate of embodiment 46, wherein the second UAA has the structure:

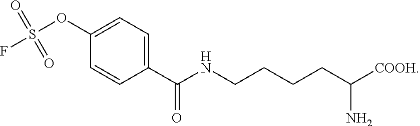 [SMILES: NC(CCCCNC(=O)c1ccc(OS(=O)(=O)F)cc1)C(=O)O]

Embodiment 52: The conjugate of embodiment 46, wherein the second UAA has the structure:

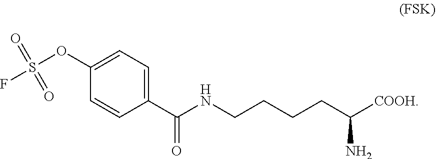 [SMILES: N[C@@H](CCCCNC(=O)c1ccc(OS(=O)(=O)F)cc1)C(=O)O]

Embodiment 53: The conjugate of embodiment 46, wherein the second UAA comprises Formula III:

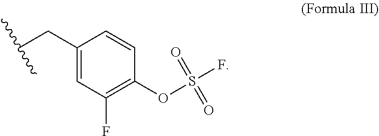 [SMILES: *Cc1ccc(OS(=O)(=O)F)c(F)c1]

Embodiment 54: The conjugate of embodiment 46, wherein the second UAA has the structure:

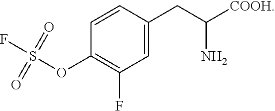 [SMILES: NC(Cc1ccc(OS(=O)(=O)F)c(F)c1)C(=O)O]

Embodiment 55: The conjugate of embodiment 46, wherein the second UAA has the structure:

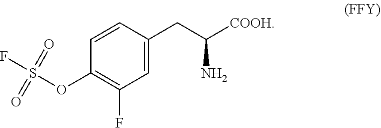 [SMILES: N[C@@H](Cc1ccc(OS(=O)(=O)F)c(F)c1)C(=O)O]

Embodiment 56: The conjugate of any one of embodiments 42-44, wherein the second UAA has a structure of Formula (IA):

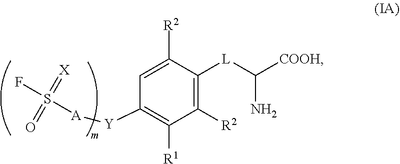

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer of 1 to 6; and
wherein when Y is —O— or —NR—, mis 1; when Y is —N═, mis 2.

Embodiment 57: The conjugate of embodiment 56, wherein the second UAA has a structure of Formula (IA-a):

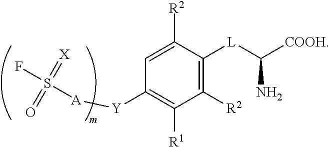

Embodiment 58: The conjugate of embodiment 56, wherein the second UAA has a structure of Formula (IA-b):

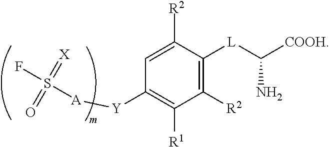

Embodiment 59: The conjugate of embodiment 56, wherein the second UAA has a structure of Formula (IB):

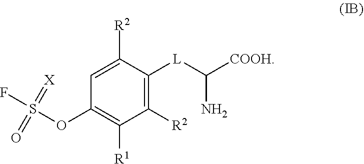

Embodiment 60: The conjugate of embodiment 56, wherein the second UAA has a structure of Formula (IC):

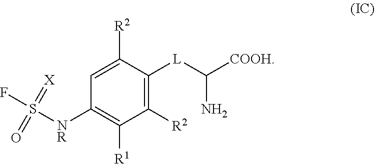

Embodiment 61: The conjugate of embodiment 56, wherein the second UAA has a structure of Formula (ID):

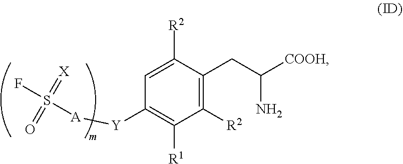

wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

Embodiment 62: The conjugate of embodiment 56, wherein the second UAA has a structure of Formula (IE):

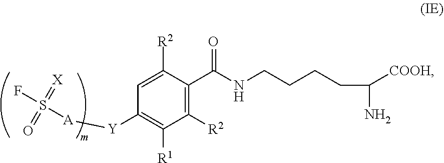

wherein:
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

Embodiment 63: The conjugate of embodiment 56, wherein the second UAA has a structure of Formula (IIA):

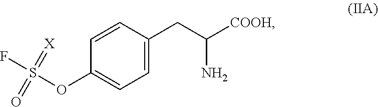

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Embodiment 64: The conjugate of embodiment 56, wherein the second UAA has a structure of Formula (IIB):

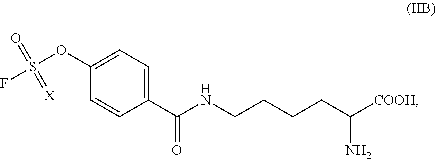

wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

Embodiment 65: The conjugate of any one of embodiments 42-44, wherein the second UAA has a structure of

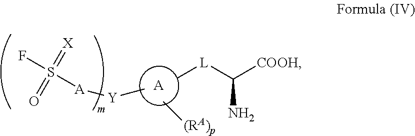

wherein,
each X is independently O or NR′;
Y is a bond, —O—, —NR—, or —N═;
A is a bond or —(CH₂)ₙ—; m is 1 or 2; n is an integer of 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
Ring A is a 5- to 6-membered aryl or heteroaryl;
each R^{A} is independently —OH, —OR^{X}, halogen, NHR^{X}, N(R^{X})₂, or optionally substituted alkyl; p is 0, 1, 2, 3 or 4; each R^{X} is optionally substituted alkyl;
L is —(CH₂)ₚ— or —C(O)NH—(CH₂)ₚ—; p is an integer of 1 to 6; and
wherein when Y is a bond, —O— or —NR—, m is 1; when Y is —N═, mis 2.

Embodiment 66: The conjugate of any one of embodiments 13-35, wherein the first cell surface molecule is selected from the group consisting of PSMA, EGFR, HER2, HER3, CD3, CD16, NKD46, PD-L1, EphA4, Fibronectin ED-B, CD45, EpCAM, CCR4, CD25, VEGF, VEGFR2, endo180, LIV-1, PTK7, CDH6, CSP-1, CD71, PRLR, SEZ6, DLL1, NOTCH3 rec, NaPi2b, DLK1, Muc16, LRP5, and LRP6.

Embodiment 67: The conjugate of any one of embodiments 14-36, wherein the second cell surface molecule is a cell surface molecule present on an immune cell.

Embodiment 68: The conjugate of embodiment 65, wherein the immune cell comprises a T-cell or a NK-cell.

Embodiment 69: The conjugate of embodiment 65, wherein the immune cell is a gamma-delta T cell.

Embodiment 70: The conjugate of embodiment 65, wherein the second cell surface molecule is CD3, CD16, TCRαβ, NKD44, NKD46, NKD30, NKG2D, γδ TCR, Vδ1, or Vγ9Vδ2.

Embodiment 71: The conjugate of any one of embodiments 1-65, wherein the first targeting domain comprises any one of SEQ ID NOs: 1-4, 16-18, 20, and 29.

Embodiment 72: The conjugate of any one of embodiments 1-65, wherein the first targeting domain comprises a sequence having at least 70% sequence identity to any one of SEQ ID NOs: 1-4, 16-18, 20, 29, 50, or 51.

Embodiment 73: The conjugate of any one of embodiments 1-66, wherein the second targeting domain comprises SEQ ID NO: 22, 25, or 52-55.

Embodiment 74: The conjugate of any one of embodiments 1-66, wherein the second targeting domain comprises a sequence having at least 70% sequence identity to SEQ ID NO: 22, 25, or 52-55.

Embodiment 75: The conjugate of any one of embodiments 1-65, wherein the conjugate comprises any one of SEQ ID NOs: 19, 23, 24, 26-28, 30, or 35-49.

Embodiment 76: The conjugate of any one of embodiments 1-65, wherein the conjugate comprises a sequence having at least 70% sequence identity to any one of SEQ ID NOs: 19, 23, 24, 26-28, 30, or 35-49.

Embodiment 77: The conjugate of any one of embodiments 1-65, wherein the conjugate comprises a sequence having at least 70% sequence identity to any one of SEQ ID NOs: 1-3, 16, 18, 22, 25, or 50-55.

Embodiment 78: The conjugate of any one of embodiments 1-65, wherein the conjugate comprises a sequence having at least 70% sequence identity to any one of SEQ ID NOs: 19, 21, 23, 24, 26-28, 30, or 35-49.

Embodiment 79: The conjugate of any one of embodiments 1-65, wherein the first targeting domain comprises SEQ ID NO: 1.

Embodiment 80: The conjugate of embodiment 75, wherein the UAA is present at an amino acid position selected from the group consisting of 26, 28, 29, 30, 99, 102, 103, 105, 108, 110, 111, 112, 113, 114, and 115 relative to SEQ ID NO: 1.

Embodiment 81: The conjugate of any one of embodiments 1-65, wherein first targeting domain comprises SEQ ID NO: 2.

Embodiment 82: The conjugate of embodiment 77, wherein the UAA is present at an amino acid position selected from the group consisting of 50, 52, 53, 54, 56, 58, and 100 relative to SEQ ID NO:2.

Embodiment 83: The conjugate of any one of embodiments 1-65, wherein the first targeting domain comprises SEQ ID NO: 3.

Embodiment 84: The engineered sdAb of embodiment 79, wherein the UAA is present at an amino acid position selected from the group consisting of 58, 62, 101, 103, and 107 relative to SEQ ID NO: 3.

Embodiment 85: The conjugate of any one of embodiments 1-65, wherein the first targeting domain comprises SEQ ID NO: 16.

Embodiment 86: The conjugate of embodiment 75, wherein the first targeting domain comprising SEQ ID NO: 16 further comprises an unnatural amino acid at position 109 relative to SEQ ID NO: 16.

Embodiment 87: The conjugate of any one of embodiments 1-65, wherein the first targeting domain comprises SEQ ID NO: 18.

Embodiment 88: The conjugate of any one of embodiments 1-83, wherein the second targeting domain comprises SEQ ID NO: 22.

Embodiment 89: The conjugate of any one of embodiments 1-83, wherein the second targeting domain comprises SEQ ID NO: 25.

Embodiment 90: A method comprising administering the conjugate of any one of embodiments 1-85, wherein the conjugate covalently binds the first target on the surface of a first cell.

Embodiment 91: A method comprising administering the conjugate of any one of embodiments 1-85, wherein the conjugate binds the second target on the surface of a second cell.

Embodiment 92: A method comprising administering the conjugate of any one of embodiments 1-85, wherein the conjugate covalently binds the first target on the surface of a first cell and the conjugate binds the second target on a second cell.

Embodiment 93: The method of any one of embodiments 86-89, wherein the second cell is a tumor cell.

Embodiment 94: The method of embodiment 90, wherein the conjugate kills or inhibits the growth of the tumor cell.

Embodiment 95: The method of embodiment 90 or 91, wherein the first target is selected from the group consisting of PSMA, EGFR, HER2, HER3, PD-L1, EphA4, Fibronectin ED-B, CD45, EpCAM, CCR4, CD25, VEGF, VEGFR2, endo180, LIV-1, PTK7, CDH6, CSP-1, CD71, PRLR, SEZ6, DLL1, NOTCH3 rec, NaPi2b, SIRPa, DLK1, Muc 16, LRP5, and LRP6.

Embodiment 96: The method of embodiment 86-92, wherein the immune cell is a T-cell.

Embodiment 97: The method of embodiment 86-93, wherein the immune cell is a gamma-delta T cell.

Embodiment 98: The method of embodiment 86-92, wherein the immune cell is a NK-cell or a NKT-cell.

Embodiment 99: The method of embodiment 86-95, wherein the second cell surface molecule is CD3, CD16, CD16a, CD3ε, TCRαβ, NKD44, NKD46, NKD30, NKG2D, γδTCR, Vδ1, or Vγ9Vδ2.

Embodiment 100: A method of treating a proliferative disease or disorder in a subject in need thereof, comprising administering to the subject a therapeutically effective amount of the conjugate of any one of embodiments 1-85.

Embodiment 101: The method of embodiment 97, wherein the proliferative disease or condition is a cancer.

Embodiment 102: The method of embodiment 98, wherein the cancer is a solid tumor cancer selected from the group consisting of bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, and prostate cancer.

Embodiment 103: A method of manufacturing the conjugate of any one of embodiments 1-85 comprising synthesizing the first targeting domain comprising at least one un natural amino acid in vivo.

Embodiment 104: The method of embodiment 100, further comprising synthesizing the second domain in vivo.

Embodiment 105: The method of embodiment 100 or 101, wherein synthesizing comprises use of an orthogonal tRNA synthetase/suppressor tRNA pair.

Embodiment 106: The method of embodiment 100, wherein synthesizing comprises the orthogonal tRNA synthetase/suppressor tRNA pair is derived from pyrrolysine tRNA synthetase/tRNA^{Pyl}.

What is claimed:

1. A conjugate comprising:
a first targeting domain configured to bind a first target on a first cell, and
a second targeting domain configured to bind a second target on a second cell,
wherein the first targeting domain comprises at least one first unnatural amino acid (UAA) whereby the first targeting domain is capable of covalently binding to a first target at the site of the UAA to the first target.

2. The conjugate of claim 1, wherein the second cell is an immune cell.

3. The conjugate of claim 1 or claim 2, wherein the first cell is a tumor cell.

4. The conjugate of any one of claims 1-3, wherein the first targeting domain comprises an antibody or an antigen binding fragment thereof.

5. The conjugate of claim 4, wherein the first targeting domain comprises a single domain antibody (sdAb).

6. The conjugate of any one of claims 1-5, wherein the first UAA is comprised within or within proximity of a region of the first targeting domain that interfaces with the first target.

7. The conjugate of any one of claims 1-6, wherein the second targeting domain comprises an antibody or an antigen binding fragment thereof.

8. The conjugate of claim 7, wherein the second targeting domain comprises a single domain antibody (sdAb).

9. The conjugate of any one of claims 1-8, wherein the first targeting domain and the second targeting domain are joined by chemical conjugation.

10. The conjugate of claim 9, wherein the first targeting domain and the second domain are joined by a linker.

11. The conjugate of claim 10, wherein the linker is a polypeptide linker.

12. The conjugate of any one of claim 1 or 2, wherein the first target comprises a first cell surface molecule.

13. The conjugate of any one of claims 1-3, wherein the second target is a second cell surface molecule.

14. The conjugate of any one of claims 1-13, wherein the at least one UAA comprises an aryl-fluoro sulfate moiety.

15. The conjugate of claim 14, wherein the at least one first UAA comprises Formula I:

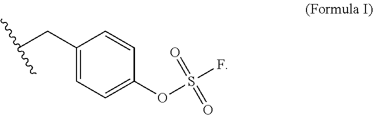 [SMILES: *Cc1ccc(OS(=O)(=O)F)cc1]

16. The conjugate of claim 14, wherein the at least one first UAA has the structure:

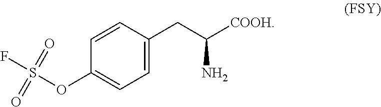 [SMILES: N[C@@H](Cc1ccc(OS(=O)(=O)F)cc1)C(=O)O]

17. The conjugate of claim 14, wherein the at least one first UAA comprises Formula II:

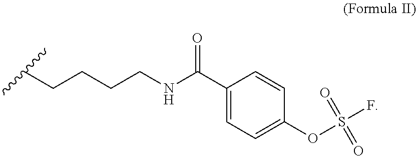 [SMILES: *CCCCNC(=O)c1ccc(OS(=O)(=O)F)cc1]

18. The conjugate of claim 14, wherein the at least one first UAA has the structure:

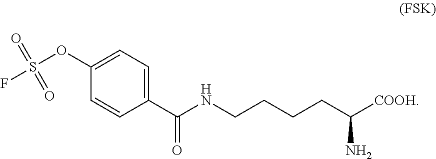 [SMILES: N[C@@H](CCCCNC(=O)c1ccc(OS(=O)(=O)F)cc1)C(=O)O]

19. The conjugate of claim 14, wherein the at least one first UAA comprises Formula III:

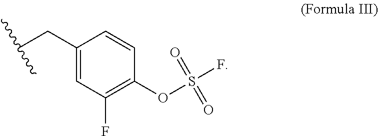 [SMILES: *Cc1ccc(OS(=O)(=O)F)c(F)c1]

20. The conjugate of claim 14, wherein the at least one first UAA has the structure:

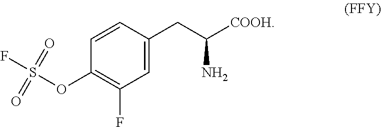 [SMILES: N[C@@H](Cc1ccc(OS(=O)(=O)F)c(F)c1)C(=O)O]

21. The conjugate of any one of claims 1-14, wherein the at least one first UAA has a structure of Formula (IA): 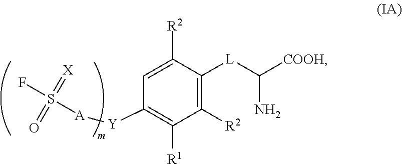
wherein,
each X is independently O or NR′;
Y is a bond, —O—, —NR—, or —N═;
A is a bond or —(CH₂)ₙ—; m is 1 or 2; n is an integer from 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl;
L is —(CH₂)ₚ— or —C(O)NH—(CH₂)ₚ—; p is an integer from 1 to 6; and
wherein when Y is —O— or —NR—, mis 1; when Y is —N═, mis 2.

22. The conjugate of claim 21, wherein the at least one first UAA has a structure of Formula (IA-a):

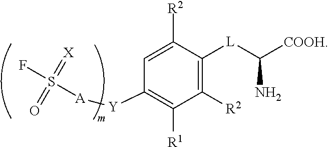

23. The conjugate of claim 21, wherein the at least one first UAA has a structure of Formula (IB):

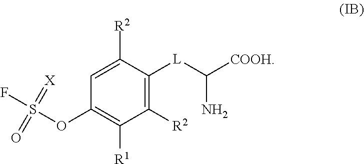

24. The conjugate of claim 21, wherein the at least one first UAA has a structure of Formula (ID): 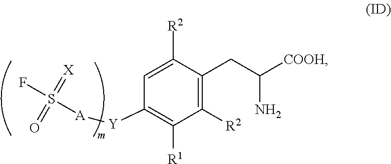
wherein:
each X is independently O or NR′;
Y is a bond, —O—, —NR—, or —N═;
A is a bond or —(CH₂)ₙ—; m is 1 or 2; n is an integer from 1 to 4;
each R and R′, when present, is independently hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl;
R¹ is hydrogen, fluoro, or iodo; R² is hydrogen or methyl; and
wherein when Y is a bond, —O— or —NR—, m is 1; when Y is —N═, mis 2.

25. The conjugate of claim 21, wherein the at least one first UAA has a structure of Formula (IIA): 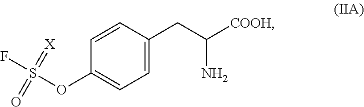
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

26. The conjugate of claim 21, wherein the at least one first UAA has a structure of Formula (IIB): 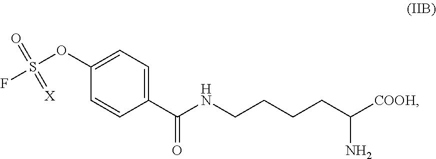
wherein:
X is independently O or NR′; and
R′, when present, is hydrogen, substituted or unsubstituted alkyl, substituted or unsubstituted heteroalkyl, substituted or unsubstituted aryl, substituted or unsubstituted heteroaryl, or substituted or unsubstituted heterocycloalkyl.

27. The conjugate of any one of claims 12-26, wherein the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT.

28. The conjugate of any one of claims 13-27, wherein the second cell surface molecule is a cell surface molecule present on an immune cell.

29. The conjugate of claim 28, wherein the immune cell comprises a T-cell or a NK-cell.

30. The conjugate of claim 28, wherein the second cell surface molecule is CD3, CD16, TCRαβ, NKD44, NKD46, NKD30, NKG2D, γδTCR, Vδ1, or Vγ9Vδ2.

31. The conjugate of any one of claims 1-30, wherein the first targeting domain comprises any one of SEQ ID NOs: 1-4, 16-18, 20, 29, 50, or 51.

32. The conjugate of any one of claims 1-31, wherein the first targeting domain comprises a sequence having at least 70% sequence identity to any one of SEQ ID NOs: 1-4, 16-18, 20, 29, 50, or 51.

33. The conjugate of any one of claims 1-31, wherein the second targeting domain comprises a sequence having at least 70% sequence identity to SEQ ID NO: 22, 25, or 52-55.

34. The conjugate of any one of claims 1-31, wherein the conjugate comprises a sequence having at least 70% sequence identity to any one of SEQ ID NOs: 19, 21, 23, 24, 26-28, 30, 35-49, 57, or 64.

35. The conjugate of any one of claims 1-34, wherein the first targeting domain comprises SEQ ID NO: 16 or 51.

36. The conjugate of claim 35, wherein the first targeting domain comprising SEQ ID NO: 16 further comprises an unnatural amino acid at position 109 relative to SEQ ID NO: 16.

37. The conjugate of claim 35, wherein the first targeting domain comprising SEQ ID NO: 51 further comprises an unnatural amino acid at position 10¹ relative to SEQ ID NO: 51.

38. A method comprising administering the conjugate of any one of claims 1-37, wherein the conjugate covalently binds the first target on the surface of a first cell.

39. A method comprising administering the conjugate of any one of claims 1-37, wherein the conjugate binds the second target on the surface of a second cell.

40. A method comprising administering the conjugate of any one of claims 1-37, wherein the conjugate covalently binds the first target on the surface of a first cell and the conjugate binds the second target on a second cell.

41. The method of any one of claims 38-40, wherein the first cell is a tumor cell.

42. The method of claim 41, wherein the first target comprises 5T4, B7-H3, B7-4, BCMA, CAIX, CD123, CD19, CD20, CD22, CD30, CD33, CD79b, CEA, DLL3, EGFR, EGFRviii, FAP, FcRH5, GPR20, GUCY2C (GCC), HER2, HER3, KAAG1, LIV-1, MICA, MSLN, Muc1, BCMA, C4.4a, CA6, CAIX, CanAg, CAXII, CCR2, CCR4, CCR7, CD103, CD123, CD13, CD138, CD142, ENPP3, EpCAM, EphA2, EphA3, EphA4, ETBR, FAP, FcRH5, FGFR2, FGFR3, FLT3, FolRa, GD2, GD3, MRC2, MSLN, MT1-MMP, MTX7, Muc1, Muc16, NaPi2b, NECTIN4, UCHT1, VCAM-1, VEGF, VEGFR2, VpreB, VPREB1, or xCT.

43. The method of claim 40, wherein the second cell is an immune cell, wherein the immune cell is a T-cell or a NK cell.

44. The method of claim 40, wherein the second cell surface molecule is CD3, CD16, TCRαβ, NKD44, NKD46, NKD30, NKG2D, γδTCR, Vδ1, or Vγ9Vδ2.

45. A method of treating a proliferative disease or disorder in a subject in need thereof, comprising administering to the subject a therapeutically effective amount of the conjugate of any one of claims 1-37.

46. The method of claim 45, wherein the proliferative disease or condition is a cancer.

47. A method of manufacturing the conjugate of any one of claims 1-37 comprising synthesizing the first targeting domain comprising at least one unnatural amino acid in vivo.

48. The method of claim 47, further comprising synthesizing the second domain in vivo.

49. The method of claim 47 or 48, wherein synthesizing comprises use of an orthogonal tRNA synthetase/suppressor tRNA pair.

50. The method of claim 49, wherein synthesizing comprises the orthogonal tRNA synthetase/suppressor tRNA pair is derived from pyrrolysine tRNA synthetase/tRNA^{Pyl}.
